(19) United States
(12) Patent Application Publication
(10) Pub. No.: US 2019/0054135 A1
(43) Pub. Date: Feb. 21, 2019

(54) PLANT OR MICROORGANISM-DERIVED CAROTENOID-OXYGEN COPOLYMER COMPOSITIONS, METHODS OF IDENTIFYING, QUANTIFYING AND PRODUCING SAME AND USES THEREOF

(71) Applicant: Avivagen Inc., Ottawa (CA)

(72) Inventors: Graham BURTON, Ottawa (CA); Janusz DAROSZEWSKI, Ottawa (CA); Trevor J. MOGG, Ottawa (CA); Grigory B. NIKIFOROV, Kanata (CA); James G. NICKERSON, Charlottetown (CA); Cameron L. GROOME, Mississauga (CA)

(21) Appl. No.: 16/079,190

(22) PCT Filed: Feb. 27, 2017
(86) PCT No.: PCT/CA2017/050254

§ 371 Date: Aug. 23, 2018

Related U.S. Application Data
(60) Provisional application No. 62/299,737, filed on Feb. 25, 2016.

(51) Int. Cl. A61K 36/81; A61K 36/899; A23L 33/105

(52) U.S. Cl. CPC A61K 36/81; A61K 36/899; A23L 33/105; A61K 2236/30; A61K 2236/53; A23V 2300/40; A23V 2200/324; A23V 2250/211; A23V 2300/14; A23V 2300/21; A23V 2002/00

(57) ABSTRACT

The present invention relates to carotenoid-oxygen copolymers, compositions, methods of identifying and quantifying carotenoid-oxygen copolymers in food and related sources, and methods of producing compositions comprising same. In one aspect the method of identifying and quantifying carotenoid-oxygen copolymers comprises an analysis of a low molecular weight marker compound in said sources. In another aspect the present invention provides a method of preparing compositions comprising said carotenoid-oxygen copolymers and/or enhancing levels of said copolymers in food sources in a sufficient and practically useful concentration to have beneficial effects in animals and humans, including beneficial immunological and health effects.

### FIELD OF THE INVENTION

The invention relates to carotenoid-oxygen copolymer compositions, methods of identifying and quantifying carotenoid-oxygen copolymers from natural sources, such as natural food sources, such as plant sources or microorganism sources, and methods of producing said compositions. The invention also contemplates compositions comprising effective amounts of carotenoid-oxygen copolymers for various uses, such as to maintain and enhance the overall health of animals and humans or to enhance the immune response or immunity of an animal or human

### BACKGROUND OF THE INVENTION

Various health benefits are ascribed to dietary carotenoids.¹⁻³ The several provitamin A carotenoids, including α- and β-carotenes and β-cryptoxanthin, provide benefits linked to their vitamin A activities.⁴ However, less easily explained are other, non-vitamin A benefits of both provitamin A carotenoids and of other carotenoids that cannot be converted into vitamin A.⁵⁻⁷

Carotenoids are yellow, orange, and red pigments synthesized by plants. There are over 600 known carotenoids that are made up of two classes called carotenes, which are purely hydrocarbons, and xanthophylls, which are carotenes substituted with one or a few oxygen atoms. β-Carotene, and lycopene are examples of common carotenes, whereas lutein, zeaxanthin, and canthaxanthin are common examples of xanthophylls. The most common carotenoids in North American diets are α-carotene, β-carotene, β-cryptoxanthin, lutein, zeaxanthin, and lycopene.

All carotenoids are formed from 8 isoprene units and each carotenoid molecule contains 40 carbon atoms. Structurally, carotenoids take the form of a polyene hydrocarbon chain, which is sometimes terminated at one or both ends by a ring. Carotenoids that contain unsubstituted β-ionone rings (including β-carotene, α-carotene, β-cryptoxanthin and γ-carotene) have vitamin A activity (meaning that they can be converted to retinal). By contrast, lutein, zeaxanthin, capsanthin, canthaxanthin and lycopene have no vitamin A activity.

Traditionally, non-vitamin A activities have been ascribed to actions of the carotenoid itself,⁸⁻¹⁰ often as an antioxidant. However, recent research casts doubt upon an antioxidant role, at least with regard to inhibiting carcinogenesis, and points to the operation of other mechanisms.¹¹⁻¹²

Although it has been long known that addition of oxygen is inherently favored in spontaneous oxidation of highly unsaturated compounds,¹⁵ the predominant involvement and the significance of oxidative polymerization of carotenoids had surprisingly escaped notice prior to the inventors' reports^{13, 14} (also see U.S. Pat. No. 5,475,006; U.S. Pat. No. 7,132,458; U.S. Pat. No. 8,211,461; US 2011-0217244; US 2013-0131183; and US 2013-0156816). Furthermore, the studies with a fully-oxidized β-carotene composition (termed OxBC, the active ingredient in Avivagen Inc.'s OxC-Beta™ branded products) obtained by spontaneous reaction of β-carotene with oxygen in a solvent as well with the analogously formed fully oxidized lycopene, have revealed that the polymeric fraction is responsible for immunological activity,¹⁴ which includes an ability to prime and enhance innate immune function¹⁴ as well as to limit inflammatory processes.¹⁶ Carotenoids other than β-carotene and lycopene have not been previously studied as a source of immunologically active polymers of this type.

Further, given the ubiquity of carotenoids, including and especially β-carotene, and their known susceptibility to loss during processing of food,^{17, 18} it is unclear whether and to what extent oxidation and, in particular, copolymerization occur naturally in foods and may account for this loss.

There is a need to determine whether carotenoid oxidation products themselves have beneficial properties, for instance non-vitamin A health benefits, and/or whether it is the parent carotenoid and its antioxidant action that has such benefits. Further there is a need to develop products, such as animal feed, animal and human supplements and foods that can enhance the health of animals and humans. Further, there is a need to identify sources of oxidized carotenoid products, to develop oxidized carotenoid products from natural sources (such as food sources, plants, or microorganisms). Further, there is a need to find economical sources of such oxidized carotenoid products and methods for producing same.

### SUMMARY OF THE INVENTION

In some embodiments of the invention, the inventors have surprisingly identified natural sources, such as food plant sources (e.g. plants or parts thereof, fruits, and vegetables), and microorganisms, that are a good source of carotenoid-oxygen copolymers. Further, the inventors in some embodiments, have surprisingly been able to produce carotenoid-oxygen copolymer compositions and products from natural carotenoid sources.

In some embodiments, said natural sources can be used for non-vitamin A carotenoid associated health benefits. In some embodiments the plant sources and microorganism comprise high level of carotenoids that during processing under aerobic conditions can result in a product with carotenoid-oxygen copolymers. In some other embodiments, the non-processed plant source or microorganism may also have carotenoid-oxygen copolymers and can be used directly or processed in a manner to not only isolate the carotenoid-oxygen copolymer component (or isolate the component comprising the carotenoid-oxygen copolymer), but in some embodiments to also enhance carotenoid-oxygen copolymer content of the resulting product. Thus in some embodiments, the methods of the present invention result in products comprising carotenoid oxygen copolymers with beneficial effects, without starting from an isolated or purified carotenoid, but rather by taking a starting product rich in carotenoids such as a natural source, and oxidizing the carotenoids in situ. In some embodiments, the starting product is already rich in carotenoid-oxygen copolymers.

In some embodiments, the inventors have developed new carotenoid-oxygen copolymer comprising products from natural sources and methods of producing same. In some other embodiments, the methods of the present invention enable the production of products in a consistent manner that have a desired amount of carotenoid-oxygen copolymer. Said products have advantages for the uses noted herein, such as to enhance animal and human health. The ability to produce products consistently also has advantages from both a regulatory and consumer product point of view. As such, the present invention, in some embodiments provides a product comprising consistent levels of carotenoid-oxygen copolymers.

In some other embodiments, the inventors have developed a method for enhancing levels/concentration of carotenoid-oxygen copolymers in said natural sources and resulting compositions and products. In some other aspects of the invention the method comprises using plants or microorganisms genetically modified to increase levels of carotenoids to enhance the potential for carotenoid-oxygen copolymer production. In some other aspects, the invention provides a method for enhancing the resulting concentration of carotenoid-oxygen copolymer in the processed natural source product, by processing the natural source under oxidative polymerization conditions and recovering the copolymer comprising fraction(s) through one or more cycles of polar solvent extractions and non-polar solvent precipitations.

Further, unlike prior art compositions of OxBC, in some other embodiments, the inventors have been able to isolate and/or develop products that comprise carotenoid-oxygen copolymers and not norisoprenoid breakdown products. In some embodiments, the active ingredients of compositions and products of the invention are carotenoid-oxygen copolymers. In some other embodiments, said compositions and products are free from norisoprenoid breakdown products.

In some other embodiments, the products of the invention, in addition to carotenoid-oxygen copolymers may comprise carotenoids and non-fully oxidized carotenoids. In another embodiment, the product may comprise other oxidized non-carotenoid products, said composition depending on the natural source used. In some other embodiments, the product is a powder.

In certain aspects, the invention provides a method of identifying a source of carotenoid-oxygen copolymers comprising:

(a) selecting a source containing carotenoids, wherein in one embodiment, said source is a food plant source or a microorganism source including but not limited to bacteria, yeast fungi, and algae. In one example the sources are genetically modified to enhance levels of carotenoids, such as golden rice and M37W-Ph3 corn and genetically modified microorganisms, such as yeast, or as described by G. Guiliano in “Plant carotenoids: genomics meets multi-gene engineering” Current Opinion in Plant Biology 2014, 19:111-117⁵⁴;

(b) processing the source under oxidative polymerization conditions, such as exposure to oxygen, increasing surface area of exposure to oxygen, increasing the partial pressure of oxygen (ppO₂) and/or temperature or in a manner that enhances the level of carotenoid-oxygen copolymer present in the source; and

(c) quantifying the amount of carotenoid-oxygen copolymer by directly isolating or identifying same from said processed source and/or by isolating or identifying an indicator of same from said processed source, to determine whether it is a source of carotenoid-oxygen copolymer. In some embodiments, the sources have a starting amount of carotenoid, which may provide upon oxidation the same amount of carotenoid-oxygen copolymer of 1-1000 μg/g wet weight or 10-10,000 μg/g dry weight. In some embodiments sources resulting in a desired carotenoid-oxygen copolymer level, such as 10-10,000 μg/g dry weight are selected.

In yet some other embodiments, the plant source is selected from the group consisting of: carrots, tomato, alfalfa, spirulina, rosehip, sweet pepper, chili pepper, paprika, sweet potato, kale, spinach, seaweed, wheatgrass, marigold⁴⁴⁻⁴⁸, moringa oleifera⁴⁹⁻⁵² and red palm oil. In another embodiment, the sources are plant products that are powders, e.g. carrot powder, tomato powder, spirulina powder, rosehip powder, paprika powder, seaweed powder, and wheatgrass powder.

In some embodiments, the microorganism source is selected from the group consisting of: bacteria, yeast, fungi, and algae, such as spirulina⁴⁴ and forms of same genetically modified to increase carotenoid levels to enhance carotenoid-oxygen copolymer yields. In some further embodiments, the microorganisms are selected from the group of the following species: Algae: Spirulina, Dunaliella, Haematococcus, Murielopsis. Fungi: Blakeslea trispora. Yeasts: Xanthophyllomyces dendrorhous, Rhodotorula glutinis. Bacteria: Sphingomonas.

In some embodiments, the carotenoid has an unsubstituted β-ionone ring structure and the indicator is geronic acid. In another embodiment, the carotenoid with the unsubstituted β-ionone ring structure is selected from one or more of: β-cryptoxanthin; α-carotene; γ-carotene; and β-carotene, or in another embodiment, β-carotene.

In some other embodiments the carotenoid is selected from those that do not form vitamin A, or do not have vitamin A activity, such as the carotenoid is selected from lycopene, lutein, zeaxanthin, capsanthin and canthaxanthin.

In some embodiments the indicator for the presence of carotenoid-oxygen copolymers are as follows: (i) geronic acid for the carotenoid-oxygen copolymers of β-cryptoxanthin; α-carotene; β-carotene, and γ-carotene; (ii) geranic acid for the carotenoid-oxygen copolymers of lycopene and γ-carotene; (iii) 4-hydoxygeronic acid and/or its lactone for the carotenoid-oxygen copolymers of lutein, zeaxanthin, and capsanthin; and (iv) 2,2-dimethylglutaric acid and its anhydride for the carotenoid-oxygen copolymer of canthaxanthin. In some embodiments the present invention provides a method of determining the presence of the aforementioned carotenoid-oxygen copolymers by detecting (through isolation, labeling, methyl esterification or other means) their respective indicators. In some embodiments, one can use said indicators to quantify the presence of said carotenoid-oxygen copolymers by quantifying the amount of said indicators and correlating said amount to an amount of the carotenoid-oxygen copolymer.

In some embodiments, the oxidative polymerization conditions are selected from exposure to air or oxygen and one or more of drying, powdering, increasing exposure to heat, light, increasing the partial pressure of oxygen (ppO₂) and/or temperature, or other factors that promote oxidation. In another embodiment, the isolating of carotenoid-oxygen copolymer comprises one or more solvent extraction/precipitation cycles. In certain embodiments, the solvent for extraction is a polar organic solvent, such as ethyl acetate or butyl acetate. In other aspects of the invention, the precipitation is conducted using a non-polar solvent such as hexane, pentane or heptane, or in some embodiments, hexane.

In some other embodiments, the method of identifying is selected from one or more of: elemental analysis, GC-MS, GPC and FTIR.

In some other aspects, the invention provides a method of preparing a product containing carotenoid-oxygen copolymers, said method comprising:

(a) obtaining a natural source containing carotenoids or in some embodiments, rich in carotenoids, such as a microorganism or a food plant source, a yeast, a fungus, algae or a bacteria. In some embodiments, the natural sources are selected from the plant and microorganisms previously noted; and

(b) processing said source under oxidative polymerization conditions. In some embodiments, said conditions are selected from exposure to air or oxygen and one or more of drying, powdering, increasing exposure to heat, light, increasing the partial pressure of oxygen (ppO₂) and/or temperature, or other factors that promote oxidation and/or conditions that enhance the level of carotenoid-oxygen copolymers present.

In yet some other embodiments, the invention provides a method for isolating a carotenoid-oxygen copolymer product by subjecting the product obtained from (b) above, to one or more cycles of polar organic solvent, e.g. ethyl acetate extractions/non-polar solvent precipitation, e.g. hexane, pentane or heptane or, in one embodiment, hexane, and recovering the carotenoid-oxygen copolymer containing fraction from same. In one embodiment, the solvents used in the process would be selected from those that are generally recognized as safe (GRAS). The extraction/precipitation cycles result in a carotenoid-oxygen copolymer product that does not contain carotenoid breakdown products that may have been formed during the oxidation process.

In some embodiments, the invention comprises a composition comprising the carotenoid-oxygen copolymer isolated in accordance with the methods of the present invention and optionally suitable excipients. In some other embodiments, the invention provides an animal feed or supplement for an animal feed comprising carotenoid-oxygen copolymer or product or composition containing same developed pursuant to the present invention. In some embodiments, the product is naturally sourced (for instance from foods, such as plants, such as fruits or vegetables or from microorganisms, such as algae, fungi (such as yeast), or bacteria). In yet some other embodiments the invention provides a nutraceutical or supplement or food comprising a carotenoid-oxygen copolymer or product or composition containing same developed pursuant to the methods of the present invention for human or animal use.

In some other embodiments, the invention provides a method for enhancing carotenoid-oxygen copolymer in a source, such as food source or supplement (such as a plant derived food or supplement) comprising the steps of processing said food source or supplement under oxidizing conditions to enhance the formation and/or isolation of carotenoid-oxygen copolymer and/or copolymer comprising fractions.

In some other embodiments, the invention provides a use of carotenoid-oxygen copolymers and compositions comprising same to enhance animal and human health, and/or immunity in an animal, such as selected from one or more of: enhancing innate immunity, limiting or reducing inflammation, enhancing the functioning of the immune system, enhancing the ability of an animal to resist disease, recover or overcome disease or maintain a healthy state. In some embodiments, the source of carotenoid is a food or plant or other source. In certain embodiments an enriched carrot powder or tomato powder comprising said oxidized carotenoid products could be used directly in animal feed for livestock (for instance, 2-4 kg of carrot powder to match 2 ppm of synthetically derived, e.g., OxBC in 1 tonne of feed) or the ‘pure’ isolated oxidized carotenoid polymer product derived from said natural sources could be used in dog or cat chew supplements.

Further, in some other aspects the present invention provides products that prime and enhance innate immune function and limit chronic inflammation. Further, the oxidized carotenoids of the present invention that are food-derived copolymers are formed from a blend of carotenoids within the environment of the food itself rather than in an organic solvent. The resultant mixed copolymers could then be used in the form of the powder or could be isolated in more or less pure form by solvent extraction/precipitation for use including food supplements and cosmetics.

Additional aspects and advantages of the present invention will be apparent in view of the description which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 illustrates in a model of the oxidative polymerization of a carotenoid that the spontaneous reaction of β-carotene with molecular oxygen in an organic solvent generates predominantly β-carotene-oxygen copolymers together with the mostly familiar short chain norisoprenoid compounds. Full oxidation of β-carotene is highly reproducible, consuming almost 8 molar equivalents of molecular oxygen with an accompanying increase in weight of ca. 30% in the final product, OxBC. Other examples of carotenoids, including lycopene, lutein and canthaxanthin, behave in a very similar manner indicating that oxidative polymerization is a general phenomenon common to the carotenoid family, which is comprised of approximately 600 members. The model reaction is used as a basis to determine if carotenoids present in plant-derived foods and related substances undergo a similar reaction to give similar products.

FIG. 2A illustrates that oxidative polymerization of provitamin A (PVA) carotenoids generates geronic acid (GA) as a minor product by a double oxidative cleavage of the carbon skeleton. β-carotene with two conjugated β-ionone rings can yield two GA per molecule. GA is the most abundant of the norisoprenoid products generated from β-carotene, α-carotene, γ-carotene and β-cryptoxanthin, with one β-ionone ring each can yield just one GA per molecule.

FIG. 2B illustrates that oxidative polymerization of the non-provitamin A carotenoid lycopene generates geranic acid as a minor product by an oxidative cleavage of the carbon skeleton. In some embodiments, lycopene may yield two geranic acids per molecule while γ-carotene can only generate one. It should be noted that geranic acid can also be an indicator for γ-carotene.

FIG. 2C illustrates that oxidative polymerization of the non-provitamin A carotenoid lutein generates 4-hydoxygeronic acid and/or its lactone. This is also the indicator for the carotenoid-oxygen copolymers of zeaxanthin, and capsanthin.

FIG. 2D illustrates that oxidative polymerization of the non-provitamin A canthaxantin generates 2,2-dimethylglutaric acid and its anhydride.

FIG. 2E depicts the chemical structures of lycopene, γ-carotene, lutein, zeaxanthin, capsanthin and canthaxanthin.

FIG. 3 is a typical calibration curve of the GC-MS intensity ratio, I/I₆, of GA to GA-d₆ methyl esters plotted vs. known ratios of quantities of the two compounds, m/m₆.

FIG. 4 is a GC-MS chromatogram of a geronic acid analysis of a carrot juice sample recorded in SIM mode. Signals are of methyl esters of GA-d₆ (A, m/z=160) and GA (B, m/z=154).

FIG. 5 is a GC-MS chromatogram of a geronic acid analysis of a raw tomato sample recorded in SIM mode. Signals of methyl esters of GA-d₆ (A, m/z=160) and GA (B, m/z=154) are indicated.

FIG. 6 shows GC-MS chromatograms of geronic acid analyses of carrot juice (top) and raw tomato (bottom) samples recorded in scan mode. Retention times for methyl esters of GA-d₆ (A) and GA (B) are 7.43 and 7.46 min, respectively.

FIG. 7 shows FTIR spectra of polymer fractions isolated by successive solvent precipitations of extracts of carrot and tomato powder. In order starting from top: carrot powder #1 compared to fully oxidized β-carotene (OxBC) and tomato powder compared to fully oxidized lycopene (OxLyc).

FIG. 8 shows UV-Vis spectra in methanol solvent of the precipitated fraction obtained from extracted carrot powder #1 (dotted line) compared to the OxBC polymer (solid line).

FIG. 9 shows a GPC of the 3× precipitated fraction obtained from extracted carrot powder #1 (dotted line) compared to that of the OxBC polymer (solid line). UV absorbance was monitored at 220-400 nm. The amount injected was 200 μg for both samples. The median MW for the OxBC polymer at 7.7 min is approximately 700-800 Da. (Burton et al.¹³).

FIG. 10 shows GC-MS chromatograms of OxBC polymer (bottom) and the precipitated fraction obtained from extracted carrot powder #2 (top) following thermal decomposition in the GC injector port at 250° C. Compounds identified with a greater than 50% match with the GC-MS library, unless noted otherwise, are 1: β-cyclocitral; 2: β-homocyclocitral (2-(2,6,6-trimethylcyclohex-1-enyl) acetaldehyde); 3: 4,8-dimethylnona-1,7-dien-4-ol (38-47% match); 4: 5,6-epoxy-β-ionone; 5: dihydroactinidiolide; 6: 4-oxo-β-ionone. Peak 7 in the upper trace is identified as α-ionone (40% match).

FIG. 11 shows GPC chromatograms illustrating the polymeric nature of hexane-precipitated solids isolated from ethyl acetate extracts of (A) carrot powder #2, (B) tomato powder, (C) tomato pomace, (D) rosehip powder, (E) sun-cured alfalfa, (F) dulse seaweed powder, (G) wheatgrass powder, and (H) paprika.

FIG. 12 shows GPCs of polymer fractions isolated by hexane precipitation from ethyl acetate solutions of fully oxidized (A) lycopene (OxLyc), (B) lutein (OxLut) and (C) canthaxanthin (OxCan).

FIG. 13 shows FTIR spectra of hexane-precipitated polymeric solids isolated from ethyl acetate extracts of (in order starting from top): carrot powder #2, tomato pomace, rosehip powder, sun-cured alfalfa, wheatgrass powder, dulse seaweed powder, and paprika.

FIG. 14 shows FTIR spectra of fully oxidized canthaxanthin (OxCan) and lutein (OxLut).

FIG. 15 A shows the reaction scheme for esterification of geranic acid with Me₃OBF₄ to give methyl geranate (compound A); 15B shows the proposed synthesis of deuterium-labeled geranic acid; 15C is a GC chromatogram of tomato powder extract and OxLyc low MW fraction, esterified with Me₃OBF₄, where compound A has been identified by its mass spectrum. The difference in retention times are the result of minor differences in analytical run conditions.

FIG. 16 is a graph illustrating the formation of geronic acid with concomitant loss of β-carotene in dehydrated carrot upon standing in air and exposed to light. Measurements at time 0 used freshly dehydrated carrot purée, and subsequent time points were measured with dried carrot powder, spread thinly on a tray and exposed to air and light.

FIG. 17 are grey scale photographs (visual comparison) of carrot purée, day-1 (A); dehydrated carrot purée, day 0 (B); carrot powder, day 0 (C); and carrot powder, day 21 (D). In colour they are various shades of orange with (A) and (B) being darker than (C) which is darker than (D).

FIG. 18 are grey scale photographs (visual comparison) of the effect of limiting air exposure of carrot powder samples: (A) sample was prepared by grinding dehydrated carrot chips in a coffee blade mill, then sealing in a jar for 4 weeks and 6 days (in colour it was orange); (B) sample was prepared by powdering dried carrot purée with a food processor blade, grinding it with a coffee burr mill then exposing it to air for 1 week and 6 days (in colour it was brown).

FIG. 19 shows the low molecular weight marker of autoxidation of lutein, zeaxanthin and capsanthin: 19A shows the formation of 4,5-didehydromethyl geronate (compound B) by reaction of its parent lactone with Me₃OBF₄; 19B shows one possible synthesis of a deuterium-labeled marker, the lactone of 4-hydroxygeronic acid-d₆; and 19C shows GC chromatograms of dulse powder extract and the low MW fraction of OxLut, esterified with Me₃OBF₄, where the retention time of compound B is noted at 7.32 min. Common mass spectral ions include m/z=184 (M+), 152, 125, 109, 83, 81, 69, 55, 43.

FIG. 20 shows the low molecular weight marker of autoxidation of canthaxanthin: 20 (A) illustrates the conversion of 2,2-dimethylglutaric acid to its anhydride and its dimethyl ester (compound C); 20 (B) shows one possible synthesis of a deuterium-labeled marker, 2,2-dimethylglutaric acid, from isobutyric acid-d₆ starting material.

### DETAILED DESCRIPTION OF THE INVENTION

### Definitions/Abbreviations

Abbreviations Used: BHT, butylated hydroxy toluene (2,6-di-tert-butyl-4-methylphenol); GA, geronic acid; OxBC, fully oxidized β-carotene; OxLyc, fully oxidized lycopene; OxLut, fully oxidized lutein; OxCan, fully oxidized canthaxanthin; OxPVA, oxidized provitamin A carotenoids; PVA, provitamin A carotenoids; SPE, solid phase extraction.

“Animal” is meant any animal including, without limitation, humans, dogs, cats, horses, sheep, swine, cattle, poultry, and fish.

An “amount sufficient” or “effective amount” is meant the amount of oxidatively transformed carotenoid or carotenoid-oxygen polymer, or a fractionated component thereof, required to improve health, for instance to enhance the functioning of the immune system including priming innate immune function and limiting inflammatory processes, enhance the ability to resist disease, recover or overcome disease or maintain a healthy state, increase joint mobility, increase the activity level, or improve the coat quality. The effective amount of a composition of the invention used to practice the methods of the invention varies depending upon the manner of administration, the type of animal, body weight, and general health of the animal. Ultimately, the attending physician or veterinarian will decide the appropriate amount and dosage regimen. Such amount is referred to as an “amount sufficient” or “effective amount”.

“Carotenoid” as used herein refers to naturally-occurring pigments of the terpenoid group that can be found in plants, algae, bacteria, and certain animals, such as birds and shellfish. Carotenoids include but are not limited to carotenes, which are hydrocarbons (i.e., without oxygen), and their oxygenated derivatives (i.e., xanthophylls). Examples of carotenoids include lycopene; α-carotene; γ-carotene; β-carotene; echinenone; isozeaxanthin; canthaxanthin; citranaxanthin; β-apo-8′-carotenic acid ethyl ester; hydroxy carotenoids, such as alloxanthin, apocarotenol, astacene, astaxanthin, capsanthin, capsorubin, carotenediols, carotenetriols, carotenols, cryptoxanthin, β-cryptoxanthin, decaprenoxanthin, epilutein, fucoxanthin, hydroxycarotenones, hydroxyechinenones, hydroxylycopene, lutein, lycoxanthin, neurosporine, phytoene, phytofluoene, rhodopin, spheroidene, torulene, violaxanthin, and zeaxanthin; and carboxylic carotenoids, such as apocarotenoic acid, β-apo-8′-carotenoic acid, azafrin, bixin, carboxylcarotenes, crocetin, diapocarotenoic acid, neurosporaxanthin, norbixin, and lycopenoic acid.

“Carotenoid-Oxygen Copolymer”, “Carotenoid Copolymer” and “Polymer” as used herein refers to a carotenoid, which is an unsaturated compound, that has been fully oxidized at its reactive double bonds by spontaneous reaction with molecular oxygen, resulting in co-polymers of the carotenoid with oxygen as the main product and does not include or is separated and isolated from any accompanying norisoprenoid by-products.

“Comprising”, as used herein is synonymous with “including” and “containing”, and are inclusive or open-ended and does not exclude additional, un-recited elements or method steps.

“Consisting of”, as used herein is closed-ended and, subject to the doctrine of equivalents, excludes any element, step, or ingredient not specified in the claim.

To “enhance the functioning of the immune system, enhance the ability to resist disease, recover or overcome disease or maintain a healthy state” can be assessed in many ways, including but not limited to assessing an animal's health after exposure to disease-causing antigens, viruses, bacteria, or various stressors, its ability to not contract a disease after exposure or to recover from a disease compared to control animals.

“Fully Oxidized Carotenoid”, as used herein, refers to a carotenoid, which is an unsaturated compound, that has been fully oxidized at its reactive double bonds by spontaneous reaction with molecular oxygen, resulting in a mixture of copolymers of the carotenoid with oxygen and norisoprenoid breakdown products.

“GA”, as used herein, refers to geronic acid.

“Natural” or “Natural Source”, as used herein refers to plant sources (including plants or parts thereof, wherein the parts thereof may include but is not limited to seeds, leaves, and stems, fruits or vegetables) or microorganisms. “Natural Product” or “Naturally Sourced Product” refers to products derived from processing natural sources.

“Provitamin A Carotenoids” refer to those carotenoids that are capable of being converted by oxidation into vitamin A, including but not limited to, namely α-, β- and γ-carotenes and β-cryptoxanthin.

“OxBC” is a fully oxidized carotenoid composition that is the synthetic product of spontaneous reaction with oxygen of pure β-carotene comprising about 85% by weight of β-carotene-oxygen copolymers and about 15% low molecular weight breakdown products called norisoprenoids. Other carotenoid oxygen copolymer compositions derived from pure carotenoids are similarly designated, such as OxLut for fully oxidized lutein, OxLyc for fully oxidized lycopene or OxCan for fully oxidized canthaxanthin.

“OxPVA” is a carotenoid-oxygen copolymer composition comprising one or more fully oxidized provitamin A carotenoids (“PVA”) which may comprise other residual products (i.e., the carotenoid-oxygen copolymer and other oxygenated by-products). In reference to the example, such as Example 2, it refers to estimated total provitamin A carotenoid-oxidation copolymers present. “OxCar” refers to a carotenoid-oxygen copolymer composition comprising one or more fully oxidized carotenoids, whether provitamin A or not which may comprise other residual products (i.e., the carotenoid-oxygen copolymer and other oxygenated by-products). In some embodiments OxPVA and OXCar may comprise norisoprenoids.

### DESCRIPTION

Although highly unsaturated compounds are long-known to preferentially polymerize during oxidation, the predominance and significance of polymerization in carotenoid oxidation surprisingly had escaped notice before the work of the present inventors. Importantly, β-carotene-oxygen copolymers exhibit immunological activity, including priming of innate immune function and limiting inflammatory processes. The inventors' discovery of food (such as plant sources) containing carotenoid-oxygen copolymers, as disclosed herein, with anticipated non-vitamin A immunological activities has important health implications, including for human and animal nutrition. For instance, in one example as described herein, the chemical nature of the compound isolated from carrot powder (originally rich in β-carotene) was confirmed by comparing elemental analysis, GPC, IR, GC-MS thermolysis and UV data with those from OxBC. Elemental analysis, IR and GPC data of compounds isolated in the same manner from other dried foods supported their oxygen-copolymer nature.

Finding significant levels of such copolymers indicates that mechanisms involving the oxidation products, as opposed to an antioxidant action, of the parent carotenoid are responsible for non-vitamin A health benefits. Rather than a potential diminishment of purported parent carotenoid activity by its oxidative loss, the inventors herein disclose that carotenoids transformed into polymeric compounds have previously unrecognized beneficial immunological potential. This assertion is supported by the health benefits the inventors observed in studies in livestock and companion animals using diets supplemented with low parts-per-million OxBC. Here, a successful search in foods (such as dried foods) for natural sourced counterparts of such copolymers that are responsible for the non-vitamin A benefits of carotenoids is disclosed. In one embodiment, the products comprising carotenoid-oxygen copolymer(s) are made from products rich in carotenoids in situ as opposed to isolated or synthetic carotenoids.

β-Carotene-oxygen copolymers occur in common fresh or dried foods, including carrots, tomatoes, sweet potatoes, paprika, rosehips, seaweeds, alfalfa and milk, at levels encompassing an approximately thousand-fold range, from low parts-per-billion in fresh foods to parts-per-million in dried foods. Copolymers isolated from some dried foods reach parts-per-thousand levels—comparable to the original carotenoid levels. In vivo biological activity of supplemental β-carotene-oxygen copolymers has been previously documented at parts-per-million levels, suggesting certain foods have such activity.

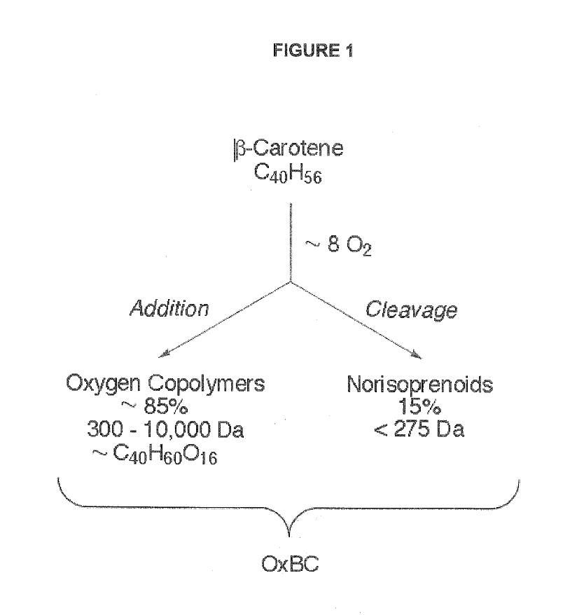

The inventors recently reported a novel finding that pure β-carotene and other carotenoids oxidize to form immunologically active, non-vitamin A products.^{13, 14} This finding implies non-vitamin A activity requires prior oxidative conversion of the carotenoid, just as for vitamin A activity. Importantly, the spontaneous reaction is characterized by addition of oxygen to form predominantly carotenoid-oxygen copolymer compounds, as well as minor amounts of the usual, mostly familiar, norisoprenoid breakdown products (FIG. 1).¹³ The inventors use this detailed understanding of the model oxidation of β-carotene in solution, which results in a highly reproducible product (OxBC) comprised of β-carotene-oxygen copolymers (ca. 85% w/w) and norisoprenoid compounds (ca. 15%) (FIG. 1)¹³ to develop a method of identifying natural sources of carotenoid-oxygen copolymers.

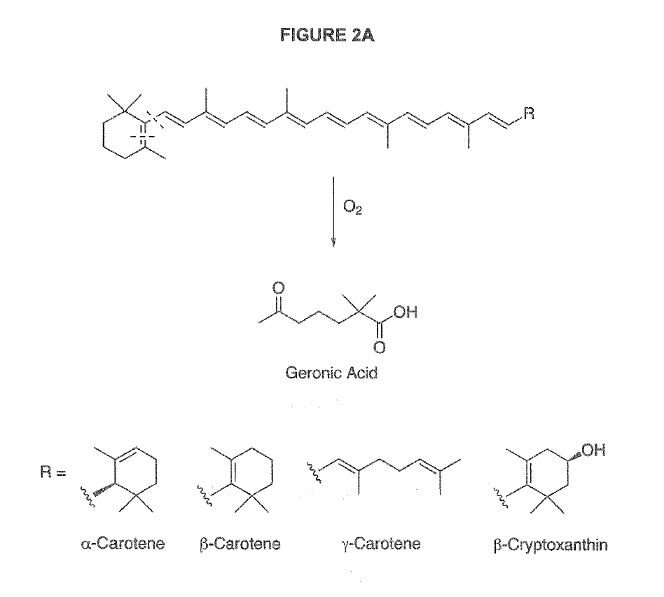

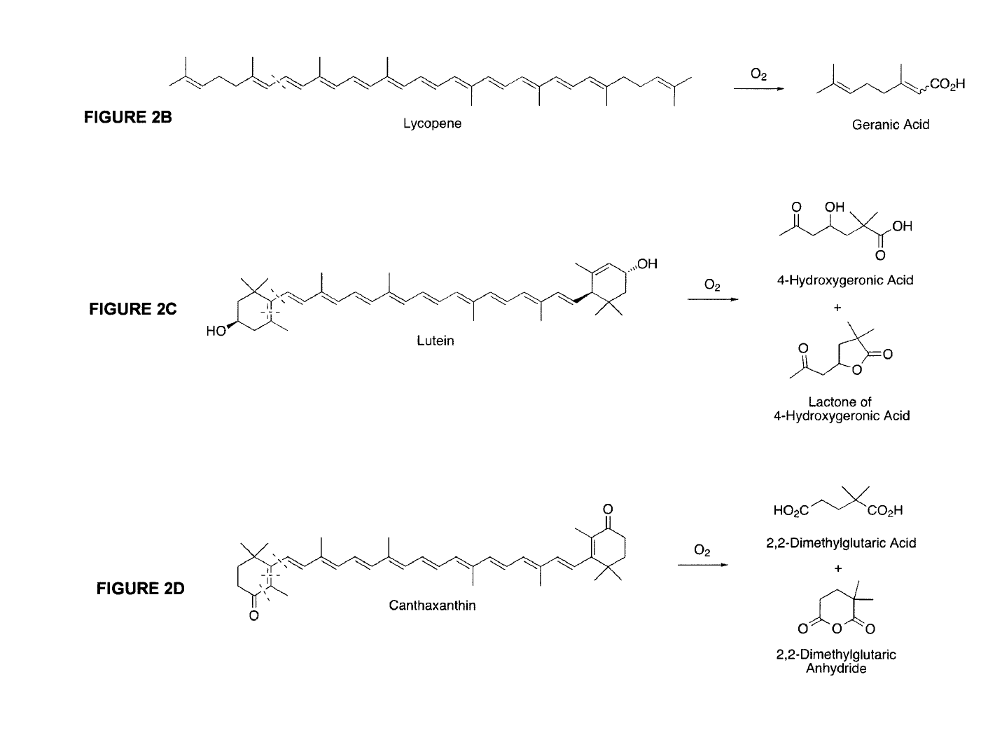

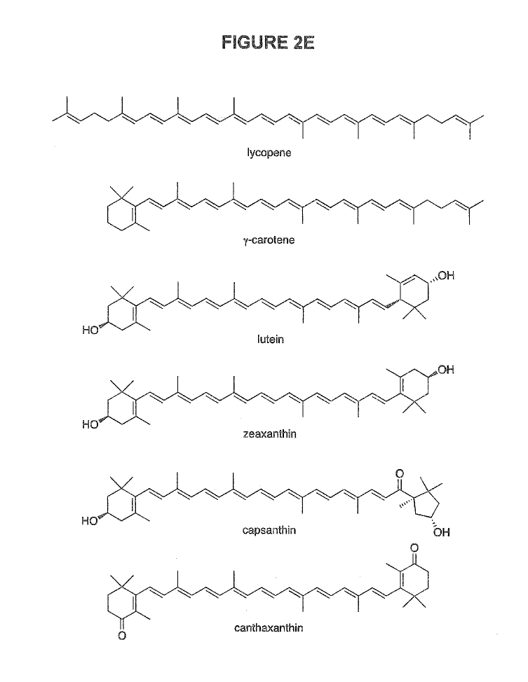

Because carotenoid-oxygen copolymers in a food matrix are not readily amenable to any direct chemical or biochemical measurement, the inventors developed a novel indirect approach that used indicators/markers to determine the extent of oxidation and polymer formation. These are illustrated in FIG. 2, where: (i) geronic acid is a marker for the carotenoid-oxygen copolymers of β-cryptoxanthin; α-carotene; β-carotene, and γ-carotene; (ii) geranic acid is a marker for the carotenoid-oxygen copolymers of lycopene and γ-carotene; (iii) 4-hydoxygeronic acid and/or its lactone are markers for the carotenoid-oxygen copolymers of lutein, zeaxanthin, and capsanthin; and (iv) 2,2-dimethylglutaric acid and its anhydride are markers for the carotenoid-oxygen copolymer of canthaxanthin.

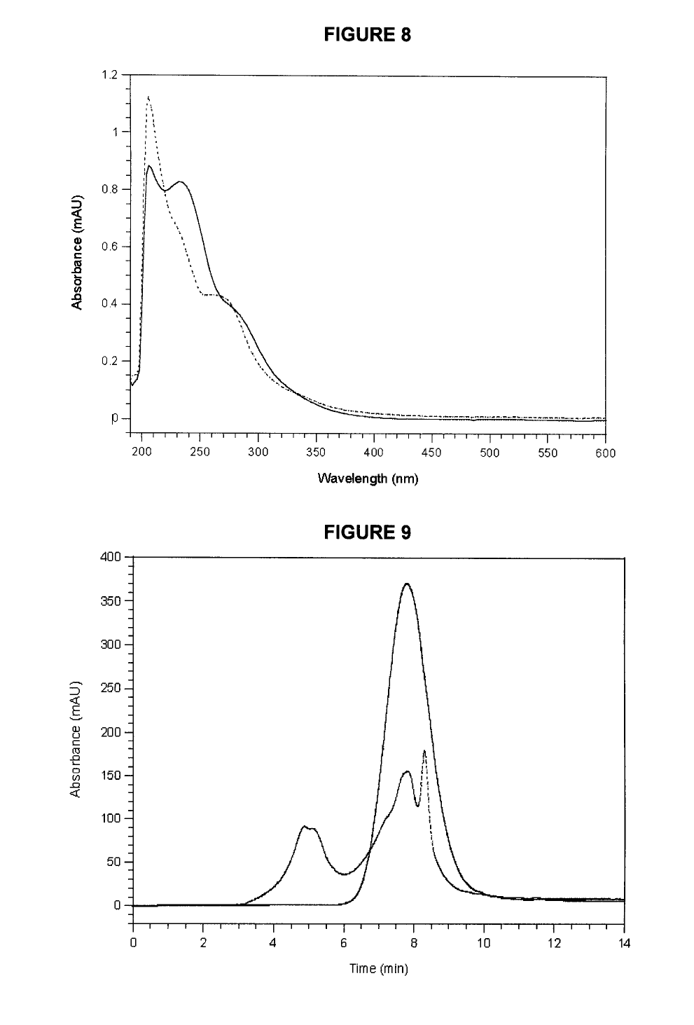

Taking geronic acid as an example, one can quantify the presence of the carotenoid-oxygen copolymer. While copolymer product dominates throughout the course of the model oxidation (≥80%), corresponding to eventual uptake of almost 8 molar equivalents of oxygen, GA, the most abundant norisoprenoid product,¹³ is formed continuously at 1-3% of the total reaction product weight (see FIG. 8 in ref. 13). Taking the average value for GA to be about 2% of the total product weight, the amount of oxidation products therefore can be estimated to be roughly ˜50 times larger, which, given the dominance of the copolymer, translates into an ˜50:1 polymer:GA ratio. However, the actual ratio could lie between 25:1 to 100:1 given its approximate nature.

The finding of the present inventors that oxidation and the associated reaction products would be found within the much more complex environment in which carotenoids occur naturally, namely in certain plant sources, such as fruits and vegetables and certain microorganisms (algae, fungi and bacteria) was not obvious or predictable in light of the complex micro-environment and the many other potentially reactive compounds in the biological material that could divert any incipient carotenoid oxidation reaction down a myriad of other pathways with different product outcomes.

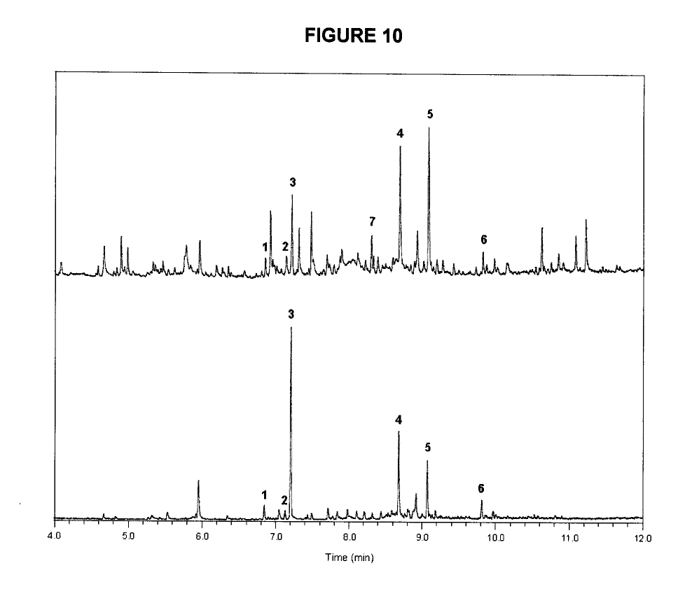

The carotenoid-oxygen copolymer product(s) of the present invention isolated from such natural sources is not the same as OxBC or the products obtained from full oxidation of other pure carotenoids (e.g., OxLyc, OxLut or OxCan from lycopene, lutein and canthaxanthin, respectively) because the latter comprise the low molecular weight compounds as well (which are herein removed by the isolation process for the polymer from the food-derived product). In some embodiments, the natural source product often also still comprise one or more unreacted carotenoids. For instance, natural source product may also comprise other compounds that get incorporated during the polymerization reaction, as illustrated in FIG. 10 showing the carrot powder GCMS thermolysis chromatogram compared to OxBC derived from pure carotenoids. Such in some embodiments, the present invention provides an OxPVA or OxCAR composition which comprise the respective carotenoid-oxygen copolymer components derived from natural sources. In some embodiments said compositions do not comprise norisoprenoid by-products.

In another embodiment, the invention provides a method to prepare products from natural sources that comprise carotenoid-oxygen copolymers. In one embodiment, the method comprises using GRAS solvents. In another embodiment the method comprises extracting a dried food source with ethyl acetate, a GRAS solvent, which step will dissolve most if not all β-carotene-oxygen copolymers, and then to slowly precipitate the copolymer compound free of other more soluble compounds with careful addition of a non-polar solvent. In general the extraction/precipitation process of the invention requires a minimum amount of solvent that dissolves the polymer and then adding a non-polar solvent, for instance, dropwise to cause the polymer to precipitate out of solution and then collecting it by filtration or centrifugation. In one aspect of the invention, the carotenoid-oxygen copolymer products isolated in this manner from dried plant-derived foods do not contain the other anticipated low molecular weight carotenoid breakdown products (e.g., including the indicators noted above such as geronic acid in products expected to contain β-carotene oxidation breakdown compounds). This is distinct from fully oxidized carotenoids (such as, OxBC, OxLyc, OxLut or OxCan), derived from pure carotenoid sources which, without subsequent solvent precipitation purification, do comprise such products.

An Indirect Method for Assessing the Presence of Carotenoid-Oxygen Copolymers. General

The present invention discloses a use of an indirect, low molecular weight marker of oxidative polymerization of carotenoids, such as provitamin A carotenoids, e.g., geronic acid at ˜2% of β-carotene copolymers, can be used to assess the amount of the carotenoid-oxygen copolymers in a potential source of same. The invention also discloses that other low molecular weight markers could be used as indicators of oxidative polymerization of other selected carotenoids, including lycopene, lutein, zeaxanthin and canthaxanthin.

GA has been measured in a variety of foods, ranging from fresh foods, e.g., carrot juice and raw tomatoes, in which oxidation is expected to be minimal, to foods dried by processes likely to cause adventitious oxidation, including increasing the partial pressure of oxygen (ppO₂) or temperature, dehydration, grinding, powdering and exposure to light. The GA determination is a useful guide to isolating carotenoid-oxygen copolymer compounds in identified GA-rich foods and food sources (dried) with high geronic acid levels were chosen as candidates for extraction and isolation of solid oxygen copolymer compounds. That is, carotenoid oxidation was taking place within natural sources. Further, it was found that levels of geronic acid were much higher in food sources subjected to processes that increased exposure to oxygen through drying and that increased affected surface area. This is similar to the other carotenoid-oxygen co-polymers and their indicators.

### The Method

Herein, the inventors have shown that geronic acid and carotenoid-oxygen copolymer products occur naturally in plant-based foods containing carotenoids, such as provitamin A carotenoids, especially in processed products. Further, the inventors herein have shown that GA is a specific indicator of oxidation of β-carotene and other provitamin A carotenoids in these foods. There are few previous reports of the natural occurrence of GA.^{25, 26} Although GA can be made in the laboratory by oxidation of certain norisoprenoid compounds, e.g., 1-cyclocitral, in plants these compounds are themselves likely to originate from carotenoid oxidation. In animal-derived products, however, it is possible that GA can come from several sources, including the diet and from oxidation of vitamin A. Similarly, the inventors have made similar findings regarding the utility of low molecular weight indicators for the presence of other carotenoid-oxygen copolymers.

Using the oxidation of β-carotene¹³ as a model, the inventors herein have developed a method to correlate GA in foods with β-carotene-oxygen copolymer formation. Substantial quantities of carotenoid-oxygen copolymers were isolated from carrot powders, which had the highest concentrations of GA of all foods examined. Carrot powder #1 had double the GA of carrot powder #2 and yielded almost double the amount of copolymer. The chemical identity of the compounds isolated from the carrot powder extracts is established by the combined evidence from GPC, elemental analysis, IR and UV-Vis spectroscopies, and GC-MS thermolysis, which points strongly to a predominance of β-carotene-oxygen copolymers.

Although GA may be used as indirect marker for provitamin A carotenoid-oxygen copolymers whose parent carotenoids have β-ionone ring groups, including α-carotene, β-carotene, γ-carotene, and β-cryptoxanthin, other indirect markers can be used for the same or other carotenoids such as lutein, zeaxanthin, capsanthin, lycopene, γ-carotene, or canthaxanthin. For example, in one embodiment 4-hydroxygeronic acid or its lactone can be used as the indirect marker for lutein, zeaxanthin or capsanthin. In another embodiment geranic acid can be used as an indirect marker for lycopene or γ-carotene. In another embodiment 2,2-dimethylglutaric acid or its anhydride can be used as an indirect marker for canthaxanthin. In other embodiments, esters (such as methyl esters) and or labeled forms (such as deuterium-labeled) of these markers can be used to facilitate chemical analysis.

As such, in some embodiments, the invention provides a method for determining the presence of carotenoid-oxygen copolymer in a source comprising:

(a) oxidizing a pure carotenoid that is known to be present in the source and determining the ratio of the resulting carotenoid-oxygen copolymer (addition product) to an indicator (cleavage product of the reaction) and creating a calibration curve of carotenoid-oxygen copolymer to cleavage product under one or more conditions selected from: time, temperature, pressure, source, amount of starting material and exposure to oxygen;

(b) processing the source under oxidizing conditions and identifying and or quantifying the amount of resulting indicator (cleavage product of the reaction); and

(c) using the results in (b) and the calibration curve developed under (a) to identify the presence or lack thereof of carotenoid-oxygen copolymer and/or to determine the amount of resulting carotenoid-oxygen copolymer in the source.

In some embodiments, sources that comprise 1-1000 parts per million (ppm) wet weight or 10-10,000 ppm dry weight of carotenoids, which may translate upon full oxidation to similar levels of carotenoid-oxygen copolymers are selected.

In some embodiments, the carotenoid has a β-ionone ring. In another embodiment, the carotenoid is selected from a group consisting of: α-carotene, γ-carotene, β-carotene, and β-cryptoxanthin. In some embodiments, if the carotenoid has a β-ionone ring group, the indicator is geronic acid. In some other embodiments, the carotenoid is selected from the group consisting of lutein, zeaxanthin, capsanthin, lycopene, γ-carotene, and canthaxanthin, and their respective indicators are as noted above.

In some other embodiment, the source is selected from the group consisting of: carrots, tomatoes, alfalfa, spirulina, rosehip, sweet pepper, chili pepper, paprika, sweet potato, kale, spinach, seaweed, wheatgrass, marigold⁴⁵⁻⁴⁸, moringa oleifera⁴⁹⁻⁵² and red palm oil. In another embodiment, the source is in powder form. In another embodiment, the source is tomato pomace. In another embodiment, the source is a microorganism.

### Isolation of Carotenoid-Oxygen Copolymer Products

Large amounts of carotenoid-oxygen copolymers also were isolated from other dried foods in which carotenoids other than β-carotene are abundant (e.g., lycopene, lutein and capsanthin). These foods include tomato powder, rosehip powder, paprika, sun-cured alfalfa and wheatgrass powder.

It is expected that the makeup of the polymeric compounds is modified to some extent by the environment in which they are formed. The adventitious nature of the oxidation, the complexity and variety of reaction sites and the presence of other reactive compounds will result in a variable product, unlike in the highly reproducible oxidation of pure, individual carotenoids in a homogenous organic solvent (e.g., β-carotene, lycopene, lutein, canthaxanthin).

The molecular weight profiles from the GPCs of the products isolated from foods indeed show complexity compared to those from the oxidations of the individual representative carotenoids. The empirical formulae of most of the food compounds show more hydrogen is present than in the copolymers obtained from oxidation of individual carotenoids, suggesting the presence of small amounts of some compounds comprising saturated hydrocarbon components. Also, minor amounts of nitrogen-containing components are present, and thermolysis of the carrot extract yields more unknown breakdown products than does the OxBC polymer. The IR spectra, however, show a very striking degree of similarity across all compounds.

In several dried foods the level of copolymers is comparable to the original level of the parent carotenoid, e.g., in carrot and tomato powders.

As such, in one embodiment, the invention provides a method of isolating additional oxygenated carotenoid products from a source that comprises carotenoids.

In some embodiments, the purity and amount of carotenoid-oxygen copolymer can be adjusted to desired levels by a number of extraction/precipitation cycles during processing. In one embodiment, the invention provides a method of producing compositions with consistent and desired amounts of carotenoid-oxygen copolymers by being able to select sources with known levels of carotenoids to, through oxidation, produce products with known levels of carotenoid-oxygen copolymers and/or adding known amounts of carotenoid-oxygen copolymers or compositions comprising known amounts of same with desired other excipients or foods, for instance as a supplement with known amounts of carotenoid-oxygen copolymer, incorporated into or as a supplement to animal feed or incorporating into or as a supplement into human food or supplement sources, including but not limited to spices, breads, processed meat products, soups and other foods.

### Uses of Carotenoid-Oxygen Copolymers

The inventors' discovery that OxBC (β-carotene-oxygen copolymer) compounds have beneficial, non-vitamin A immunological activities^{14,16} leads to the expectation that carotenoid-oxygen copolymer counterparts in foods will impart bioactivities with significant health implications. OxBC has demonstrated health benefits at parts-per-million dietary levels in swine²⁷, poultry, canines and fish. In humans, carotenoid-oxygen copolymers could contribute to the beneficial health effects associated with fruit and vegetable consumption.⁵ In situ oxidation of dietary carotenoids resulting from oxidative processes unleashed during digestion of fruit or vegetables also could at least partially account for the variable and several-fold lower vitamin A activity of β-carotene in foods compared to β-carotene from supplements.^{4, 28} Oxidative destruction of β-carotene and a perceived loss of activity could actually be a gain of immunological activity through copolymer formation.

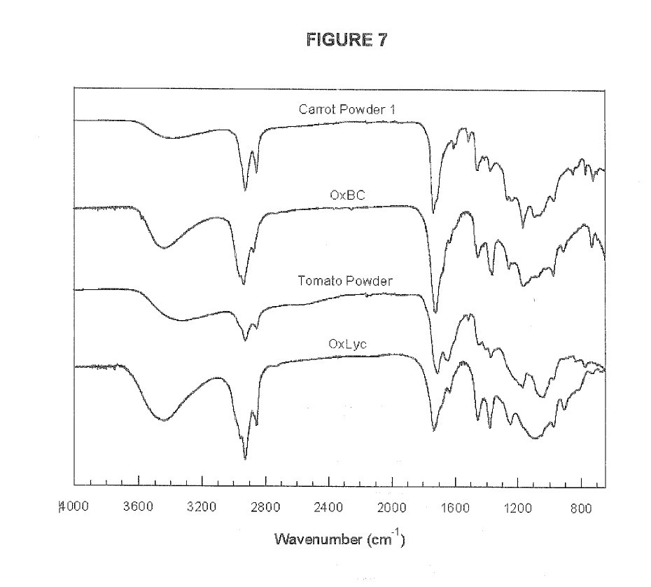

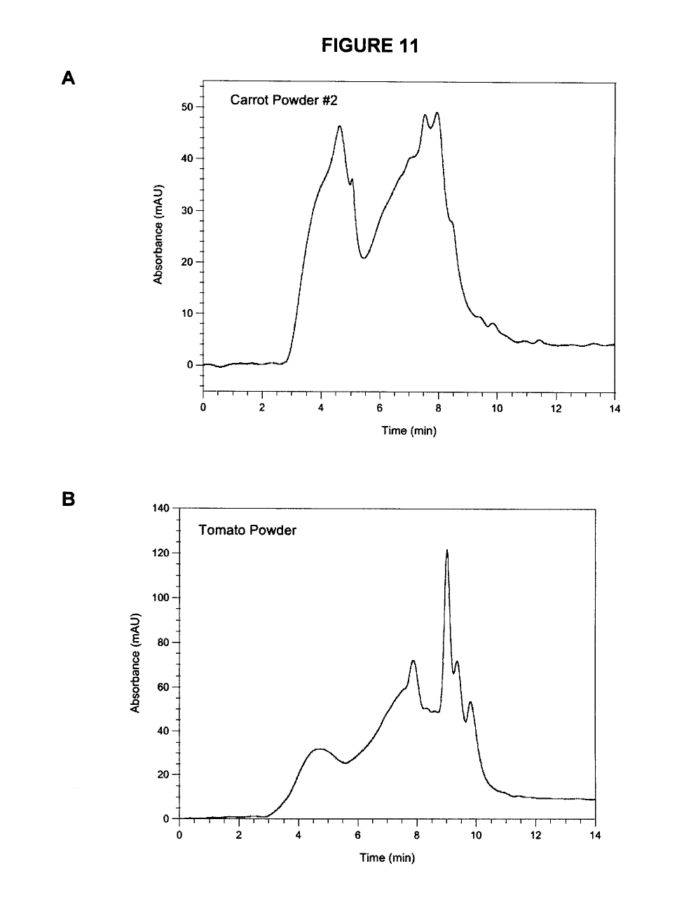

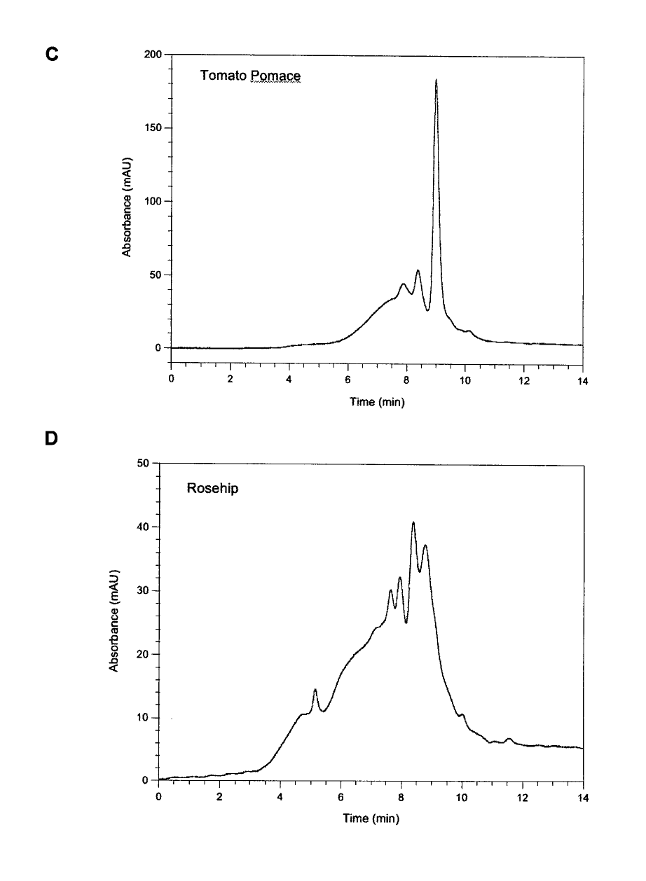

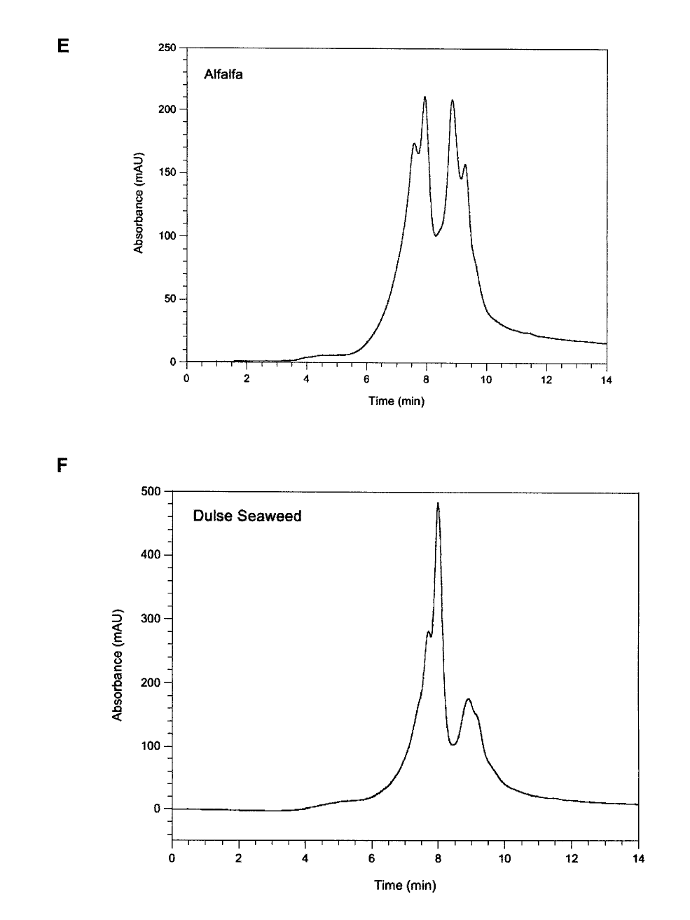

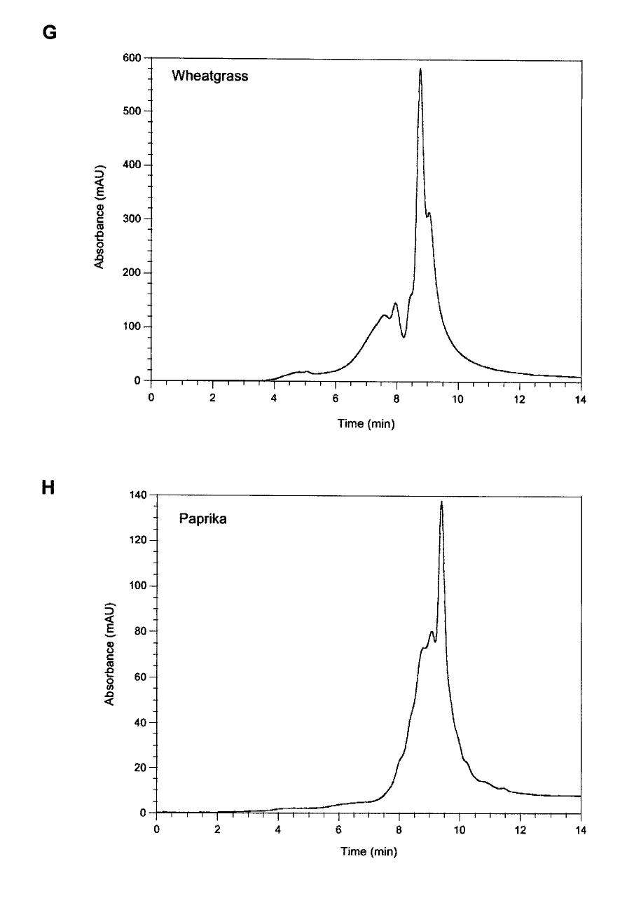

In noting that lycopene is even more susceptible than β-carotene to formation of active copolymer products,^{13,14} it is likely lycopene-oxygen copolymer formation accompanies the significant losses of lycopene that occur during tomato processing.²⁹ In a rat model of prostate carcinogenesis, tomato powder but not lycopene alone inhibited carcinogenesis.³⁰ The authors concluded that this finding suggests, “tomato products contain compounds in addition to lycopene that modify prostate carcinogenesis”. Lycopene-oxygen copolymers are present in tomato powder, as documented here in the tables and figures (Tables 3 and 4 and FIGS. 7 and 11) and it is likely they are present in other processed tomato products.

The extended system of linear conjugated double bonds present in β-carotene is common to all carotenoids so it is expected that other carotenoids will behave similarly in their spontaneous reactions with molecular oxygen and may explain the non-vitamin A effects of both the provitamin A carotenoids (α-, β- and γ-carotenes and β-cryptoxanthin) and the more numerous carotenoids that cannot be converted into vitamin A.

The present invention in some aspects enables one to enhance the amount of carotenoid-oxygen copolymer in a source and/or to have a source with known and consistent amounts of carotenoid-oxygen copolymer to facilitate consistent dosing to known effective amounts to achieve desired results, such as the enhancement of overall health and immunity as described above.

Further, the present invention in some aspects enables one to produce carotenoid-oxygen copolymers comprising products in situ without starting from isolated carotenoids as the source and to provide products comprising consistent levels of carotenoid-oxygen copolymers which have resulting animal and human health benefits.

### Examples

### Example 1—Materials and Methods

Materials

The preparations of GA, GA-d₆, and fully oxidized β-carotene (OxBC), lycopene (OxLyc) and canthaxanthin (OxCan) have been described.13 For this study fully oxidized carotenoids, including fully oxidized lutein (OxLut), were prepared at 68-70° C. as noted below. SPE cartridges were obtained from Waters (Oasis MAX; 500 mg sorbent, 6 mL capacity). Silica gel (40-63 μm) was purchased from Silicycle Inc. (Quebec City, QC Canada) and silica gel TLC plates were purchased from Sigma-Aldrich.

### Equipment.

GC-MS was performed with an Agilent Technologies 6890N GC with a 5975B VL mass selective detector. The GC was equipped with an HP 5 column, 30 m×0.25 mm×0.25 μm. Measurement conditions: initial pressure 17 psi, constant flow of 1.0 mL/min; injector temperature 250° C.; initial oven temperature 50° C. for 1 min, temperature ramp 20° C./min to 280° C., hold time 2.5 min. The instrument was used in SIM mode to monitor ions m/z=154 and 160. (Note: for tomato powder and red palm oil, two different temperature programs were used. Program 1 (tomato powder): start 50° C., ramp 8° C./min until 210° C. then ramp 20° C./min until 280° C. and hold 2.5 min. Program 2 (red palm oil): start 50° C., ramp 10° C./min until 210° C., then ramp 20° C./min until 280° C. and hold 2.5 min.)

FTIR spectra for OxBC and OxLyc were obtained with a Varian 660-IR spectrometer using KBr pellets or NaCl disks and film casts from chloroform solutions of samples (one drop of ca. 50 mg/mL). FTIR spectra of all other samples were obtained using a Thermo 6700 FTIR spectrometer with Smart iTR accessory for attenuated total reflectance (diamond surface).

GPC chromatograms were obtained using an HP 1090 HPLC apparatus equipped with a diode array detector and a 7.8×300 mm Jordi Flash Gel 500A GPC column (5 μm particle size; Jordi Labs LLC, Bellingham, Mass. 02019 USA). Samples were dissolved in and eluted with THF at 1 mL/min for 14 min.

UV-Vis spectra were recorded in methanol with a Hewlett Packard 8452 Diode Array Spectrophotometer using a 1 cm path length quartz cell.

Elemental analyses were performed by Canadian Microanalytical Service Ltd., Delta, BC, Canada.

### Food Samples

Unless noted otherwise, all samples were purchased locally (Ottawa, Ontario, Canada). Carrot juice, dried dates, homogenized milk (3.25% milk fat), whole milk powder (3.25% milk fat) and yellow corn flour were bought at grocery stores. Fresh red tomatoes were purchased at a farmers' market. Sun-cured alfalfa was bought at a pet store and spirulina powder was purchased from a health food store. Carrot powder #1, paprika and echinacea purpurea root powders were purchased from Monterey Bay Spice Co. (Watsonville, Calif.). Rosehip powder was purchased from Coesam S.A. (Santiago, Chile) and cranberry powder was purchased from Atoka Cranberries Inc. (Manseau, Quebec). Honey and bee pollen were purchased from Dutchman's Gold Inc. (Carlisle, Ontario). Carrot powder #2 (air dried), tomato powder (air dried), sweet potato powders #1 and #2 (air dried and drum dried, respectively) were bought from North Bay Trading Co. (Brule, Wis.). Tomato pomace was obtained from LaBudde Group Inc. (Grafton, Wis.). Dulse seaweed powder was purchased from Z Natural Foods (West Palm Beach, Fla.), and nori seaweed flakes were obtained from Global Maxlink Inc. (Antelope, Calif.). Red palm oil was purchased from Well.ca (Guelph, Ontario). Whole egg powder and wheatgrass powder were bought from Bulkfoods.com (Toledo, Ohio). Brown rice flour was purchased from Yupik.ca (Montreal, QC).

### Preparation of Fully Oxidized Carotenoids at 68-70° C.

Materials.

β-carotene, lycopene, lutein and canthaxanthin were obtained from Allied Biotech Corp (Taiwan).

Preparation of OxBC.

β-carotene (80 g) was placed in a 3-neck flask with stir bar, reflux condenser, O₂ inlet (glass tube), and a temperature probe connected to a heating mantle to monitor and adjust the temperature as needed. Ethyl acetate (2 L) was added, O₂ was bubbled through and the mixture was stirred and heated to 68° C. After 66 h, the absorbance of a sample was measured as 0.36 at 380 nm using a 1 mm cuvette at ca.10 g/L, indicating the reaction was complete¹³. The reaction was stopped and the clear, light orange liquid was cooled to room temp and split between two 1-L round bottom flasks. Solvent was removed on the rotary evaporator at 40° C. down to a pressure of 30 mm Hg, and the resulting syrup was dried under vacuum for 10 h to give a sticky orange solid (106.9 g) as OxBC.

Preparation of OxLye.

Lycopene (817 mg) was placed in a 3-neck flask with stir bar, reflux condenser and O₂ inlet (Pasteur pipette). Ethyl acetate (20.4 mL) was added, O₂ was bubbled through and the stirred mixture was lowered into an oil bath at 68° C. After 21 h, the reaction was stopped and the slightly cloudy yellow liquid was cooled to room temp. The absorbance of a filtered sample was measured as 0.272 at 380 nm in a 1 cm cuvette at ca. 1.0 g/L. The cloudy yellow liquid was centrifuged, the supernatant decanted and the solid residue rinsed/centrifuged with ethyl acetate (2×3 mL), decanting the supernatant each time. The solid residue was dried under vacuum for 4 h to give a pale yellow, flaky solid (38 mg). The combined supernatant liquids were transferred to a 50 mL round bottom flask and solvent was removed on the rotary evaporator at 40° C. down to a pressure of 30 mm Hg. The residue was transferred to a vacuum pump and dried for 4 h to give a foam-like, yellow solid (1.036 g) as OxLyc.

Preparation of OxLut.

Lutein (1.00 g) was placed in a 3-neck flask with stir bar, reflux condenser and O₂ inlet (Pasteur pipette). Ethyl acetate (25 mL) was added, O₂ was bubbled through and the stirred mixture was lowered into an oil bath at 70° C. After 18 h 45 min, precipitate was observed in the yellow solution and the reaction was stopped and cooled to room temp. After cooling, more precipitate came out of the solution. The absorbance of a filtered sample was measured as 0.0712 at 380 nm in a 1 cm cuvette at ca. 0.4 g/L. The mixture was centrifuged and the supernatant filtered through a 0.45 μm Teflon syringe filter to remove a few fine flakes. The solid fraction was rinsed with ethyl acetate (2×3 mL) and centrifuged, decanting the liquid each time. The solid was dried under vacuum to give a light brown powder (105.8 mg). The liquid fractions were combined and solvent evaporated at 40° C. down to a pressure of 30 mm Hg, followed by drying on the vacuum pump for 3 h to give a yellow, foam-like solid (1.143 g) as OxLut.

Preparation of OxCan.

Canthaxanthin (1.0021 g) was placed in a 3-neck flask with stir bar, reflux condenser and O₂ inlet (Pasteur pipette). Ethyl acetate (25 mL) was added, O₂ was bubbled through and the stirred mixture was lowered into an oil bath at 69° C. After 71 h, the reaction was stopped and cooled to room temp. The absorbance of the clear yellow solution was measured as 0.6308 at 380 nm in a 1 cm cuvette at ca. 0.5 g/L. The solution was transferred to a 50 mL round bottom flask and solvent was removed on the rotary evaporator at 40° C. down to a pressure of 30 mmHg, then dried carefully on a vacuum pump, allowing the solid material to expand just enough to fill the flask. After 2 h 45 min under vacuum, a foam-like, yellow solid was obtained (1.250 g) as OxCan.

Isolation of Carotenoid-Oxygen Copolymers from Fully Oxidized Carotenoid Compounds Obtained from Pure Carotenoids

OxBC Polymer.

OxBC (2.05 g) was dissolved in ethyl acetate (5 mL) and hexanes (50 mL) were added dropwise with stirring. The liquid was decanted from the precipitated solid and the latter dissolved in a minimum of ethyl acetate. Solvent was removed on the rotary evaporator at 40° C. down to a pressure of 20 mm Hg, then the liquid concentrate dried on a vacuum pump for 1 h to give a solid. The obtained solid was precipitated twice more as above to give OxBC polymer as a yellow-orange solid (1.076 g).

OxLyc Polymer.

OxLyc (826 mg) was dissolved in ethyl acetate (1 mL) and hexanes (50 mL) were added dropwise with stirring. One hour after complete addition, the liquid was decanted from the precipitated solid, which was then rinsed with hexanes (3×3 mL). The residue was dried on the vacuum pump for 1 h, and the precipitation repeated twice more using ethyl acetate/hexanes (1 mL/25 mL, then 1 mL/10 mL). The solid material was dissolved in a minimum of ethyl acetate then dried on the vacuum pump for 3.5 h to give OxLyc polymer as a yellow solid (700 mg).

OxLut Polymer.

To OxLut (836 mg) was added 3:2 ethyl acetate:methanol (5 mL) and the mixture was almost completely dissolved (some fine flakes were present). Hexanes (50 mL) were added dropwise with stirring and after several mL were added the cloudy mixture became clear, dissolving completely. Addition of the remaining hexanes caused some material to precipitate. After complete addition, the mixture was stirred 15 min and a clear yellow liquid was visible on top of a thick, yellowish orange syrup. The yellow liquid was decanted and the syrup rinsed 7×3 mL hexanes. The syrup was dissolved in ethyl acetate (3 mL), solvent removed on the rotary evaporator to 60 mmHg at 40° C., and the residue dried on the vacuum pump for 1.5 h to give a brittle yellow solid (585 mg). The solid was dissolved in ethyl acetate (1 mL) and hexanes (10 mL) were added dropwise with stirring. After 30 min, the liquid was decanted and the residue rinsed with hexanes (5×1.5 mL). The residue was dissolved in ethyl acetate (4 mL), then solvent was removed on the rotary evaporator at 35° C. down to a pressure of 45 mmHg and the residue dried on the vacuum pump for 45 min to give a brittle yellow solid (567 mg). The precipitation was repeated once more using ethyl acetate (1 mL) and hexanes (10 mL) and rinsing with hexanes (5×1.5 mL). The residue was dried on the vacuum pump for 2.5 h to give OxLut polymer as a brittle yellow solid (565 mg).

OxCan Polymer.

OxCan (796 mg) was dissolved in ethyl acetate (1 mL) and hexanes (10 mL) were added dropwise with stirring. One hour after complete addition, the liquid was decanted and the residue rinsed with hexanes (3×1.5 mL). The residue was dried on the vacuum pump for 1 h, and the precipitation repeated twice more as above. The solid was dissolved in a minimum of ethyl acetate then dried on the vacuum pump for 2 h to give OxCan polymer as a yellow solid (646 mg).

### Example 2—Analysis of Geronic Acid in Food Samples and Use of Same to Estimate Provitamin A Carotenoid-Oxygen Copolymer Content

General Extraction Procedure.

To minimize adventitious oxidation of carotenoids during extraction, all organic solvents contained 0.1% BHT or, alternatively, an equivalent amount was added to the sample immediately prior to extraction. Food samples were homogenized in aqueous organic solvent mixtures with either chloroform for raw foods or aqueous acetonitrile for dry foods immediately prior to extraction. Extractions were carried out as follows: 1) add GA-d₆ standard to the aqueous suspension of sample and extract multiple times with chloroform or blend multiple times with acetonitrile and filter; 2) combine and concentrate the extracts, mix the concentrate with chloroform and magnesium or sodium sulfate, filter and treat the filtrate with aqueous KOH to extract carboxylic acids (2×); 3) acidify the combined aqueous KOH extract with aqueous HCl to isolate carboxylic acids and extract into chloroform or dichloromethane; 4) dry and evaporate the separated chloroform or dichloromethane fraction; and 5) esterify the residue with trimethyloxonium tetrafluoroborate according to the following procedure.

Esterification of Extract with Trimethyloxonium Tetrafluoroborate.

After evaporation of the solvent under a stream of nitrogen or by rotary evaporation, the residue was dissolved in methanol (4.5 mL). Aqueous sodium bicarbonate solution (1 M, 1 mL) was added followed by trimethyloxonium tetrafluoroborate (ca. 0.3 g) in small portions over 1-5 min (pH maintained weakly basic by addition of solid sodium bicarbonate). The resulting mixture was stirred 10 min at room temperature, then water added (4-9 mL) and the product extracted with dichloromethane (2×9 mL). The combined dichloromethane extracts were dried over magnesium sulfate, filtered, and the solvent evaporated to provide the methyl esters, which were taken up into acetonitrile and filtered for GC-MS analysis.

Detailed Extraction Procedures.

Descriptions for carrot juice, carrot powders, raw tomato, tomato powder, tomato pomace, dates, milk, milk powder, whole egg powder, raw cranberry, cranberry powder, rosehip powder, spirulina powder, paprika powder, sweet potato powders, dulse powder, nori flakes, sun-cured alfalfa, wheatgrass powder and red palm oil are provided below.

Carrot Juice.

Purification and concentration of GA was achieved using chloroform. The chloroform extract contained a complex mixture of substances, including carotenoids and carboxylic acids. Acids present in the fraction were extracted into basic aqueous solution (pH 12-13; GA is soluble in water at pH 12-13) and recovered by acidification of the extract followed by re-extraction into chloroform. Attempts to use anion exchange SPE cartridges failed to concentrate and purify GA.

GA-d₆ (2.9 μg in 0.5 mL methanol) was added to carrot juice (ca. 200 g) and mixed with chloroform (250 mL; 0.1% BHT). After vigorous stirring for 2.5 h the emulsion was transferred to a separatory funnel and the chloroform layer was separated. The aqueous fraction was extracted again with chloroform (250 mL; 0.1% BHT) and the chloroform extracts were combined, dried over MgSO₄, and filtered through celite. The celite and filter were washed with chloroform (50 mL) and the washing added to the combined chloroform solutions. The combined extracts and washing gave a clear solution that was concentrated to ca. 100 mL by rotary evaporation. Carboxylic acids present in the extract were extracted by stirring vigorously for 15 min with aqueous KOH (0.032 M; 2×100 mL), acidifying the combined aqueous extracts (5% aqueous HCl) to pH 2.5 and extracting the acids into chloroform (2×100 mL). The solvent was removed from the combined chloroform extracts by rotary evaporation and the residue esterified (conversion to methyl ester according to the procedure described in paragraph [0096]).

Supporting evidence for the presence of geronic acid in carrot juice was obtained by converting the methyl ester into its corresponding semicarbazone derivative via the keto functionality. The GA methyl ester was regenerated from the isolated semicarbazone fraction and analyzed by GC-MS.

The mixture of esters obtained above was dissolved in 3:1 methanol:water (4.5 mL). Solid semicarbazide hydrochloride (ca. 0.15 g) was added, followed by solid sodium acetate (ca. 0.15 g). The resulting suspension was briefly heated to reflux, cooled to room temperature, extracted with dichloromethane (2×9 mL) and the solvent evaporated. The residue was extracted with chloroform (5×1.5 mL) and the combined extracts were passed through a short pad of silica gel (6 mL). The silica was eluted with chloroform (20 mL) and semicarbazones eluted with methanol (15 mL). The methanol was evaporated, the residue dissolved in chloroform (9 mL) and activated MnO₂ (0.3 g) added. The suspension was vigorously stirred for 1 h at room temperature, filtered and the filtered MnO₂ washed with chloroform (2×1.5 mL). The chloroform fractions were combined and the solvent removed by rotary evaporation. The residue was dissolved in acetonitrile (0.6 mL), filtered and the solution analyzed by GC-MS.

Carrot Powders #1 and #2.

Distilled water (25-30 mL), acetonitrile (125-150 mL), BHT (ca. 5-9 mg) and GA-d₆ (15 or 30 μg in methanol) were added to carrot powder (ca. 5.0 g) in a 400 mL beaker. The mixture was homogenized for 20-25 min at 9,500 rpm, then filtered through a sintered glass Buchner funnel. The filtrate was set aside and the solid residue homogenized again as described with acetonitrile/water (125-150 mL/25-30 mL). The mixture was filtered and both filtrates were combined. The solvent was evaporated and the oily residue re-dissolved in chloroform (20 mL), dried (Na₂SO₄; note that over several minutes an orange liquid coalesces and floats on top of the chloroform, then becomes adsorbed onto the drying agent), and filtered through cotton. The flask and filter were rinsed with chloroform (4×5 mL) and the rinsings combined with the filtrate. The chloroform was stirred vigorously for 5 min with aqueous KOH (˜0.03 M; 25 mL), then allowed to settle. A separatory funnel was used to remove the bulk of the chloroform, and the remainder was removed by centrifugation. The aqueous layer was set aside and the chloroform layer extracted as above with aqueous KOH (0.03 M; 25 mL). The aqueous layers were combined, acidified with 1 M HCl to pH 1-2 and extracted with chloroform (2×20 mL). The combined chloroform extracts were dried (Na₂SO₄), filtered through cotton, the solvent evaporated and the residue esterified.

Raw Tomato.

GA-d₆ (2.8 μg in methanol), aqueous HCl (1.5 mL, 3.6% v/v) and chloroform (250 mL; 0.1% BHT) were added to fresh tomatoes (200-250 g) that had been chopped into small pieces. The mixture was homogenized and the resulting paste transferred to a glass-stoppered flask, rinsing with chloroform to ensure complete transfer. After standing at 2° C. overnight in the dark the suspension formed two liquid phases. Excess MgSO₄/NaCl (1:1) was added to the separated, bottom chloroform layer. A fine suspension formed and the mixture was filtered through celite. The celite and filter were washed with chloroform and the combined chloroform solutions were concentrated to ca. 60 mL. Carboxylic acids were extracted by vigorously stirring the concentrate for 8 min with aqueous KOH (0.1 M; 2×65 mL). The combined aqueous extracts containing a yellow suspension were washed with dichloromethane/ethyl acetate (2:1, 60 mL) and acidified (5% aqueous HCl) to pH 2.3. Carboxylic acids were extracted with dichloromethane (2×50 mL) and the extract dried (MgSO₄) and filtered. The solvent was removed by rotary evaporation and the residue esterified.

Tomato Powder. To ca. 5.0 g tomato powder was added BHT (3-4 mg), distilled water (30 mL), acetonitrile (150 mL) and geronic acid-d₆ (6.2 μg in methanol). It was homogenized at 9500 RPM for 25 min, then filtered through a sintered glass Buchner funnel. The solid residue was extracted once more as above with acetonitrile/water (150 mL/30 mL), the filtrates were combined and solvents evaporated, leaving ˜2-3 mL oil. Chloroform (15 mL) was added and the mixture was dried (Na₂SO₄) and filtered through cotton, rinsing with chloroform (4×4.5 mL). The combined filtrates were stirred vigorously for 5 min with aqueous KOH (˜0.03 M; 25 mL). Most of the chloroform was removed by separatory funnel and the remainder was separated by centrifuge. The aqueous layer was collected and the chloroform layer extracted once more with aqueous KOH as above. The aqueous layers were combined, acidified with 1 M HCl (˜3 mL) and extracted with chloroform (2×20 mL). The combined chloroform extracts were dried (Na₂SO₄), filtered and concentrated to 0.3-0.5 mL. The liquid was transferred to a column of dry silica gel (2 cm diameter, 13.5 cm length) and N₂ passed through. After the silica was dry, the column was eluted using methanol:ethyl acetate:hexanes (5:10:85) and N₂ pressure, collecting fractions of ca. 10 mL every ˜1.5 min. Fractions 18-25 (containing a yellow band and co-spotting with geronic acid) were combined, solvents evaporated and the residue esterified.

Tomato Pomace.

To ˜25 g tomato pomace was added distilled water (30 mL), acetonitrile (150 mL), BHT (2-4 mg) and GA-d₆ (2.5 μg in methanol). It was homogenized for 20 min at 9500 rpm then filtered through a sintered glass Buchner funnel. The solid residue was extracted once more with acetonitrile/water (150/30 mL), the filtrates combined and solvents evaporated, leaving a small amount of oil (˜1-2 mL). The oil was dissolved in chloroform (15 mL), dried (Na₂SO₄) and filtered, rinsing with 25 mL chloroform. Aqueous KOH (˜0.03 M, 25 mL) was added and stirred vigorously for 5 min. Layers were separated by centrifuge and the chloroform layer extracted with aqueous KOH again as above. The aqueous extracts were combined, acidified with HCl (1M, 3 mL) and extracted with chloroform (2×20 mL). The combined extracts were dried (Na₂SO₄), filtered, and solvent evaporated down to 0.3-0.5 mL. The liquid was transferred on top of a column of dry silica gel (2 cm diameter, 13.5 cm length) and N₂ passed through. After the silica was dry, the column was eluted using methanol:ethyl acetate:hexanes (5:10:85) and N₂ pressure, collecting fractions of ˜10 mL. Fractions 16-26 (co-spotting with geronic acid) were combined and solvents evaporated. The residue was esterified and the esters dissolved in 4% ethyl acetate in hexanes and filtered through cotton. Solvents were evaporated, the residue dissolved in a minimum of 4% ethyl acetate in hexanes and loaded onto a wet silica gel column (0.5 cm diameter, 33 cm length). Fractions of ˜5 mL were collected, eluting with 4% ethyl acetate in hexanes at first and switching to 15% ethyl acetate in hexanes after fraction 7. Fractions 11-17 (co-spotting with methyl geronate) were combined, solvents evaporated, and the residue dissolved in acetonitrile.

Dried Dates.

GA-d₆ (2.2 μg in methanol), acetonitrile (200 mL; 0.1% BHT) and NaCl (16 g) were added to dried dates (50-120 g) placed in a beaker containing water (HPLC grade, 120 mL). The mixture was homogenized (21,500 rpm) for ca. 8 min, giving a paste and a pale yellow solution. The solution was decanted and the paste homogenized twice more with acetonitrile and decanted as above. Rotary evaporation of the combined solutions gave a yellow oil. The oil was diluted with chloroform (50 mL) and MgSO₄ (2-3 g) was added with vigorous stirring. The resulting suspension was filtered through glass wool, followed by rinsing with chloroform (50 mL). The combined chloroform fractions were concentrated to ca. 12 mL, aqueous KOH (0.1 M; 12 mL) was added and carboxylic acids were extracted by stirring vigorously for 8 min. An emulsion was obtained that separated into two layers with centrifugation. The separated chloroform layer was extracted as before with KOH (0.1 M; 12 mL). The aqueous extracts were combined, acidified (5% aqueous HCl) to pH 2.3 and the carboxylic acids extracted with dichloromethane (2×20 mL). The dichloromethane extracts were combined, dried (MgSO₄), filtered through glass wool, the solvent evaporated and the residue esterified.

Milk.

GA-d₆ (4.5 μg in methanol), NaCl (70 g) and acetonitrile (300 mL) were added to milk (ca. 330 g) in a 1 L beaker and the mixture was homogenized for 8 min (21,500 rpm). A homogeneous white slurry formed. After standing in the refrigerator (2° C.) overnight, two layers formed. The bottom, clear layer was set aside. The top, white layer was homogenized with acetonitrile (400 mL). Separation of layers was fast, and the top clear layer collected. The clear liquid fractions were combined and concentrated by rotary evaporation, leaving an oily white suspension that was extracted with chloroform (3×7 mL), dried (Na₂SO₄) and filtered through glass wool. The filter was rinsed with chloroform (5 mL) and the combined filtrates were stirred vigorously with aqueous KOH solution (0.063 M; 25 mL) for 5 min. An emulsion formed that separated into two layers with centrifugation. The separated chloroform layer was again stirred with aqueous KOH and separated as described above. The aqueous layers were combined, acidified to pH ˜2 with 3M HCl, and extracted with chloroform (2×25 mL). The combined chloroform extracts were dried (Na₂SO₄), filtered, the solvent evaporated and the residue esterified.

Alfalfa.

Sun-cured alfalfa was ground to a powder using an electric coffee grinder. A sample (40 g) was placed in a 1 L beaker, water (120 mL) was added and the mixture left to stand for 1 hour to allow water absorption by the alfalfa to occur. Acetonitrile (550 mL) was then added, followed by GA-d₆ (140 μg in methanol) and BHT (˜0.05 g). The mixture was homogenized for 8 min (21,500 rpm) and filtered through a sintered glass Buchner funnel. The solid residue was homogenized again with acetonitrile/water (550 mL/140 mL) and filtered as described above. The combined, green filtrates were concentrated by rotary evaporation, giving a dark green oil. The oil was dissolved in chloroform (20 mL), dried with MgSO₄ and filtered through glass wool, rinsing the flask with chloroform (2×20 mL) and finally the filter with chloroform (5 mL). The combined chloroform filtrate and washings were concentrated to ˜25 mL and stirred vigorously with aqueous KOH (0.063 M; 25 mL) for 5 min. An emulsion formed, which separated into two layers with centrifugation. The separated chloroform layer was extracted again as described with aqueous KOH solution, and the aqueous layers were combined and acidified to pH ˜2 with 3M HCl. The acidic solution was extracted with chloroform (2×25 mL), dried (MgSO₄), filtered and the solvent evaporated. The residue was dissolved in 1:1 hexanes/ethyl acetate (4.5 mL) and passed through a silica gel column (2 cm×12 cm), eluting with the same solvent mixture. A colourless fraction and a green fraction were collected, followed by a yellow fraction. Solvent was evaporated from the yellow fraction and the residue esterified.

Spirulina Powder.

Distilled water (60 mL), acetonitrile (350 mL), BHT (0.06 g) and GA-d₆ (140 μg in methanol) were added to spirulina powder (13.7 g). The suspension was homogenized at 21,500 rpm for 8 min, filtered through a sintered glass Buchner funnel and the solid residue extracted again as above with acetonitrile/water (250 mL/60 mL). The filtrates were combined and the solvent removed by rotary evaporation to provide a dark green oil. A solution of the oil in chloroform (20 mL) was dried (MgSO₄) and filtered through glass wool. The flask and filter were rinsed with several portions of chloroform. All filtrates were combined and the chloroform evaporated. A dark green solid was obtained which was dissolved in 1:1 hexanes/ethyl acetate (4.5 mL) and loaded onto a column of silica gel (2.4 cm×21 cm). The column was eluted with hexanes/ethyl acetate (1.05:1). The initially colorless eluate was followed by a yellow band and then a dark green band. Approximately % of the dark green band was collected as fraction 1. The remaining dark green band and a yellow band were collected as fraction 2, until an orange band began to elute. Solvent from fraction 2 was evaporated under a stream of N₂ and the residue dissolved in chloroform (˜23 mL). The solution was stirred vigorously with aqueous KOH (0.063 M; 25 mL) for 5 min and the resulting emulsion separated by centrifugation. The aqueous layer was set aside and the chloroform layer extracted again with aqueous KOH solution (25 mL) as above. Both aqueous extracts were combined, acidified with 3M HCl to pH 1-2, and extracted with chloroform (2×25 mL). The combined chloroform extracts were dried (MgSO₄), filtered, the solvent evaporated under a stream of N₂ and the residue esterified.

Rosehip Powder.

Distilled water (30 mL), acetonitrile (150 mL), BHT (˜0.030 g) and GA-d₆ (5.7 μg in methanol) were added to rose hip powder (˜10 g) in a 400 mL beaker. The mixture was homogenized for 25 min at 9,500 rpm and then filtered through a sintered glass Buchner funnel. The filtrate was set aside and the solid residue homogenized again as described with acetonitrile/water (150 mL/30 mL). The mixture was filtered and both filtrates were combined. The solvent was evaporated and the oily residue re-dissolved in chloroform (20 mL), dried (Na₂SO₄), and filtered through glass wool. The flask and filter were rinsed with chloroform (3×10 mL) and the rinsings and filtrate combined. The solution was concentrated to ca. 25 mL and stirred vigorously for 5 min with aqueous KOH (˜0.03 M; 25 mL) solution. The suspension was separated into layers by centrifugation and the aqueous layer set aside. A second extraction of the chloroform layer with aqueous KOH was carried out as above and the aqueous layer separated by centrifugation. The aqueous layers were combined, acidified with 3 M HCl to pH 1-2 and extracted with chloroform (2×20 mL). The combined chloroform extracts were dried (Na₂SO₄), filtered through glass wool, the solvent evaporated and the residue esterified.

Fresh Cranberry.

Fresh whole cranberries (601.33 g) were placed in a beaker with acetonitrile (700 mL containing 0.01 mg/mL BHT) and GA-d₆ (0.64 μg in methanol). The mixture was homogenized at 21500 RPM for 20 min, moving the homogenizer around carefully to break open all the berries. The resulting pulp was blended 25 min more, then filtered through a coarse sintered glass Buchner funnel. The solid residue was homogenized again for 25 min with acetonitrile (700 mL) and water (50 mL). It was filtered again as above and the solvents evaporated to give purple-red oil. Chloroform (100 mL) and water (10 mL) were added and the mixture stirred at 40° C. to dissolve. Na₂SO₄ (ca. 250 g) was added, mixed, and the mixture was filtered through cotton, rinsing with chloroform (50 mL). The filtrate was stirred vigorously with aqueous KOH (50 mL of ˜0.03 M) for 5 min then transferred to a separatory funnel. Most of the chloroform was separated and the remaining liquid was centrifuged. The aqueous layer was set aside and the chloroform layer extracted once more as above. Aqueous layers were combined, acidified with aqueous HCl (1 M, ˜6 mL), and extracted with chloroform (2×50 mL), centrifuging as necessary to separate the layers. The combined chloroform extracts were dried (Na₂SO₄), filtered, concentrated to ca. 3 mL and transferred to the top of a column of dry silica gel (13.5 cm long×2 cm diameter). N₂ was forced through to dry the silica, and the column was eluted using N₂ pressure and a solvent system of 5:10:85 methanol:ethyl acetate:hexanes, collecting fractions of ca. 10 mL. Fractions 19-35 (co-spotting with GA) were combined, solvents were evaporated and the residue esterified.

Cranberry Powder.

Distilled water (30 mL), acetonitrile (150 mL), BHT (˜0.015 g) and GA-d₆ (0.75 μg in methanol) were added to cranberry powder (˜4.8 g) in a 400 mL beaker. The mixture was homogenized for 25 min at 9,500 rpm and then filtered through a sintered glass Buchner funnel. The filtrate was set aside and the solid residue homogenized again as described with acetonitrile/water (150 mL/30 mL). The mixture was filtered and both filtrates combined. The solvent was evaporated and the residue re-dissolved in chloroform (20 mL), dried (Na₂SO₄), and filtered through glass wool. The flask and filter were rinsed with chloroform (4×5 mL) and the rinsings and filtrate combined. The solution was concentrated to ca. 25 mL and stirred vigorously for 5 min with aqueous KOH (˜0.03 M; 25 mL). Layers were separated by centrifugation and the aqueous layer set aside. A second extraction of the chloroform layer with aqueous KOH solution was carried out as above and the aqueous layer separated by centrifugation. The aqueous layers were combined, acidified with 3 M HCl to pH ˜1 and extracted with chloroform (2×20 mL). The combined chloroform extracts were dried (Na₂SO₄), filtered through cotton and the solvent evaporated. The residue was dissolved in ˜1 mL methanol (note: some fine solid remains undissolved) and the liquid transferred as evenly as possible to the top of a column of dry silica gel (2 cm×10 cm), rinsing the column walls with methanol (0.2 mL). N₂ was forced through the column to dry the silica, and the column then eluted at ca. 5 mL/min with an 85/10/5 hexanes/ethyl acetate/methanol solution. Fractions of 5 mL were collected and those co-spotting with geronic acid were combined, the solvent evaporated and the residue esterified.

Paprika Powder.

Distilled water (30 mL), acetonitrile (150 mL), BHT (3-5 mg) and GA-d₆ (4.0 μg in methanol) were added to paprika powder (˜5.0 g) in a 400 mL beaker. The mixture was homogenized for 25 min at 9,500 rpm and then filtered through a sintered glass Buchner funnel. The filtrate was set aside and the solid residue homogenized again as described with acetonitrile/water (150 mL/30 mL). The mixture was filtered and both filtrates combined. The solvent was evaporated and the oily residue re-dissolved in chloroform (15 mL), dried (Na₂SO₄), and filtered through cotton. The flask and filter were rinsed with chloroform (4×5 mL) and the rinsings and filtrate combined. The solution was stirred vigorously for 5 min with aqueous KOH (˜0.03 M; 25 mL) and the mixture separated into two layers by centrifugation. The separated chloroform layer was extracted as above with aqueous KOH (25 mL). The aqueous layers were combined, acidified with 1 M HCl to pH 1-2 and extracted with chloroform (2×25 mL). The combined chloroform extracts were dried (Na₂SO₄), filtered through cotton, the solvent evaporated and the residue esterified.

Sweet Potato Powders #1 and #2.

Distilled water (25 mL), acetonitrile (120 mL), BHT (4-6 mg) and GA-d₆ (4.0 μg in methanol) were added to sweet potato powder (5-8 g). The mixture was homogenized at 13,500 rpm for 25 min, then filtered through a sintered glass Buchner funnel. The solid was extracted again as described with acetonitrile/water (120/25 mL) and the filtrates were combined. Solvents were removed on the rotary evaporator until a small amount of oil remained (˜1-2 mL). Chloroform (12 mL) was added to dissolve the oil and the mixture was dried with Na₂SO₄. It was filtered through cotton, rinsing with chloroform (21 mL). Aqueous KOH (˜0.03 M, 25 mL) was added to the combined filtrates and stirred vigorously for 5 min. The aqueous layer was separated and the chloroform extracted again with aqueous KOH as above. The combined aqueous extracts were acidified with aqueous HCl (1 M, ˜3 mL) then extracted with chloroform (2×20 mL). The combined chloroform extracts were dried (Na₂SO₄), filtered, solvents evaporated and the residue esterified.

Seaweeds (Dulse Powder, Nori Flakes).

To ca. 5.0 g seaweed was added distilled water (25 mL), acetonitrile (120 mL), BHT (2-4 mg), and GA-d₆ (21 μg in methanol). It was homogenized at 9500 RPM for 20 min then filtered through a sintered glass Buchner funnel. The solid residue was extracted again as above with acetonitrile/water (120/25 mL), the filtrates combined, and concentrated on the rotary evaporator to leave ˜1-2 mL oil. Chloroform (18 mL) was added to dissolve the mixture, which was dried (Na₂SO₄) and filtered through cotton, rinsing with chloroform (˜22 mL). It was stirred vigorously with aqueous KOH (˜0.03 M, 25 mL) for 5 min then separated by centrifuge. The aqueous layer was collected and the chloroform layer extracted with aqueous KOH again as above. The combined aqueous extracts were acidified (1 M HCl, ˜3 mL) and extracted with chloroform (2×20 mL). The combined chloroform extracts were dried (Na₂SO₄), filtered, solvents evaporated and the residue esterified.

Wheatgrass Powder.

To ca. 5.5 g wheatgrass powder was added GA-d₆ (10 μg in methanol), acetonitrile (120 mL), water (30 mL) and BHT (˜3 mg). The mixture was homogenized at 6,500 rpm for 15 min, then filtered through a coarse sintered glass Buchner funnel. The solid residue was extracted once more as above with acetonitrile/water (120 mL/30 mL), the filtrates were combined and solvents evaporated until ˜1-2 mL green oil remained. Chloroform (15 mL) was added to dissolve the oil, which was dried (Na₂SO₄) and filtered, rinsing with chloroform (25 mL). The combined filtrates were stirred vigorously with aqueous KOH (25 mL; ˜0.03 M) for 5 min and the mixture was transferred to a separatory funnel. Most of the chloroform was separated and the remaining liquid was centrifuged. The chloroform layer was extracted again with aqueous KOH as above, and the aqueous layers were combined, acidified with aqueous HCl (1 M, ˜3 mL) and extracted with chloroform (2×20 mL). The combined extracts were dried (Na₂SO₄), filtered, solvents removed on the rotary evaporator and the residue was esterified.

Red Palm Oil.

To red palm oil (ca. 28.0 g) was added BHT (2-3 mg), hexanes (20 mL) and geronic acid-d₆ (2.0 μg in methanol). It was stirred 10 min then acetonitrile (20 mL) was added and stirred vigorously 5 min. Layers were separated and the acetonitrile layer (top) was collected. It was stirred vigorously with hexanes (20 mL) for 5 min, then the acetonitrile layer (bottom) was collected and solvent evaporated. Aqueous NH₃ (5%, 6 mL) and distilled water (3 mL) was added to the residue and stirred vigorously to obtain a cloudy orange liquid. An SPE cartridge (Waters Oasis MAX, 6 cc/500 mg) was prepared by passing through sequentially methanol (6 mL), distilled water (6 mL) and aqueous NH₃ (0.5%, 4.5 mL). The orange liquid was passed through the cartridge, which was eluted sequentially with aqueous NH₃ (0.5%, 4.5 mL), methanol (9 mL) and acidic methanol (2% HCl, 4.5 mL). The acidic methanol fraction was collected, solid NaHCO₃ added and stirred until bubbling ceased, and the mixture esterified.

Milk Powder.

To ca. 105 g whole milk powder was added GA-d₆ (0.5 μg in methanol), ethyl acetate (280 mL) and BHT (˜3 mg). The mixture was stirred vigorously for 20 min then filtered through a medium sintered glass Buchner funnel. The solid residue was extracted once more with ethyl acetate (280 mL) as above, and the combined filtrates were concentrated on the rotary evaporator. The residue was dissolved in chloroform (15 mL), dried (Na₂SO₄) and filtered, rinsing with chloroform (30 mL). The combined filtrates were stirred vigorously with aqueous KOH (25 mL; ˜0.03 M) for 5 min and layers were separated. The chloroform layer was extracted once more with aqueous KOH as above, and the combined aqueous extracts were acidified with aqueous HCl (1 M, ˜3 mL) and extracted with chloroform (2×20 mL). The combined extracts were dried (Na₂SO₄), filtered, solvent was evaporated, and the residue esterified.

Whole Egg Powder.

To ca. 25 g whole egg powder was added GA-d₆ (1-2 μg in methanol), acetonitrile (120 mL), water (30 mL) and BHT (˜3 mg). The mixture was homogenized at 13,500 RPM for 10 min, then filtered through a coarse sintered glass Buchner funnel. The solid residue was extracted once more with acetonitrile/water (120 mL/30 mL), the filtrates were combined and solvents evaporated by blowing a stream of N₂ overnight (use of the rotary evaporator was hindered due to excess foaming). Chloroform (15 mL) was added to dissolve the residue, which was dried (Na₂SO₄) and filtered, rinsing with chloroform (135 mL). The combined filtrates were stirred vigorously with aqueous KOH (50 mL; ˜0.03 M) for 5 min then NaCl (5 g) was added, stirred 1 min and the mixture transferred to a separatory funnel. Most of the chloroform layer separated and the remaining liquid was centrifuged. The chloroform layer was extracted once more as above with aqueous KOH. Aqueous extracts were combined, acidified with aqueous HCl (1 M, ˜6 mL) and extracted with chloroform (2×25 mL). The combined extracts were dried (Na₂SO₄), filtered, solvents removed on the rotary evaporator and the residue was esterified.

### Geronic Acid Analysis

GC-MS Analysis.

A GC-MS-based assay was employed using hexadeuterated GA, GA-d₆, as an internal standard.¹³ Calibrations were carried out prior to analysis of each food sample. Stock solutions of GA and GA-d₆, prepared in methanol in strengths related to anticipated sample levels (1.5-38 μg/mL), were combined in a range of ratios (1:4 to 4:1) to provide calibration samples. After the solutions were combined (1.0-1.5 mL total volume) in 20 mL scintillation vials, they were diluted to 4.5 mL with methanol and esterified with trimethyloxonium tetrafluoroborate following the procedure described below. Esterified samples obtained after solvent removal under a stream of nitrogen or by rotary evaporation were dissolved in acetonitrile for analysis by GC-MS. Comparison of the abundance of ions m/z=154 and 160, for GA and GA-d₆, respectively, in SIM mode was used for calibration and quantitation of GA. Retention times of GA and GA-d₆ methyl esters were determined with reference standards. Calibration curves were constructed by plotting the ratio of the m/z=154 and 160 ion intensities, I/I₆, versus the corresponding mass ratio of the GA and GA-d₆ standards, m/m₆. The data were fitted by least squares analysis to equation (1), where a is the slope and b is the γ-intercept.

I/I₆=a(m/m₆)+b (1)

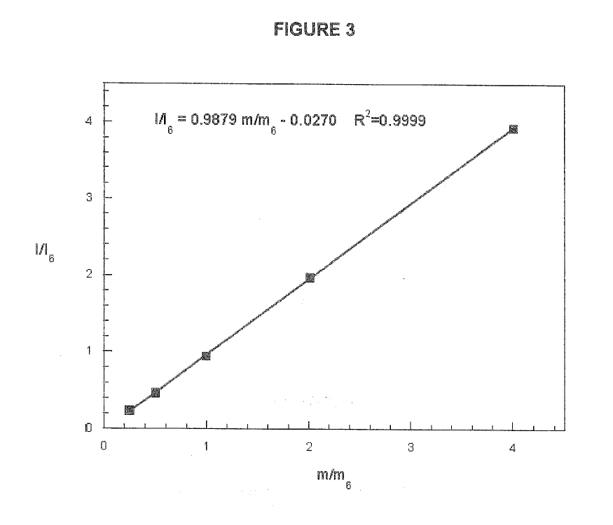

The amount of GA, m, in a food sample was calculated from the I/I₆ value of the sample obtained for addition of a known amount of GA-d₆, m₆, using equation 1 and the values of a and b obtained from the calibration curve. An example of a typical calibration curve is provided in FIG. 3.

The identity of endogenous GA and added GA-d₆ methyl esters in food samples was confirmed by their GC retention times and mass spectra compared to prepared standards. The mass spectrum of GA methyl ester gave a match of 90-93% with the GC-MS library. The GA methyl ester shows intense ions at m/z=154, 129 and 102. These ions correspond, respectively, to the [M⁺-methanol], [M⁺-MeC(O)CH₂], [M⁺-MeC(O)CH₂CHCH₂] fragments of the parent ion. The relative intensity of the parent molecular ion (m/z=186; 2%) was too low for analysis. Although ions 129 (40%) and 102 (100%) have high intensities, they were found to be subject to interference by ions generated from other compounds in the extracted food samples. Ion 154 (ca. 20%), however, was rarely found to be subject to such interference, so it was selected for monitoring for measurement of the GA methyl ester. Similarly, ion 160 was used for the GA-d₆ methyl ester.

GC-MS calibration curves of GA vs. GA-d₆, prepared for each food sample by plotting the ratio of intensities of the 154 vs. 160 ions versus the ratio of associated sample concentrations, gave excellent linear responses, as illustrated in FIG. 3.

Carrot Juice.

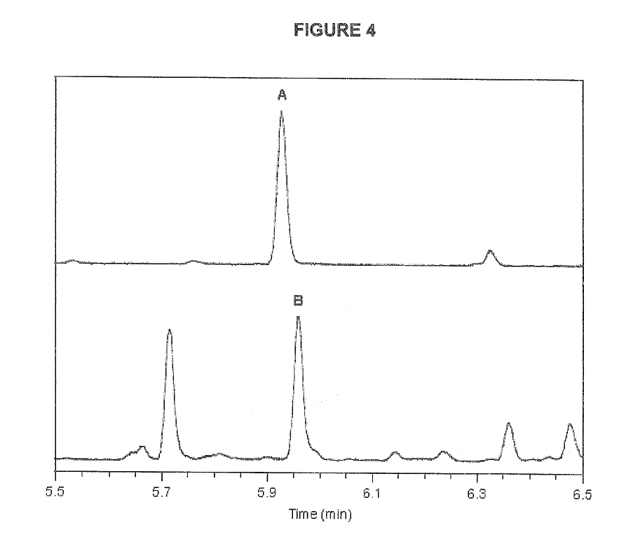

The GC-MS chromatogram of a carrot juice analyte shows clearly distinguishable signals of the GA and GA-d₆ methyl esters. The retention times of the esters were confirmed by comparison to those of pure standard compounds and the library match to geronic acid methyl ester was 90%. The GC-MS chromatogram of the analytes recorded in SIM mode showed clear, separated signals of the GA and GA-d₆ methyl esters (FIG. 4). The signals were integrated and the ratio of intensities of GA and GA-d₆ methyl esters (I₀/I₆) was used to calculate the concentration of GA in the carrot juice using values for the parameters of equation (1) obtained from a calibration curve (e.g., FIG. 3). The average recovery of added GA-d₆ was 89% (77-102% range), based on the GA-d₆ signal intensity in the chromatogram compared with the intensity of a standard solution measured separately.

Raw Tomato.

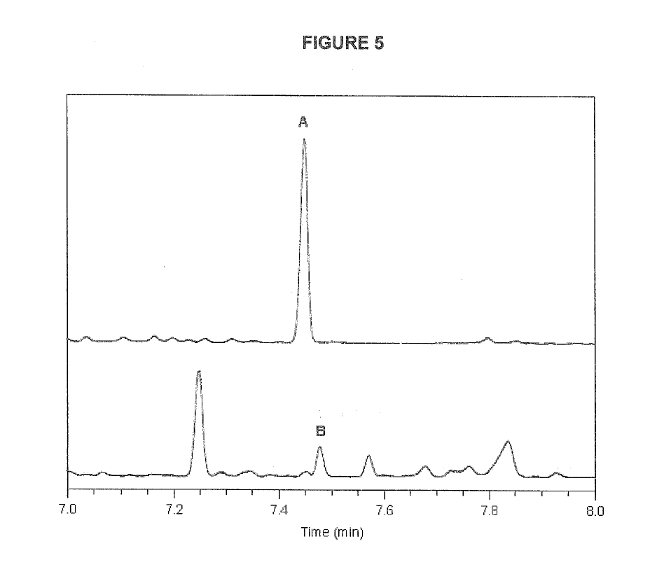

The signals of GA and GA-d₆ methyl esters were clearly visible in the GC-MS chromatogram for raw tomatoes. The library match to the GA methyl ester was 93%. The GC-MS chromatogram of the analytes recorded in SIM mode showed clear, separated signals of the GA and GA-d₆ methyl esters (FIG. 5). The mean concentration of GA (1.5±0.9 ng/g; (Table 1B) was approximately ten times lower than for carrot juice (12.6±0.8 ng/g; Table 1A, in line with the substantially lower level of β-carotene present in tomatoes (Table 3). The wider variation in GA values for tomato is attributed to the low level of GA and the sensitivity limitations of the analytical method.

### Results

### Analysis of GA in Food Samples

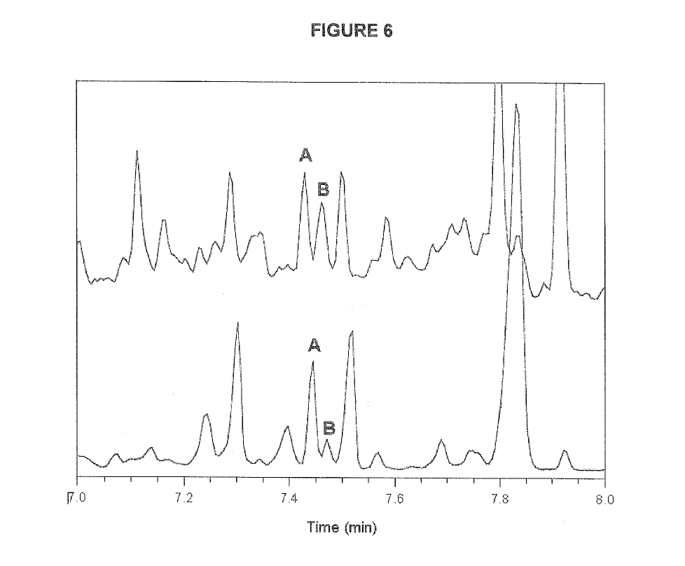

FIG. 6 illustrates GC-MS chromatograms of analytes of carrot juice and raw tomato showing clearly distinguishable signals for the GA and GA-d₆ methyl esters. The identities of the esters were confirmed by comparison of their retention times with those of the pure standard compounds together with a mass spectral library match to geronic acid methyl ester.¹⁹

Taking the example of carrot juice, the GC-MS chromatogram of the analyte recorded in SIM mode showed clear, separated signals of the GA and GA-d₆ methyl esters (FIG. 4). The signals were integrated and the ratio of intensities of GA and GA-d₆ methyl esters (I₀/I₆) was used to calculate the concentration of GA in the carrot juice using values for the parameters of equation 1 obtained from a calibration curve.

Further confirmation of the presence of GA was obtained by purification through semicarbazone derivatization. Table 1A shows that semicarbazone purification gave values closely similar to those of the direct method for both samples 1 and 2.

Table 1A also illustrates the need for antioxidant protection to minimize adventitious oxidation during sample processing. In the absence of added antioxidant, sample 1, processed for 32 h, had a higher GA value than did sample 2, which was processed for 8 h. Addition of ca. 0.1% BHT to the extraction solvent results in GA values that are markedly and consistently lower (Table 1A, samples 3-5). Also, the GA value obtained via semicarbazone derivatization in the presence of BHT (Table 1, sample 6) was similar to the values obtained directly for samples 3-5.

Analysis of a variety of food samples shows GA values vary over a wide range (Table 2). The much higher values seen for dried, provitamin A-rich foods, particularly those analyzed in powdered form (e.g., carrot, spirulina, seaweed, alfalfa and wheatgrass), confirm that exposure of these foods to air, heat and light during drying causes substantial and varying degrees of adventitious β-carotene oxidation. The highest value is observed for carrot powder #1 at 840 times the value for carrot juice, corresponding to an almost 100-fold enrichment in GA when compared on a dry weight basis. Of note, this powder as received was a pale brown color, indicating a very low level of β-carotene, which was confirmed by a UV measurement that showed β-carotene to be below the limit of detectability. Apparently all of the β-carotene present had been oxidized.

A second commercial carrot powder (#2) was orange-colored, containing ˜120 μg/g β-carotene. Accordingly, the level of GA is substantially lower, at approximately half the value for carrot powder #1.

For raw tomatoes the concentration of GA is approximately ten times lower than in carrot juice, in line with a considerably lower level of β-carotene in tomatoes. The level in raw cranberry also is low, consistent with the low levels of provitamin A carotenoids in this fruit.

Dried spirulina, seaweed, alfalfa and wheatgrass show high levels of GA. Alfalfa is an important source of carotenoids in animal feed and is used in the production of bovine milk in North America. Accordingly, samples of milk and milk powder (3.25% milk fat each) were analyzed and found to contain a small amount of GA. Whole egg powder, another animal-derived product, contained more GA than the milk products.

Red palm oil, a rich source of α- and β-carotenes that are naturally protected against oxidation by the presence of vitamin E, nevertheless contains a modest amount of GA.

No GA was detected in echinacea purpurea root powder, honey or bee pollen, none of which are known sources of β-carotene. Nor were detectable amounts of GA found in yellow corn flour or brown rice flour.

### Estimation of Provitamin A Carotenoid-Oxygen Copolymer Content.

Knowing the extent of GA formation relative to polymer formation in the inventors' earlier model β-carotene oxidation study, approximate estimates were made of the levels of the predominantly polymeric total β-carotene oxidation product mixture. In the oxidation of β-carotene GA forms at a rate of roughly 2% of the level of the total product, OxBC.¹³ As a first approximation in estimating total oxidation product levels in food, it was assumed that all GA comes from β-carotene. However, the β-ionone ring structure capable of conversion into GA is present in the other provitamin A carotenoids (FIG. 2). β-carotene with two rings can form two GA per molecule, whereas α-carotene, γ-carotene and β-cryptoxanthin with one ring can form just one GA per molecule. The provitamin A carotenoids (PVA) were not measured in the food samples analyzed in this study. Instead literature sources were used to obtain approximate nominal values for comparison with the total estimated level of oxidized provitamin A carotenoids (Table 2). Given that in a few samples there will be some contributions from one or two of the minor provitamin A carotenoids (e.g., α-carotene in carrots), the estimated total oxidation product is designated by the term OxPVA, representing the sum of the contributions from each carotenoid. Note that lycopene, the major carotenoid in tomatoes, lacking any ring structure, was confirmed not to form GA when oxidized.

Values of OxPVA calculated from GA values for each food are shown in Table 2. A comparison of the OxPVA value for each food to the corresponding estimated level of total provitamin A carotenoids, PVA, originally present in the raw food and adjusting for water content as appropriate, provides a rough estimate of carotenoid loss by oxidation, expressed as a percentage of PVA, i.e., OxPVA/PVA (column 6, Table 2).

The OxPVA/PVA data show carrot juice and raw tomatoes have low levels of oxidized β-carotene at ˜1%. In striking contrast, dried foods show moderate to high percentage levels of oxidized products. The upper value for full conversion of PVA to OxPVA would be around 130% (OxBC is ca. 1.3 times heavier than β-carotene). Carrot powder #1 shows the highest value, corresponding to an apparent 55% conversion of the nominal level of original carotenes, although, as already noted, the actual level of β-carotene in this product was undetectable. Therefore, the actual OxPVA/PVA value should be close to 130%, corresponding to complete oxidative conversion, which suggests the assumed OxPVA/GA ratio should be more than 50.

Spirulina powder, nori seaweed flakes, dulse seaweed powder, sun-cured alfalfa, wheatgrass powder and sweet potato powder also are relatively significant sources of oxidation products. Spirulina powder, with a very high level of residual β-carotene, nevertheless is notable as an OxPVA source, even at only an apparent 1% oxidative conversion of the nominal original β-carotene level.

For the most part, the OxPVA/PVA values for the plant-based products lie within the 130% limit. The exceptions are cranberry powder and dried dates. We attribute uncertainty in the actual level of β-carotene in the raw fruit, possibly compounded by the low level of β-carotene in cranberries also affecting the accuracy of the GA determination, as factors affecting the accuracy of these estimates.

The projected OxPVA/PVA values in milk and whole egg powder also exceed 130% by large margins. GA may not be predictive of OxPVA in animal products and could be influenced by dietary sources, e.g., alfalfa, and possibly by oxidation of endogenous vitamin A.

### Example 3—Direct Isolation of a Carotenoid-Oxygen Copolymer Compounds

### Procedure for Isolating Carotenoid-Oxygen Copolymers.

In general, ethyl acetate containing BHT (0.05 mg/mL) was mixed with the food powder and the mixture allowed to sit overnight. The next day, the slurry was filtered through a sintered glass Buchner funnel, rinsing the residue with ethyl acetate containing BHT (0.05 mg/mL). Filtrates were combined and concentrated on a rotary evaporator, filtered again, and the solvent evaporated. A minimum of polar solvent (ethyl acetate or ethyl acetate/methanol) was used to dissolve the residue, followed by precipitation through careful addition of hexanes. The supernatant was decanted, the residue rinsed with hexanes, and then dissolved in ethyl acetate or ethyl acetate/methanol. The solution was filtered as necessary and the precipitation process repeated up to two more times. The final product was then dried under vacuum.

Detailed descriptions of extractions for dried forms of carrot, tomato, rosehip, paprika, dulse seaweed, alfalfa, wheatgrass and tomato pomace are provided below.

Carrot Powder #1.

The powder (80 g) was placed in a flask, mixed with ethyl acetate (120 mL), stirred for 7 h and allowed to sit for 3 days. The mixture was filtered through sintered glass, rinsing with ethyl acetate (2×90 mL). The solvent was removed on a rotary evaporator, the residue dissolved in ethyl acetate (2 mL) and allowed to sit for 30 min while white material precipitated. The liquid was filtered through a 0.2 μm syringe filter (rinsing with ethyl acetate) and solvent evaporated to give a caramel-colored oil (898 mg). The oil was dissolved in ethyl acetate (1.2 mL) and hexanes (50 mL) was added dropwise with stirring. After complete addition, it was stirred 30 min, the liquid was decanted and the residue rinsed with hexanes (2×3 mL). The residue was dissolved in ethyl acetate, then the solvent was removed on the rotary evaporator and dried on the vacuum pump for 1 h to give 78 mg brown solid.

The solid was combined with another sample prepared in a similar manner for a total of 218 mg. The solids were dissolved in ethyl acetate (1 mL), filtered through a 0.2 m syringe filter, and hexanes (50 mL) was added dropwise with stirring. After 1 h, the liquid was decanted and the precipitate rinsed with hexanes (3×1.5 mL). The solid was dissolved in ethyl acetate, then the solvent removed on the rotary evaporator and residue dried on the vacuum pump for 2 h to give 195 mg brown solid.

The solid (155 mg) was precipitated a third time from ethyl acetate (0.5 mL) and hexanes (5 mL), rinsing with hexanes (3×1.5 mL). Drying on the vacuum pump for 3 h gave 139 mg brown solid.

Carrot Powder #2.

The powder (502 g) was covered with ethyl acetate (ca. 450 mL, 0.05 mg/mL BHT) and allowed to sit overnight (17 h). It was filtered through a sintered glass Buchner funnel in 3 separate portions, rinsing each with ethyl acetate (2×90 mL, 0.05 mg/mL BHT). The filtrates were combined and concentrated on the rotary evaporator, leaving ˜14 mL of solution. It was filtered through a 0.2 m syringe filter, rinsing with ethyl acetate (3×3 mL, 0.05 mg/mL BHT). Evaporation of the solvent on the rotary evaporator gave 4.7292 g dark red oil. The oil was diluted with ethyl acetate (2 mL) and hexane (100 mL) was added dropwise with stirring. After 30 min, the liquid was decanted and the residue rinsed with hexanes (3×3 mL). The solid was dissolved in ethyl acetate, then the solvent removed on the rotary evaporator and the product dried on the vacuum pump to give 269 mg viscous, reddish-orange oil.

The oil was dissolved in ethyl acetate (0.5 mL) and hexanes (10 mL) was added dropwise with stirring. After 30 min, the liquid was decanted and the residue rinsed with hexanes (3×1.5 mL). The residue was dissolved in ethyl acetate, then the solvent was evaporated and product dried on the vacuum pump for 45 min to give 215 mg solid.

The solid was precipitated once more from ethyl acetate (0.5 mL) and hexanes (5 mL), rinsing the residue with hexanes (3×1.5 mL). Drying the product on the vacuum pump for 2 h gave 203 mg orange solid.

Tomato Powder.

The powder (154 g) was covered with ethyl acetate (320 mL, 0.05 mg/mL BHT) and allowed to sit overnight (17 h). The mixture was filtered through a sintered glass Buchner funnel, rinsing with ethyl acetate (2×100 mL; 0.05 mg/mL BHT). The filtrates were combined and concentrated, leaving ˜14 mL of solution. It was filtered through cotton (rinsing 3×3 mL ethyl acetate), concentrated to ˜7 mL and filtered through a 0.2 μm syringe filter (rinsing with small portions of ethyl acetate totaling 3 mL). Evaporation of the solvent gave 2.15 g red oil. The oil was dissolved in ethyl acetate (2.5 mL) and hexanes (100 mL) was added with stirring. After 30 min, the liquid was decanted and solid precipitate rinsed with hexanes (3×3 mL). The precipitate was dissolved in ethyl acetate (15 mL) and a small amount of insoluble white material was removed by suction filtration through filter paper (rinsing with 10 mL ethyl acetate). Evaporation of the solvents on the rotary evaporator followed by drying on the vacuum pump for 1 h gave red solid (453 mg).

The red solid was dissolved in ethyl acetate (5 mL). A small amount of white precipitate would not dissolve, which was removed by centrifugation. The solvent was evaporated and residue re-dissolved in ethyl acetate (1.3 mL). Hexane (13 mL) was added dropwise with stirring, and after 30 min the liquid was decanted, rinsing the precipitate with hexanes (3×1.5 mL). The precipitate was dissolved in ethyl acetate (slow process, took 1 h warming on a rotary evaporator bath at 35-50° C.), then the solvent removed on the rotary evaporator and dried on the vacuum pump for 2.5 h to give a red solid (400 mg).

Tomato Pomace.

Tomato pomace (505 g) was covered with ethyl acetate (˜1.5 L; 0.05 mg/mL BHT) and allowed to sit overnight. It was filtered through a sintered glass Buchner funnel in four portions, rinsing each with ethyl acetate (3×80 mL, 0.05 mg/mL BHT). The combined solvents were removed on the rotary evaporator and the residual oil was dried under a stream of N₂ for 2 h to give 65.28 g red oil. Hexane (500 mL) was added to the oil dropwise with stirring and the mixture allowed to stir overnight. In the morning the liquid was decanted and the residue rinsed with hexanes (5×4.5 mL). The precipitate was dissolved in ethyl acetate (9 mL) and filtered through a 0.45 m syringe filter, rinsing with ethyl acetate (4×3 mL). Solvents were removed on the rotary evaporator and the residue dried on the vacuum pump to give 832 mg thick, resin-like red oil.

The oil was dissolved in ethyl acetate (2 mL) and precipitated with hexanes (40 mL), allowing the mixture to stir 30 min. The liquid was decanted and the residue rinsed with hexanes (4×1.5 mL) and dissolved in ethyl acetate. Solvents were evaporated and the residue dried on the vacuum pump to give 690 mg red resin. Another precipitation was carried out using ethyl acetate (1.5 mL) and hexanes (15 mL), rinsing the precipitate with hexanes (4×1.5 mL). Evaporation of solvents and drying the product on the vacuum pump for 3 h gave 632 mg red resin.

The resin was dissolved in ethyl acetate (1 mL) and filtered through a 0.2 μm syringe filter, rinsing with small portions of ethyl acetate totaling 1 mL. Hexane (20 mL) was added dropwise to the combined filtrate to give a precipitate. The liquid was decanted and the residue rinsed with hexanes (4×1.5 mL). Drying the residue on the vacuum pump gave 509 mg red solid.

Alfalfa.

Sun-cured alfalfa was milled in a coffee grinder for ˜20 sec to give coarsely ground material (263 g). It was covered with ethyl acetate (0.05 mg/mL BHT) and allowed to sit overnight. The next day, it was filtered through a sintered glass Buchner funnel in four separate portions, rinsing each with ethyl acetate (2×150 mL; 0.05 mg/mL BHT). The filtrates were combined, concentrated on the rotary evaporator to ca. 55 mL, and filtered through a 0.45 μm syringe filter, rinsing with ethyl acetate (3×3 mL; 0.05 mg/mL BHT). Evaporation of the solvent gave a thick, dark green gel (3.68 g).

Ethyl acetate (6.5 mL) was added to the gel, which did not dissolve it completely. It was filtered again through a 0.45 m syringe filter, rinsing with ethyl acetate (4×1 mL). Solvents were evaporated to give a thick, dark green oil (3.39 g).

The oil was dissolved in ethyl acetate (6 mL) and hexanes (250 mL) was added dropwise with stirring. After complete addition, stirring was stopped and the mixture was allowed to sit overnight. In the morning, the liquid was decanted and the residue rinsed with hexanes (4×3 mL). The solid was dissolved in ethyl acetate, then solvents removed on the rotary evaporator to give 345.4 mg thick, dark green oil.

The oil was dissolved in ethyl acetate (2 mL) and hexane (50 mL) was added dropwise with stirring. After 30 min, the liquid was decanted and the residue rinsed with hexane (4×1.5 mL). The solid was dissolved in ethyl acetate (3 mL) and filtered through a 0.2 m syringe filter, rinsing with ethyl acetate (4×0.7 mL). Evaporation of the solvent on the rotary evaporator followed by drying on the vacuum pump for 30 min gave a dark green solid (292 mg).

The solid was dissolved in ethyl acetate (1 mL) and hexane (10 mL) was added dropwise with stirring. After 30 min, the liquid was decanted and the solid residue rinsed with hexanes (3×1.5 mL). The residue was dissolved in ethyl acetate (2.5 mL) and filtered through a 0.2 μm syringe filter (rinsing 4×0.5 mL ethyl acetate) The combined solvents were evaporated and the residue dried on the vacuum pump for 3 h to give a dark green solid (257 mg).

Rosehip Powder.

The powder (405 g) was covered with ethyl acetate (400 mL, 0.05 mg/mL BHT) and allowed to sit overnight. In the morning it was filtered in two portions through a sintered glass Buchner funnel, rinsing each with ethyl acetate (2×100 mL, 0.05 mg/mL BHT). Solvents were removed on the rotary evaporator and ethyl acetate (40 mL) was added to the residue. After 1.5 h of stirring some white precipitate was observed. It was removed by centrifuge, rinsing the tubes with ethyl acetate (ca. 8 mL total). The liquid fractions were combined and concentrated on the rotary evaporator to ca. 15 mL. More precipitate was observed, which was removed by centrifugation as above. The liquid fractions were combined and solvents evaporated to give an orange solid (2.53 g). Ethyl acetate (15 mL) was added and the mixture stirred overnight. Some fine white precipitate was visible in the morning. Hexane (200 mL) was added dropwise with stirring, and after 30 min, the cloudy mixture was suction filtered through paper, rinsing with hexanes (4×3 mL). The residue was dissolved in ethyl acetate:methanol (1:1, 4 mL) at 40° C., then the solvents were removed on the rotary evaporator and residue dried overnight on the vacuum pump to give 560 mg orange solid.

Paprika.

Paprika (232 g) was covered with ethyl acetate (ca. 300 mL; 0.05 mg/mL BHT) and allowed to sit overnight. The mixture was filtered through a sintered glass Buchner funnel, rinsing with ethyl acetate (3×80 mL, 0.05 mg/mL BHT). The filtrates were combined and concentrated on the rotary evaporator to ca. 30 mL. It was filtered through a 0.45 μm syringe filter (rinse 3×3 mL ethyl acetate), solvents were removed on the rotary evaporator and the residue dried under a stream of N₂ for 4.5 h to give dark red oil (25.7811 g). Ethyl acetate (1 mL) was added to the oil, followed by dropwise addition of hexanes (65 mL) with stirring. After 1 h the liquid was decanted and the residue rinsed with hexanes (4×3 mL). Ethyl acetate (10 mL) was added to the residue but did not completely dissolve it; addition of methanol (3 mL) allowed it to dissolve. Solvents were removed on the rotary evaporator and the residue dried on the vacuum pump to give thick red oil (306 mg).

The oil was dissolved in ethyl acetate:methanol (2:1, 0.5 mL) and hexanes (25 mL) was added dropwise with stirring. After 30 min, a thick red oil coated the bottom and sides of the flask, separate from a clear orange liquid layer on top. The orange liquid was removed by pipette and the red oil rinsed twice with hexanes (6 mL+3 mL). The red oil was dissolved in ethyl acetate:methanol (2:1), solvents were removed on the rotary evaporator and the residue dried on the vacuum pump. As the solvent evaporated, the oil became thicker, making it more difficult to dry. Further drying was achieved by using a stream of N₂ to disturb the surface of the resin and then placing it on the vacuum pump with gentle heating for ca. 5 min until bubbling ceased. After several cycles alternating between an N₂ stream and vacuum pumping, the resin was placed in a 45-50° C. oil bath and dried under vacuum for 1 h, giving a viscous, dark red oil (250 mg).

Dulse Seaweed Powder.

The powder (351 g) was covered with ethyl acetate (ca. 400 mL; 0.05 mg/mL BHT) and allowed to sit overnight. In the morning the mixture was filtered through a sintered glass Buchner funnel in two portions, rinsing each with ethyl acetate (2×100 mL; 0.05 mg/mL BHT). The filtrates were combined and concentrated on the rotary evaporator down to ca. 9 mL. After sitting for 1 h, the liquid was filtered through a 0.45 μm syringe filter, rinsing with ethyl acetate (3×1.5 mL). The filtrates were combined, solvents were removed on the rotary evaporator, and the residue dried under a stream of N₂ for 20 min to give thick, dark green oil (1.79 g).

The oil was dissolved in ethyl acetate (1 mL) and hexanes (50 mL) was added dropwise with stirring. After 1 h, the liquid was decanted and residue rinsed with hexanes (4×1.5 mL). The residue was dissolved in ethyl acetate, then solvents were evaporated and the residue dried on the vacuum pump for 45 min to give dark green, viscous oil (339 mg).

The oil was dissolved in ethyl acetate (1 mL) and hexane (25 mL) was added dropwise with stirring. After 30 min, the liquid was decanted and the residue rinsed with hexane (3×1.5 mL). The residue was dissolved in ethyl acetate, then solvents were evaporated and the product dried on the vacuum pump for 1 h 45 min to give a dark green solid (264 mg).

A third precipitation was carried out as before using ethyl acetate (1 mL) and hexane (10 mL), rinsing with hexanes (3×1.5 mL). The solid was dissolved in ethyl acetate and filtered through a 0.2 μm syringe filter. Solvents were evaporated under a stream of N₂, and the residue dried on the vacuum pump for 1 h 40 min to give a dark green solid (222 mg).

Wheatgrass Powder.

Wheatgrass powder (401.09 g) was mixed with ethyl acetate (700 mL containing 0.05 mg/mL BHT) in a 1 L beaker, covered with aluminum foil and allowed to sit for three days. The slurry was filtered through a coarse sintered glass Buchner funnel in two portions, rinsing each portion with ethyl acetate (3×70 mL, containing 0.05 mg/mL BHT). The filtrates were combined and solvents removed on the rotary evaporator to give a dark green, highly viscous liquid. It was dissolved in ethyl acetate (10 mL) and hexanes (500 mL) were added dropwise with stirring. One hour after complete addition, the liquid was suction filtered through paper, rinsing with hexanes (4×3 mL). The residue was dissolved in ethyl acetate (30 mL) and filtered through a 0.45 Gm syringe filter, rinsing with ethyl acetate (3 mL). The solvent was evaporated to give a dark green solid (599 mg). Ethyl acetate (8 mL) was added to the green solid but did not dissolve it, so methanol (1 mL) was added and the mixture dissolved. Hexanes (90 mL) were added dropwise with stirring and 1 h after complete addition the liquid was decanted. The solid precipitate was rinsed with hexanes (4×3 mL) and dried on the vacuum pump for 1 h to give a dark green solid (409 mg). The solid was dissolved in 8:1 ethyl acetate:methanol (2 mL) and hexanes (20 mL) were added dropwise with stirring. One hour after complete addition the liquid was decanted and the residue rinsed with hexanes (4×3 mL). The residue was dried on the vacuum pump for 3 h to give a dark green solid (371 mg).

### Results

Direct Isolation of a Carotenoid-Oxygen Copolymer Compound from Carrot Powder.

Given the high level of OxPVA estimated from the GA present in carrot powder (ca. 0.5 mg/g in carrot powder #1), and knowing that the OxBC polymer from β-carotene is polar and insoluble in non-polar solvents, direct isolation of a carotenoid-oxygen copolymeric product by solvent precipitation was attempted. Indeed, addition of hexanes to an ethyl acetate extract of carrot powder #1 yielded a brown precipitate. Redissolving the recovered solid in ethyl acetate and precipitating again with hexanes and repeating the procedure gave a brown solid, which, at ca. 0.7 mg/g, was about 1.4 times the estimated OxPVA level, well within the anticipated range for the GA-based estimate (see Isolated Polymer and Polymer/OxPVA columns in Table 2). A similar ratio (1.6) was found for carrot powder #2, for which the yield of copolymer was around half that of carrot powder #1, in line with the respective relative amounts of GA. Confirmation that the isolated solid is largely composed of carotenoid-oxygen copolymer products was provided by comparison of elemental composition, IR, UV-Vis, GPC and GC-MS thermal decomposition data with the corresponding data for the OxBC polymer.

Elemental analyses for carbon, hydrogen, oxygen and nitrogen of the products isolated from extracts of carrot powders #1 and #2 confirmed they are comprised almost entirely of carbon, hydrogen and oxygen, with a trace of nitrogen, and marked by a high level of oxygen (ca. 24%, Table 5). The elemental C, H, and O empirical formulae calculated relative to the molecular formula for β-carotene (C₄₀H₅₆) are shown in Table 4. The results, C₄₀H₆₄O₁₁ and C₄₀H₆₈O₁₁ for carrot powders #1 and #2, respectively, are consistent with the addition, on average, of ca. 5-6 O₂ molecules to each carotene molecule. This number is somewhat less than the ca. 7 O₂ for the OxBC polymer or for oxidation of solid β-carotene in air (Table 4). It is possible differences in the reaction conditions and the presence of α-carotene, with one less conjugated double bond, result in reaction with fewer oxygen molecules. Interestingly, the carrot powder empirical formulae are very similar to that of sporopollenin isolated from Lycopodium clavatum (Table 4). Sporopollenin biopolymers, an integral component of the protective outer coatings of pollen and spores, have been proposed to be formed by carotenoid-oxygen copolymerization.²⁰

The IR spectrum of carrot powder extract shows a high degree of similarity to that of OxBC (FIG. 7). Previously we noted that the IR spectra of OxBC and Lycopodium clavatum sporopollenin also are strikingly similar.¹³

The UV-Vis spectrum of carrot powder extract shown in FIG. 8 is very similar to that of the OxBC polymer. Both spectra are characterized by a peak at ca. 205 nm and two broad shoulders at ca. 235 and 280 nm. These absorptions are consistent with the presence of carboxyl (205 nm), α,β-unsaturated carbonyl^{21, 22} (235 nm) and conjugated dienone²³ (280 nm) groups in the copolymers. The relative intensities of these absorptions will vary depending on the relative abundances of the associated functional groups, which can account for the small differences in the absorption profiles of OxBC and carrot powder #1 seen in FIG. 8.

The GPC molecular weight profile of the predominantly polymeric OxBC has been described previously.¹³ The polymeric nature of the carrot powder extract is illustrated in the GPC trace shown in FIG. 9. Comparison with the single, broad symmetric peak of the overlaid trace for the OxBC polymer shows the carrot powder product to be more complex. In addition to the two peaks that broadly coincide with the single OxBC polymer peak, there is an earlier eluting, broad peak indicating the presence of a higher molecular weight polymeric component. A UV-Vis cross-sectional analysis of the carrot powder GPC chromatogram vs. elution time indicates a degree of uniformity across the peaks that is consistent with them being essentially made up of carotenoid-oxygen copolymers. The same general UV-Vis spectral profile described above was apparent throughout, with changes in intensity displayed mostly in the 235 nm absorption region (data not shown).

The greater molecular weight spread of the carrot powder copolymers was attributed to the heterogeneous nature of the carrot matrix environment in which oxidation occurs. Whereas oxidation of β-carotene to form OxBC involves just β-carotene and oxygen in a solvent, oxidation in a carrot matrix occurs in the presence of additional molecular species (including α-carotene) and likely takes place within heterogeneous environments that can include emulsions, micelles and membranes. Radical autoxidation reactions in emulsions can proceed with longer chain lengths before radical-radical termination occurs, resulting in higher molecular weight polymers.

The thermal breakdown of the carrot powder extract into identifiable low molecular weight products also supports the carotenoid-oxygen copolymer nature of the compound. For comparison, injection of the OxBC polymer fraction (i.e., minus the low MW norisoprenoids) into the heated injector port of the GC-MS instrument results in rapid, thermal decomposition into numerous low MW compounds, some of which can be identified by retention times and comparisons of mass spectra to reference database information. Six compounds with a better than 50% match with the MS database¹⁹ were readily identified in the breakdown products. These include the well-known norisoprenoids, β-cyclocitral, dihydroactinidiolide, 4-oxo-β-ionone and 5,6-epoxy-β-ionone (FIG. 10). The presence of unsaturated carbonyl groups in these products is reflective of the presence of the same and related groups in the original precursor copolymers. The same six compounds also are present in the GC-MS of carrot powder #2 (FIG. 10). A seventh product, identified as α-ionone, likely originates from α-carotene copolymers.

Isolation of Carotenoid-Oxygen Copolymer Compounds from Other Foods

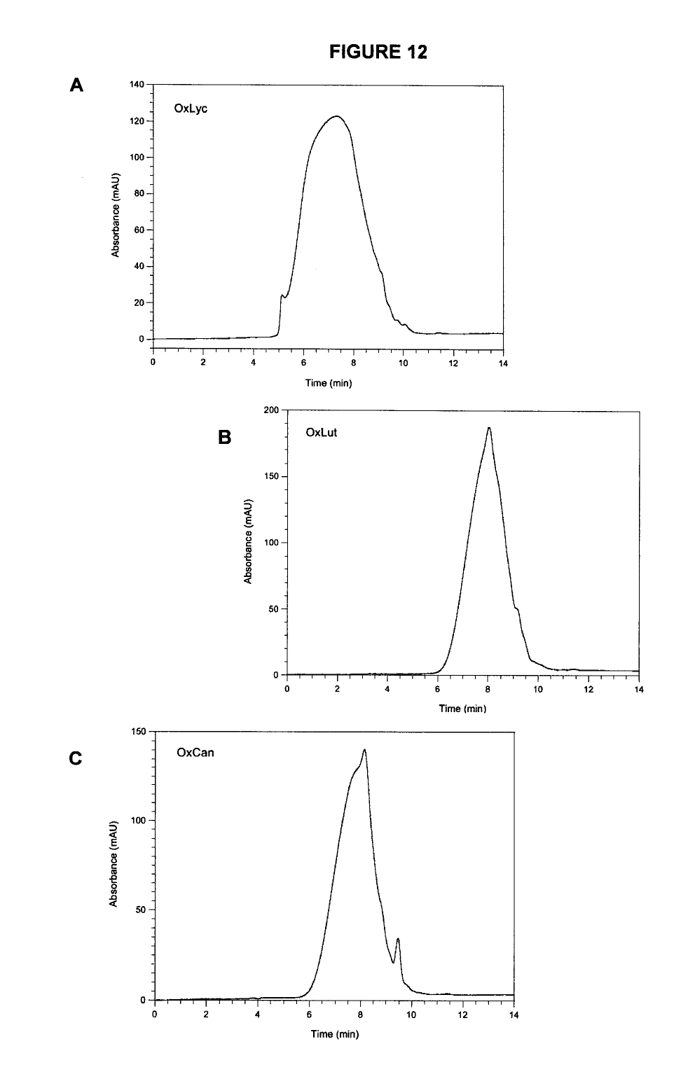

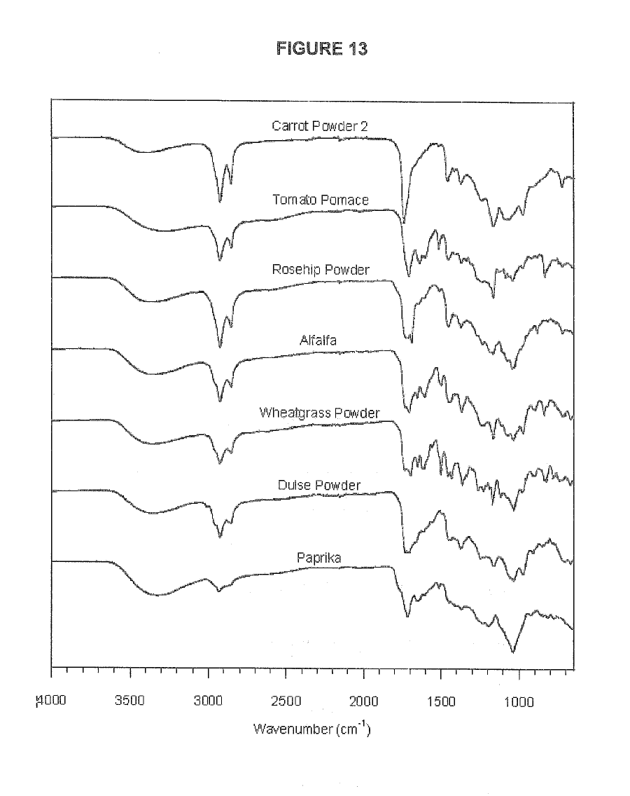

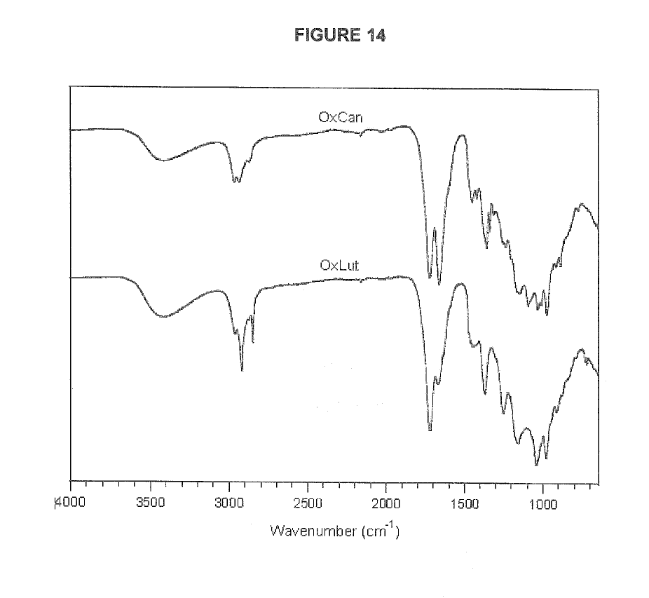

Solids containing substantial quantities of polymeric material were readily isolated by hexane precipitation of ethyl acetate extracts of tomato powder, tomato pomace, rosehip powder, paprika, dulse seaweed powder, sun-cured alfalfa and wheatgrass powder (see the last 2 columns of Table 2). Elemental analyses confirmed the compounds as comprised essentially of carbon, hydrogen and oxygen, with a minor amount of nitrogen (1-2%), and marked by a high content of oxygen (21-39%) (Table 4 and Table 5). GPC analyses confirmed the polymeric nature of the compounds (FIG. 11) and indicate a more complex nature in comparison to the copolymers obtained from oxidation of single pure candidate carotenoids in ethyl acetate (FIGS. 9 and 12). The corresponding FTIR spectra (FIGS. 7 and 13) show a high degree of similarity both with each other as well as with the spectra of fully oxidized β-carotene and lycopene (FIG. 7) and fully oxidized lutein and canthaxanthin (FIG. 14).

Comparison of the yields of isolated polymer with corresponding estimated OxPVA levels, calculated as the Polymer/OxPVA ratio in Table 2, shows values many-fold greater than the value of ˜1 for carrot powder. This reflects the abundance of other carotenoids, including lycopene, lutein and capsanthin, which also participate in oxidative polymerization to give product at parts-per-thousand levels (e.g., tomato and rosehip powders and paprika).

In the example of tomato powder lycopene is the dominant carotenoid. As already reported,¹³ lycopene reacts even more rapidly than β-carotene with oxygen, forming higher molecular weight copolymers in apparently even greater amount. The empirical formula in Table 4 for OxLyc indicates the addition, on average, of 7-8 O₂ per lycopene. The corresponding data for the tomato powder extract show enhanced uptake of oxygen (7-8 O₂) compared to carrot powder, with a C, H, O empirical formula (C₄₀H₆₂O₁₅) similar to that of Lilium henryii sporopollenin (Table 4).

Table 2 shows the polymeric product isolated from tomato powder exceeds the estimated OxPVA level by more than 100-fold. Although other contaminating compounds could be present, it is known lycopene can exceed β-carotene by such a range in processed tomato products.²⁴ The high degree of similarity of the IR spectrum (FIG. 7) to the spectra of OxLyc, OxBC and carrot powder copolymer suggests that levels of contaminating compounds are not significant.

### Example 4—Geranic Acid as a Marker of Autoxidation of Lycopene and γ-Carotene

This example shows that geranic acid (I) can be a marker of autoxidation of lycopene and γ-carotene.

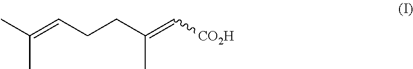

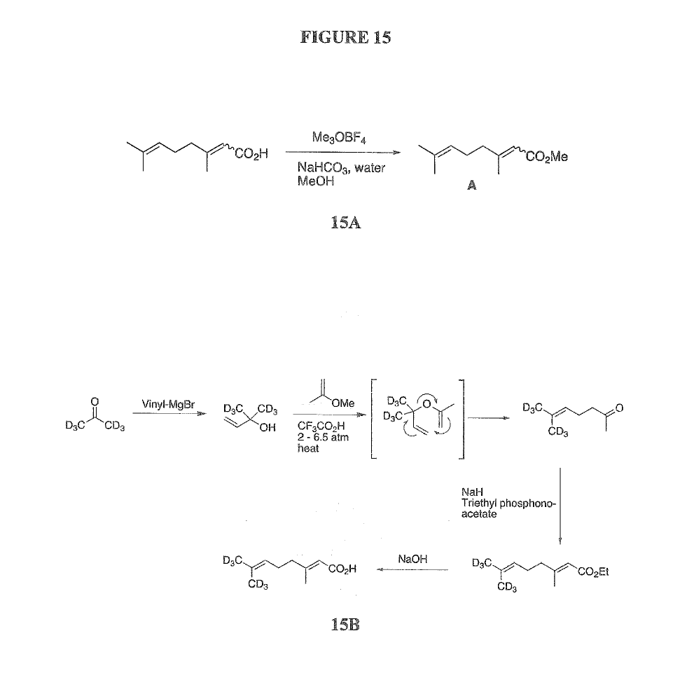

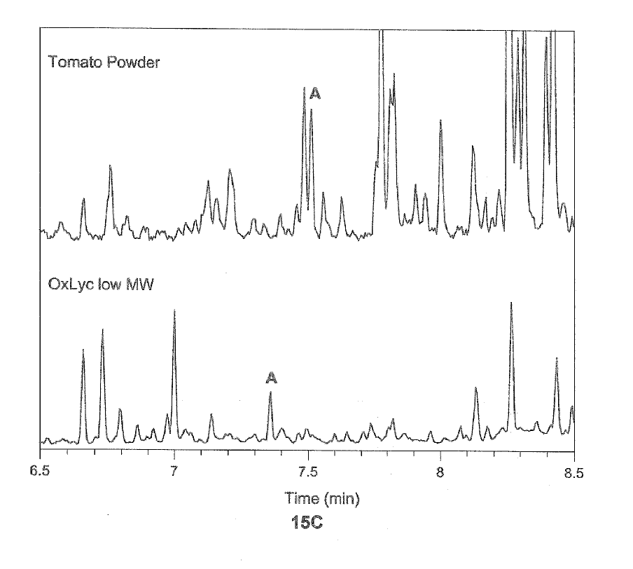

This marker compound was identified in fully autoxidized lycopene (OxLyc) and is also expected to be present in fully autoxidized γ-carotene. The inventors identified same in the low MW fraction of OxLyc after removal of the polymer fraction by solvent precipitation. Identification was made by GC-MS, where a 39% match was found with the mass spectral library. Isolation of the acid fraction of OxLyc by extraction with aqueous Na₂CO₃, followed by acidification, with aqueous HCl and then extraction with diethyl ether and injection into the GC-MS gave geranic acid with an 80% GC-MS library match. A reference standard was purchased from Aldrich to compare and confirm the structure by GC-MS (87% library match). Esterification of the acid with Me₃OBF₄ yielded methyl geranate (see FIG. 15A; 83% GC-MS library match); subjecting the low MW fraction of OxLyc to the same esterification conditions also gave methyl geranate (76% GC-MS library match).

FIG. 15A illustrates the esterifcation of geranic acid with Me₃OBF₄ to give methyl geranate (compound A). A proposed synthesis of deuterium-labeled geranic acid is provided in FIG. 15B. This compound could be used as an internal standard for measuring the amount of geranic acid in foods, thus providing an estimate of the amount of oxidized lycopene and, indirectly, its associated copolymers. Whereas one equivalent of lycopene could generate two equivalents of geranic acid, one equivalent of γ-carotene should generate only one equivalent of geranic acid.

Samples of tomato powder and tomato pomace extract, esterified with Me₃OBF₄, showed the presence of methyl geranate (45% and 65% library matches, respectively). FIG. 15C shows the GC chromatograms of esterified tomato powder extract (top) and OxLyc low MW fraction (bottom), with methyl geranate indicated as compound A. The difference in retention times is the result of some small differences in the analysis conditions, for instance due to the GC column being trimmed between samples, resulting in a shorter retention time for the OxLyc low MW run. GC-MS analysis of esterified rose hip extract also revealed the presence of methyl geranate (75% library match.

### Example 5—Enhancing Carotenoid-Oxygen Copolymers in Foods

This study was designed to illustrate that factors known to increase oxidation would increase the levels of carotenoid-oxygen copolymers in foods. It is already known that geronic acid serves as an indicator of carotenoid-oxygen copolymer content (Burton et al.⁵³). This study makes use of that fact by measuring the increase in geronic acid in carrots that have been exposed to conditions expected to increase oxidation. Those conditions include increased surface area (puréeing, powdering), dehydration, heat and light.

### Experimental Design

Fresh, peeled carrots with tops and bottoms cut off were rinsed with water, patted dry with a paper towel and finely shredded with a food processor. Carrot shreds were analyzed for geronic acid to determine the content in fresh carrot (see Assay Methods below for the detailed procedure). Shreds were also puréed using a food processor, spread approximately ¼ inch thick onto parchment paper, dehydrated in a food dehydrator (Excalibur 3926 TB) for 10 hrs at ca. 52-53° C., then allowed to cool to room temp for ca. 8 hrs. The dried purée was analyzed for geronic acid and β-carotene content (see Assay Methods below for detailed procedures). The remaining dried purée was powdered using a food processor blade, then sifted through a kitchen sieve to remove large particles. The carrot powder was spread thinly onto a tray (46 cm×36 cm) lined with aluminum foil and placed in an open space. Illumination with a fluorescent light approximately 1.1 meters above the tray was used to approximately simulate exposure to ambient light conditions. Samples of carrot powder were taken at intervals 5-9 days apart for analysis of geronic acid and β-carotene content (see Assay Methods below for detailed procedures). Before taking samples of the powder, the tray was gently shaken to mix the powder on the top with that lying beneath it.

### Assay Methods

Geronic Acid Assay of Fresh Carrot

Fresh, peeled carrots with tops and bottoms cut off were rinsed and patted dry with paper towel, then finely shredded using a food processor. 230-400 g of carrot shreds were placed in a 1 L beaker, followed by 0.4 mL of geronic acid-d₆ solution (0.001604 mg/mL in methanol), BHT (butylated hydroxytoluene; 4-5 mg), and chloroform (CHCl₃) (400-500 mL). The mixture was homogenized for 20 min at 13,500 rpm, then the homogenizer was shut off and the liquid allowed to drain into the beaker, rinsing the homogenizer inside and outside with CHCl₃ (total of 4×1.5 mL). All material was transferred to a 2 L separatory funnel, the orange CHCl₃ layer was separated from the orange pulp, and the pulp was returned to the beaker. CHCl₃ (300-400 mL, containing 2-4 mg BHT) was added to the pulp and homogenized for 5 min. The CHCl₃ layer was separated as before, the CHCl₃ extracts were combined, and solvent was evaporated on the rotavap. CHCl₃ was added (15 mL) to dissolve the residue, and the mixture was dried (MgSO₄) and filtered through cotton, rinsing with CHCl₃ (total 15 mL). Aqueous KOH (25 mL; prepared by dissolving ˜90 mg KOH in 50 mL water) was added to the CHCl₃ solution and stirred vigorously for 5 min. The mixture was transferred to a separatory funnel, most of the CHCl₃ was removed, and the remaining liquid was centrifuged for 5 min. The aqueous layer was separated, acidified (˜3 mL of 1 M HCl), and extracted with CHCl₃ (2×15 mL). The combined extracts were dried (Na₂SO₄), filtered and solvent evaporated. The residue was dissolved in methanol (9 mL), solid NaHCO₃ (˜0.1 g) was added and the mixture stirred. Aqueous NaHCO₃ (1 mL, 1 M) was added, followed by Me₃OBF₄ (ca. 0.3 g). After 15 min stirring, 9 mL water (H₂O) was added, stirred, and the mixture extracted with dichloromethane (CH₂Cl₂) (2×7.5 mL). The combined CH₂Cl₂ extracts were dried with Na₂SO₄, filtered through cotton and solvent carefully evaporated on the rotary evaporator at room temperature. The residue was dissolved in 0.2 mL acetonitrile and 1 μL injected into the GC-MS (splitless injection, SIM mode monitoring ions 154.1 and 160.1).

Geronic Acid Assay of Dried Carrot Purée or Powder

All ethyl acetate used in this procedure contained 0.05 mg/mL BHT. Approximately 3.5 g of dried carrot purée or powder was weighed in a 50 mL test tube (carrot purée was crushed with a spatula to fit it into the bottom of the tube). To the tube was added 15 mL ethyl acetate and geronic acid-d₆ (0.64-13 μg in methanol). The mixture was homogenized for 10 min at 13,500 rpm, then 10 min at 6,500 rpm. The homogenizer was shut off and the liquid allowed to drain into the tube, rinsing the homogenizer inside and outside with ethyl acetate (total of 4×1.5 mL). All material was transferred to 2×15 mL centrifuge test tubes and centrifuged (˜5 min). The supernatant was transferred to a 50 mL round bottom flask and solvent was evaporated. At this point the flask was sealed under argon and stored overnight in the freezer until the next morning. Then, 6 mL of 5% aqueous NH3 and 3 mL H₂O were added and stirred vigorously for 20 min. In the meantime, SPE cartridges (Waters Oasis MAX, 500 mg/6 mL) were prepared by passing through the following solutions in sequence: methanol (6 mL), H₂O (6 mL), 0.5% aqueous NH₃ (4.5 mL). The basic solution of analyte was then passed through the cartridge by gravity (pressure with a pipette bulb is acceptable if the flow is very slow). The cartridge was then washed with 0.5% aqueous NH₃ (4.5 mL), followed by methanol (9 mL). Carboxylic acids were then eluted from the cartridge by passing through a solution of 2% HCl in methanol (4.5 mL) and collected in a 20 mL scintillation vial. Solid NaHCO₃ was added and stirred until bubbling ceased (˜30 sec). Then, 1 M NaHCO₃ (1 mL) was added, giving a cloudy solution. The solution was stirred gently while Me₃OBF₄ was added (ca.0.3 g). The mixture was stirred vigorously for 15 min and maintained slightly basic by ensuring the presence of a small amount of solid NaHCO₃ in the vial (visual inspection—adding more if necessary). After 15 min, 6 mL H₂O was added, stirred, and the mixture extracted with CH₂Cl₂ (2×6 mL). The combined CH₂Cl₂ extracts were dried with Na₂SO₄, filtered through cotton and the solvent carefully evaporated on the rotary evaporator at room temp. The residue was dissolved in 0.2 mL acetonitrile, filtered if necessary through a 0.2 μm Teflon syringe filter, and 1 μL injected into the GC-MS (splitless injection, SIM mode monitoring ions 154.1 and 160.1).

β-Carotene Assay of Dried Carrot Puree or Powder

All ethyl acetate used in this procedure contained 0.05 mg/mL BHT. Approximately 1.0 g of dried carrot purée or powder was weighed in a 50 mL test tube. To the tube was added 20 mL ethyl acetate. It was homogenized for 10 min at 13,500 rpm, then the homogenizer was shut off and the liquid allowed to drain into the tube, rinsing the homogenizer inside and outside with ethyl acetate (5×1.5 mL). All material was transferred to 2×15 mL centrifuge test tubes, centrifuged for ˜5 min, and the supernatant transferred to a 100 mL volumetric flask. The residue was transferred back to the 50 mL test tube, rinsing the centrifuge tubes as needed with ethyl acetate to ensure complete transfer. A total of 20 mL ethyl acetate was added to the residue and homogenized for 3 min at 6,500 rpm. The mixture was centrifuged and separated as before, and the residue extracted once more (3 min, 6,500 rpm). The liquid from all 3 extractions were combined into the 100 mL volumetric flask, diluted to volume with ethyl acetate and inverted 30 times to mix. 1 mL of this cloudy orange solution was filtered through a 0.2 m Teflon syringe filter into a 1 mL volumetric vial. The solution was transferred by pipette to a 10 mL volumetric flask, rinsing carefully with several portions of ethyl acetate to ensure complete transfer. The solution was made up to the 10 mL mark with ethyl acetate, inverted 30 times to mix and the absorbance of the solution was measured at 454 nm. Using the previously determined β-carotene extinction coefficient of 237.67 mL·mg-1·cm-1, the amount of β-carotene in solution was calculated.

### Results and Discussion

Fresh, shredded carrots were found to contain 4.9 ng/g geronic acid (Table 6). Dehydration of the carrot purée resulted in a mass decrease of 89.5%. As seen in Table 6, the amount of geronic acid in the processed carrot at day 0 had increased approximately 20-fold after puréeing and dehydration. The increased level of geronic acid is a result of both the 10-fold decrease in mass during dehydration and of some oxidation. Further processing by powdering the dried purée and spreading it thinly on a tray to expose it to air and light caused an approximate 9-fold increase in geronic acid after 5 days, with a concomitant decrease in β-carotene (Table 6). After 21 days, geronic acid has increased substantially to 4155 ng/g, and β-carotene had decreased to less than half of the starting amount.

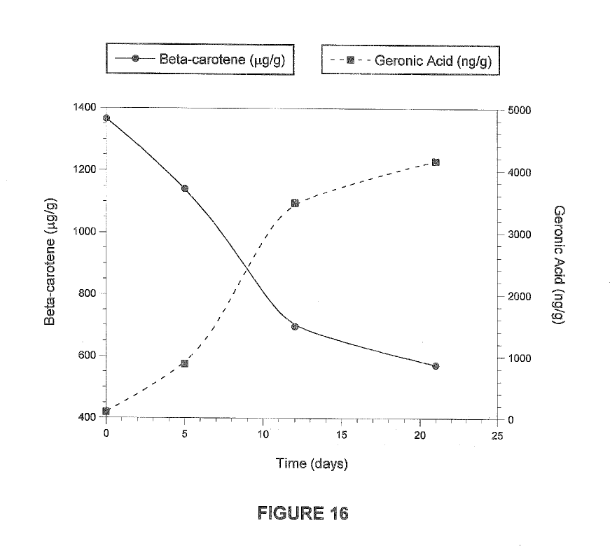

FIG. 16 shows graphically the inverse relationship of the rise of geronic acid to the decrease of β-carotene. Dividing the change in geronic acid by the change in β-carotene at each time point gives a ratio that is roughly constant (Table 6), indicating that production of geronic acid is closely linked to the oxidative loss of β-carotene.

Table 6 shows the effect of processing upon geronic acid and β-carotene levels in carrots.

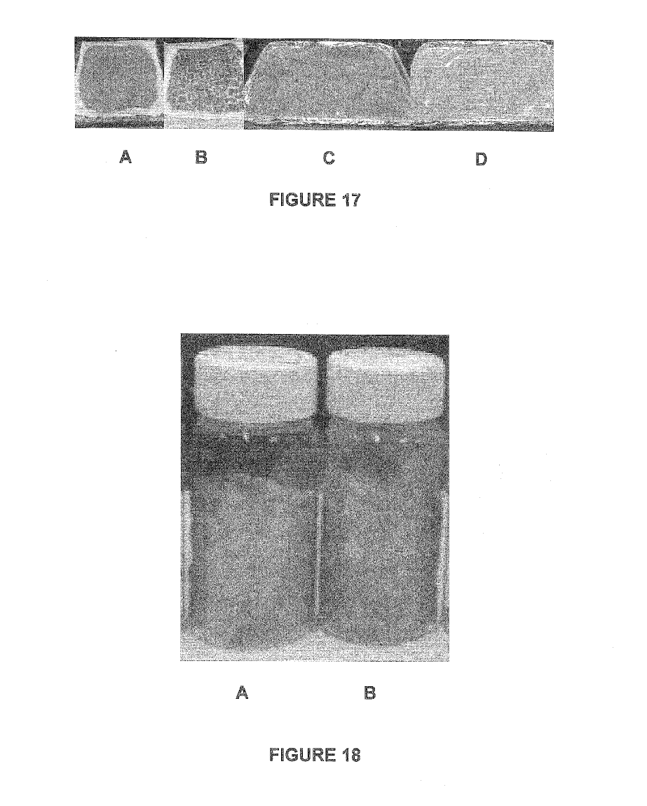

Images of the processed carrot as it progressed through various stages of dehydration are shown in FIG. 17. The original carrot purée is bright orange, as are the dehydrated purée and powder 1 day later, but over time the orange colour fades, as seen in the carrot powder picture taken after 21 days. The loss of colour is indicative of β-carotene oxidation and the associated geronic acid and, indirectly, carotenoid-oxygen copolymer formation.

A visual illustration of the importance of air exposure and the physical state of the carrot in enhancing oxidation is shown in FIG. 18, which contrasts two vials of carrot powder. The vial of orange powder on the left was prepared by dehydrating carrot chips (cut ˜¼ inch thick from fresh carrots), grinding them in a coffee blade mill, then allowing it to stand in a sealed vial for 4 weeks and 6 days.

The much finer, light brown powder in the vial on the right was prepared by dehydrating carrot purée, powdering it with a food processor, then grinding the powder to a consistent size with a Baratza Virtuoso Coffee Burr Mill. The powder was ground at the coarsest setting of 40, then again at 30, and finally at 20. The powder was placed on a tray lined with aluminum foil and exposed to air for 1 week and 6 days, with no attempt to exclude light.

The carrot powder sealed in the vial on the left is still bright orange after 4 weeks and 6 days, but the powder in the vial on the right that was exposed to air for just 1 week and 6 days is light brown, indicating a much greater loss of β-carotene.

This experiment illustrates the importance of a finely divided carrot powder and ready access to air to enhance the extent of β-carotene oxidation.

### Example 6—4-Hydroxygeronic Acid and its Lactone-Markers of Autoxidation of Lutein, Zeaxanthin and Capsanthin

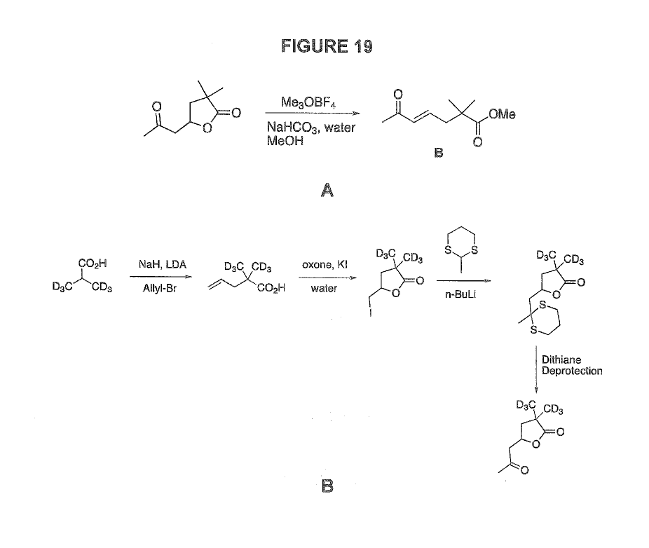

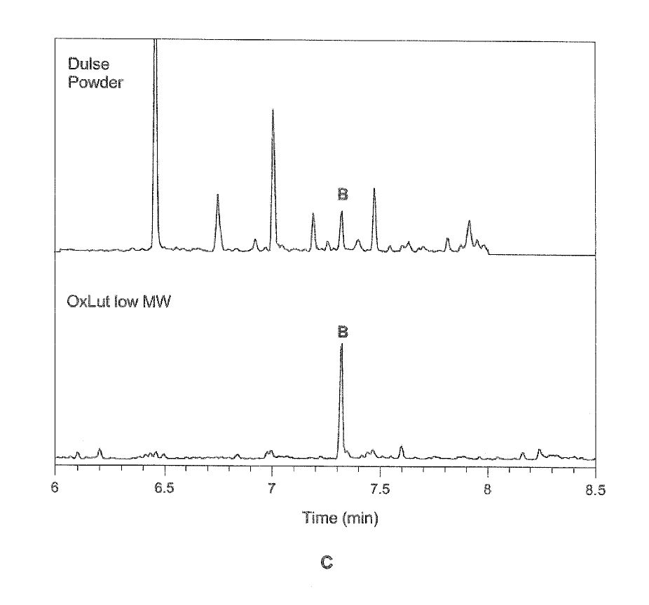

4-hydroxygeronic acid and its lactone are markers for fully oxidized lutein or zeaxanthin and, in principle, capsanthin. The lactone was isolated from the ozonolysis of lutein and identified by NMR (¹H, ¹³C), Electrospray MS and GC-MS. The low MW liquid fraction of OxLut, obtained after removal of the polymer fraction by solvent precipitation, contained the lactone, as confirmed by GC-MS. Extracting the low MW liquid fraction of OxLut with aqueous Na₂CO₃, followed by acidification with aqueous HCl and extraction with diethyl ether gave the lactone upon GC-MS analysis. When subjected to esterification with Me₃OBF₄, the lactone opens up and dehydrates to give 4,5-didehydromethyl geronate (compound B, FIG. 19A). Compound B was confirmed by 1H NMR and GC-MS analysis.

FIG. 19A illustrates the formation of 4,5-didehydromethyl geronate (compound B) by reaction of its parent lactone with Me₃OBF₄. Compound B is formed in OxLut low MW liquid fraction upon esterification with Me₃OBF₄. It is also present in similarly esterified extracts of dulse powder, nori flakes and Greens+ powder (a dietary supplement), by comparison of GC retention times and mass spectra. FIG. 19C shows the GC chromatogram of esterified extracts of dulse powder and the OxLut low MW fraction. Esterification of paprika extract, a source of capsanthin, however did not reveal the presence of compound B.

A proposed synthesis of deuterium labeled lactone is provided in FIG. 19B. This compound could be used as an internal standard for measuring the amount of lactone in foods, thus providing an estimate of the amount of oxidized lutein or zeaxanthin, and, indirectly, the associated copolymers. Preparation of isobutyric acid-d₆ starting material is described in Burton et al.¹³, in Can. J. Chem., 92, 305-316 (2014).

FIG. 19C is a GC chromatograms of dulse powder extract and low MW fraction of OxLut, esterified with Me₃₀OBF₄. Compound B is 4,5-didehydromethyl geronate, with a retention time of 7.32 min. Common mass spectral ions include m/z=184 (M+), 152, 125, 109, 83, 81, 69, 55, 43.

### Example 7—2,2-Dimethylglutaric Acid and its Anhydride-Markers of Autoxidation of Canthaxanthin

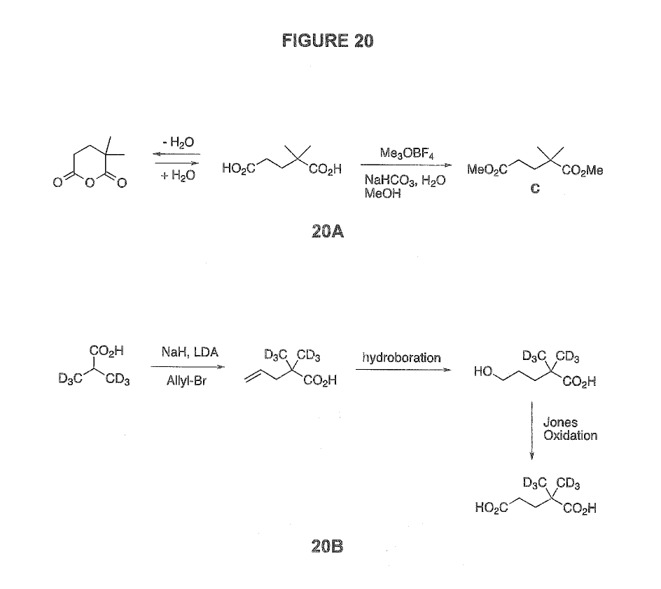

2,2,-dimethylglutaric acid and its anhydride are potential markers for fully oxidized canthaxanthin. They were isolated from the ozonolysis of canthaxanthin and identified by ¹H NMR and GC-MS. The low MW liquid fraction of OxCan, obtained after removal of the polymer fraction by solvent precipitation, contained the anhydride, as confirmed by GC-MS (69% mass spectral library match). The reaction products of ozonolysis of canthaxanthin were subjected to esterification with Me₃OBF₄, providing dimethyl 2-2-dimethylglutarate (compound C, FIG. 20A). Compound C was isolated and its structure confirmed by ¹H NMR and GC-MS. Esterification of the low MW liquid fraction of OxCan with Me₃OBF₄ also gave compound C (91% library match). FIG. 20A illustrates the conversion of 2,2-dimethylglutaric acid to its anhydride and its dimethyl ester, compound C.

A proposed synthesis of deuterium labeled 2,2-dimethylglutaric acid is given in FIG. 20B. This compound could be used as an internal standard for measuring the amount of 2,2-dimethylglutaric acid in foods, thus providing an estimate of the amount of oxidized canthaxanthin and, indirectly, its associated copolymer. Preparation of isobutyric acid-d₆ starting material of FIG. 20B is described by Burton et al.¹³, Can. J. Chem., 92, 305-316 (2014).

### TABLES

TABLE 1A
Concentration of geronic acid, GA, in carrot juice
determined by GC-MS using a deuterium-labeled GA
internal standard. Comparison of direct measurement
vs. purification via semicarbazone derivative, and
effect of added antioxidant.

| | Sample | Intensity | Mass | |
|---|---|---|---|---|
| | Weight^{a} | Ratio | Ratio^{b} | GA^{c} |
| Sample | W (g) | I/I₆ | m/m₆ | (ng/g) |
| 1 | 201 | 1.319 | 1.230 | 18.1^{d} [17.7^{d}] |
| 2 | 197 | 1.196 | 1.121 | 16.6^{e} [16.5^{e}] |
| 3 | 202 | 0.898 | 0.855^{f} | 12.3 |
| 4 | 194 | 0.926 | 0.880^{f} | 13.2 |
| 5 | 183 | 0.879 | 0.838^{f} | 13.3 |
| 6 | 173 | 0.670 | 0.684^{g} | [11.5] |

| | |
|---|---|
| Mean (samples 3-6): | 12.6 ± 0.8 |

^{a}Amount of added GA-d₆ internal standard, m₆, = 2.9 μg.
^{b}Calculated from the measured intensity ratio, I/I₆, using equation 1.
^{c}Values in square brackets obtained via semicarbazone derivative.
^{d}No BHT added, sample preparation time 32 h.
^{e}No BHT added, sample preparation time 8 h.
^{f}Calibration: I/I₆ = 1.121 m/m₆ − 0.029; R² = 0.996.
^{g}Calibration: I/I₆ = 1.025 m/m₆ − 0.031; R² = 0.999.

TABLE 1B
Concentration of GA in raw tomato.

| | Sample | Intensity | Mass | |
|---|---|---|---|---|
| | Weight^{a} | Ratio | Ratio^{b} | GA |
| Sample | W (g) | I/I₆ | m/m₆ | (ng/g) |
| 1 | 197 | 0.103 | 0.055 | 0.78 |
| 2 | 201 | 0.124 | 0.075 | 1.03 |
| 3 | 217 | 0.179 | 0.126 | 1.62 |
| 4 | 236 | 0.322 | 0.258 | 3.05 |
| 5 | 241 | 0.135 | 0.085 | 0.98 |
| | | | Mean: | 1.5 ± 0.9 |

^{a}Amount of added GA-d₆ internal standard, m₆, = 2.8 μg.
^{b}Calculated from the measured intensity ratio, I/I₀, using equation 1. Calibration: I/I₆ = 1.080 m/m₆ + 0.044; R² = 0.999.

TABLE 2
Measured concentrations in foods of geronic acid, GA, arising from oxidation of provitamin A carotenoids (PVA).
GA values provide estimates of total PVA oxidation products, OxPVA, that may be compared to literature or estimated
PVA levels and to levels of carotenoid-oxygen copolymer products isolated from ethyl acetate extracts of some
powdered foods.

| | | | | | OxPVA/ | Isolated | |
|---|---|---|---|---|---|---|---|
| | | GA | OxPVA^{a} | PVA^{b,c} | PVA^{d} | Polymer^{e} | Polymer/ |
| Sample | n | (ng/g) | (μg/g) | (μg/g) | (%) | (μg/g) | OxPVA^{f} |
| Carrot juice | 4 | 12.6 ± 0.8 | 0.63 | 136 | 0.5 | | |
| Carrot powder #1^{g} | 3 | 10,590 ± 550 | 530 | 965 [118] | 55 | 756 | 1.4 |
| Carrot powder #2^{h} | 3 | 5007 ± 119 | 250 | 965 [118] | 21 | 404 | 1.6 |
| Tomato, raw | 5 | 1.5 ± 0.9 | 0.08 | 5.5 | 1.4 | | |
| Tomato powder | 3 | 414 ± 46 | 21 | 97 | 21 | 2600 | 126 |
| Tomato pomace | 3 | 113 ± 3 | 6 | 90 | 6 | 1006 | 178 |
| Cranberry, raw | 1 | 3.8 | 0.19 | 1.6 | 12 | | |
| Cranberry powder | 3 | 338 ± 55 | 17 | 12 | 142 | | |
| Rosehip powder | 4 | 499 ± 12 | 25 | 66 [29] | 38 | 1380 | 55 |
| Paprikaⁱ | 3 | 364 ± 22 | 18 | 243 [21] | 7 | 1080 | 59 |
| Spirulina powder | 3 | 2560 ± 10 | 128 | 14,303 [1400] | 1 | | |
| Sweet potato powder #1^{j} | 3 | 692 ± 22 | 35 | 345 [85] | 10 | | |
| Sweet potato powder #2^{k} | 3 | 417 ± 55 | 21 | 352 [85] | 6 | | |
| Dulse seaweed powder | 3 | 1603 ± 39 | 80 | 194 [31] | 41 | 634 | 8 |
| Nori seaweed flakes | 3 | 2002 ± 33 | 100 | 198 [31] | 51 | | |
| Dates, dried | 7 | 32 ± 12 | 1.6 | 0.54 [0.37] | 296 | | |
| Alfalfa (sun-cured) | 2 | 869 ± 37 | 43 | 643 [148] | 7 | 978 | 23 |
| Wheatgrass powder | 3 | 964 ± 7 | 48 | 231 [42] | 21 | 924 | 19 |
| Red palm oil | 3 | 60 ± 1 | 3.0 | 506 | 0.6 | | |
| Milk (3.25% MF) | 2 | 6.7 ± 2.1 | 0.34 | 0.07 | 479 | | |
| Milk powder (3.25% MF) | 3 | 2.0 ± 0.1 | 0.10 | 0.58 | 17 | | |
| Whole egg powder | 3 | 34 ± 2 | 1.7 | 0.37 [0.09] | 463 | | |

^{a}Estimated approximate total amount of provitamin A carotenoid oxidation products, OxPVA, = 50 × GA, assuming GA production parallels that for the model ß-carotene oxidation in solution. OxPVA implicitly includes any, mostly minor, contributions from two other provitamin A carotenoids, α-carotene and cryptoxanthin (Table 3), which can in principle each contribute one molecule of GA per carotenoid molecule, compared to two from ß-carotene.
^{b}Sum of provitamin A carotenoid levels, PVA = α- + ß-carotenes + cryptoxanthin, using literature values for raw food and expressed as nominal original amounts for dehydrated forms after adjusting for water losses (Table 3). γ-carotene may also contribute very minor amounts of GA to OxPVA calculations.
^{c}Values in square brackets are for parent raw form.
^{d}Estimated total provitamin A carotenoid-oxidation products, OxPVA, as a percentage of the sum of literature-based initial provitamin A carotenoid levels, PVA.
^{e}Weight (μg) of carotenoid-oxygen copolymer fraction per gram of dehydrated food isolated by successive precipitations from ethyl acetate extract with hexane.
^{f}Ratio of isolated polymer fraction to OxPVA.
^{g}Light brown powder.
^{h}Orange powder.
ⁱComparison with raw, sweet red pepper.
^{j}Drum-dried.
^{k}Air-dried.

TABLE 3
Water and approximate provitamin A carotenoid levels of various foods. Nominal, starting carotenoid values in dried
foods were estimated by taking literature values for corresponding raw food and adjusting for water lost during drying.*

| | | Provitamin A Carotenoids (PVA; μg/g) | | | Total PVA | |
|---|---|---|---|---|---|---|
| Sample | % Water | ß-Carotene | α-Carotene | Cryptoxanthin | (μg/g) | Literature Sources |
| Carrot juice | 89 | 93 | 43 | | 136 | 32a |
| Carrot powders #1 and #2 | 4 [88] | 680 [83] | 285 [35] | | 965 [118] | 32b, 32c |
| Tomato, raw | 95 | 4.5 | 1.0 | | 5.5 | 32d |
| Tomato powder | 3 | 79 | 18 | | 97 | 32d, 33a |
| Tomato pomace | 10 | 74 | 16 | | 90 | 32d, 34 |
| Cranberry, raw | 87 | 0.36 | | | 0.36 | 32e |
| Cranberry powder | 4 | 2.7 | | | 2.7 | 32e, 43 |
| Rosehip powder | 6 [59] | 54 [24] | 0.70 [0.31] | 11 [4.8] | 66 [29] | 32f, 35 |
| Paprika | 11 [92] | 185 [16] | 2.3 [0.20] | 56 [4.9] | 243 [21] | 32g, 32h |
| Spirulina powder | 5 [91] | 14,303 [1400] | | | 14,303 [1400] | 32i, 32j, 36 |
| Sweet potato powder 1 | 8 [77] | 345 [85] | 0.28 [0.07] | | 345 [85] | 32k, 34 |
| Sweet potato powder 2 | 6 [77] | 352 [85] | 0.29 [0.07] | | 352 [85] | 32k, 34 |
| Dulse seaweed powder | 7 [85] | 194 [31] | | | 194 [31] | 33b, 33c |
| Nori seaweed flakes | 5 [85] | 198 [31] | | | 198 [31] | 33b, 33c, 34 |
| Dates, dried | 15 [42] | 0.50 [0.34] | 0.04 [0.03] | | 0.54 [0.37] | 37 |
| Alfalfa (sun-cured) | 7 [79] | 643 [148] | | | 643 [148] | 38, 39 |
| Wheatgrass powder | 5 [83] | 231 [41] | | | 231 [41] | 40, 41 |
| Red palm oil | | 264 | 242 | | 506 | 42 |
| Milk (3.25% milk fat) | 88 | 0.07 | | | 0.07 | 32l |
| Milk powder (3.25% MF) | 2.5 | 0.6 | | | 0.6 | 32m |
| Whole egg powder | 2.8 [76] | | | 0.37 [0.1] | 0.37 [0.1] | 32n, 32o |

*Values in square brackets are literature values for raw foods. Carotenoid levels in dried foods were calculated using the formula: Carotenoid (dried) = Carotenoid (raw) × (100 − % Water (dried))/(100 − % Water (raw))

TABLE 4
Empirical formulae, expressed relative to β-carotene
(C₄₀H₅₆), calculated from elemental analysis data
for fully oxidized β-carotene, lycopene and lutein, copolymers
isolated by solvent precipitation from extracts of selected dried
foods, and from data for various sporopollenins.^{a}

| Sample | C | H | O | N |
|---|---|---|---|---|
| Fully Oxidized Pure Carotenoids^{a}: | | | | |
| OxBC polymer | 40 | 56.5 | 14.7 | |
| β-carotene (solid) | 40 | 56.9 | 14.2 | |
| air oxidation | | | | |
| OxLyc polymer | 40 | 59.0 | 15.2 | |
| OxLut polymer | 40 | 55.8 | 15.4 | |
| OxCan polymer | 40 | 55.1 | 14.7 | |
| Polymeric Solids Isolated from Dried Foods^{a}: | | | | |
| Carrot powder #1 | 40 | 64.0 | 11.1 | 0.3 |
| Carrot powder #2 | 40 | 67.7 | 11.2 | 0.3 |
| Tomato powder | 40 | 61.5 | 15.2 | 1.2 |
| Tomato pomace | 40 | 65.2 | 12.5 | 0.9 |
| Rosehip powder | 40 | 67.1 | 10.6 | |
| Paprika | 40 | 64.7 | 22.6 | 1.4 |
| Alfalfa (sun-dried) | 40 | 58.9 | 10.3 | 0.7 |
| Wheat grass powder | 40 | 59.5 | 11.7 | 0.9 |
| Dulse seaweed powder | 40 | 58.9 | 9.7 | 1.3 |
| Sporopollenins^{b}: | | | | |
| Lycopodium clavatum | 40 | 64.0 | 12.0 | |
| Lilium henryii | 40 | 63.1 | 16.0 | |
| Pinus canadensis | 40 | 66.7 | 16.4 | |
| Pinus radiata | 40 | 66.2 | 19.6 | |
| Pinus silvestris | 40 | 70.2 | 19.6 | |

^{a}Elemental analysis data for copolymers from dried food extracts, OxLyc, OxLut and OxCan provided in Table 5.
^{b}Calculated from data for sporopollenins in Shaw,³¹ pp. 314-315.

TABLE 5
Elemental analyses for carbon, hydrogen, oxygen
and nitrogen of fully oxidized β-carotene, lycopene,
lutein and canthaxanthin, and precipitates obtained
from ethyl acetate extracts of various dried foods

| Sample | C | H | O | N | Total |
|---|---|---|---|---|---|
| OxBC | 62.09 | 7.36 | 30.33 | <0.3 | 99.78 |
| OxLyc | 60.97 | 7.55 | 30.95 | <0.3 | 99.47 |
| OxLut | 60.35 | 7.07 | 30.93 | <0.3 | 98.35 |
| OxCan | 61.88 | 7.16 | 30.28 | <0.3 | 99.32 |
| Carrot powder #1 | 65.73 | 8.83 | 24.24 | 0.51 | 99.31 |
| Carrot powder #2 | 66.07 | 9.39 | 24.61 | 0.54 | 100.61 |
| Tomato powder | 60.49 | 7.81 | 30.71 | 2.10 | 101.11 |
| Tomato pomace | 62.77 | 8.59 | 26.12 | 1.56 | 99.04 |
| Rosehip powder | 66.77 | 9.40 | 23.48 | <0.3 | 99.65 |
| Paprika | 51.53 | 7.00 | 38.84 | 2.15 | 99.52 |
| Alfalfa (sun-cured) | 66.41 | 8.21 | 22.84 | 1.43 | 98.89 |
| Wheatgrass powder | 64.50 | 8.05 | 25.22 | 1.71 | 99.48 |
| Dulse seaweed powder | 64.63 | 7.99 | 20.80 | 2.44 | 95.86 |

TABLE 6
Effect of processing upon geronic acid and β-carotene levels in carrots.

| | | Geronic | | Δ Geronic |
|---|---|---|---|---|
| Carrot | Time | Acid | β-carotene* | Acid (ng)/ |
| Physical State | (days) | (ng/g) | (μg/g) | Δ β-Carotene (μg) |
| Fresh | −1 | 4.9 ± 1.9 | n.d.** | — |
| Purée, dried | 0 | 97 ± 18 | 1366 | — |
| Powder, dried | 5 | 879 ± 56 | 1141 | 3.5 |
| Powder, dried | 12 | 3478 ± 252 | 697 | 5.9 |
| Powder, dried | 21 | 4155 ± 22 | 573 | 5.5 |

*The β-carotene measurement is approximate. The assay measures the absorbance of whole carrot extract at 454 nm, the maximum absorbance wavelength of β-carotene. However, there are smaller amounts of other compounds present, such as α-carotene and partially oxidized β- and α-carotenes that could contribute modestly to the absorbance at this wavelength.
**n.d.—not determined

### REFERENCES

(1) Ziegler, R. G. Vegetables, fruits, and carotenoids and the risk of cancer. Am. J. Clin. Nutr. 1991, 53, 251s-259s.
(2) Krinsky, N. I.; Johnson, E. J. Carotenoid actions and their relation to health and disease. Mol. Aspects Med 2005, 26, 459-516.
(3) Slavin, J. L.; Lloyd, B. Health benefits of fruits and vegetables. Adv. Nutr. 2012, 3, 506-516.
(4) Tang, G.; Russell, R. M. Carotenoids as provitamin A. In Carotenoids. Nutrition and Health, Britton, G., Liaaen-Jensen, S., Pfander, H., Eds.; Birkhäuser Verlag: Basel, 2009; Vol. 5, pp. 149-172.
(5) Peto, R.; Doll, R.; Buckley, J. D.; Sporn, M. B. Can dietary beta-carotene materially reduce human cancer rates? Nature 1981, 290, 201-208.
(6) Chew, B.; Park, J. The Immune System. In Carotenoids: Nutrition and Health, Britton, G., Liaaen-Jensen, S., Pfander, H., Eds.; Birkhäuser Verlag: Basel, 2009; Vol. 5, pp. 363-382.
(7) Bendich, A.; Olson, J. A. Biological actions of carotenoids. FASEB J. 1989, 3, 1927-32.
(8) Yeum, K.-Y.; Aldini, G.; Russell, R. M.; Krinsky, N. I. Antioxidant/pro-oxidant actions of carotenoids. In Carotenoids: Nutrition and Health, Britton, G., Liaaen-Jensen, S., Pfander, H., Eds.; Birkhäuser Verlag: Basel, 2009; Vol. 5, pp. 235-268.
(9) Young, A. J.; Phillip, D. M.; Lowe, G. M. Carotenoid antioxidant activity. In Carotenoids in Health and Disease, Krinsky, N. I., Mayne, S. T., Sies, H., Eds.; Marcel Dekker: New York, 2004; pp. 105-126.
(10) Britton, G. Functions of intact carotenoids. In Carotenoids: Natural Functions, Britton, G., Liaaen-Jensen, S., Pfander, H., Eds.; Birkhäuser Verlag: Basel, 2008; Vol. 4, pp. 189-212.
(11) Sayin, V. I.; Ibrahim, M. X.; Larsson, E.; Nilsson, J. A.; Lindahl, P.; Bergo, M. O. Antioxidants accelerate lung cancer progression in mice. Sci. Transl. Med 2014, 6, 221ra15.
(12) Harris, I. S.; Treloar, A. E.; Inoue, S.; Sasaki, M.; Gorrini, C.; Lee, K. C.; Yung, K. Y.; Brenner, D.; Knobbe-Thomsen, C. B.; Cox, M. A.; Elia, A.; Berger, T.; Cescon, D. W.; Adeoye, A.; Brustle, A.; Molyneux, S. D.; Mason, J. M.; Li, W. Y.; Yamamoto, K.; Wakeham, A.; Berman, H. K.; Khokha, R.; Done, S. J.; Kavanagh, T. J.; Lam, C. W.; Mak, T. W. Glutathione and thioredoxin antioxidant pathways synergize to drive cancer initiation and progression. Cancer Cell 2015, 27, 211-222.
(13) Burton, G. W.; Daroszewski, J.; Nickerson, J. G.; Johnston, J. B.; Mogg, T. J.; Nikiforov, G. B. β-Carotene autoxidation: oxygen copolymerization, non-vitamin A products and immunological activity. Can. J. Chem. 2014, 92, 305-316.
(14) Johnston, J. B.; Nickerson, J. G.; Daroszewski, J.; Mogg, T. J.; Burton, G. W. Biologically active polymers from spontaneous carotenoid oxidation. A new frontier in carotenoid activity. PLoS ONE 2014, 9, e111346.
(15) Miller, A. A.; Mayo, F. R. Oxidation of unsaturated compounds. I. The oxidation of styrene. J. Am. Chem. Soc. 1956, 78, 1017-1023.
(16) Duquette, S. C.; Fischer, C. D.; Feener, T. D.; Muench, G. P.; Morck, D. W.; Barreda, D. R.; Nickerson, J. G.; Buret, A. G. Anti-inflammatory benefits of retinoids and carotenoid derivatives: retinoic acid and fully oxidized 3-carotene induce caspase-3-dependent apoptosis and promote efferocytosis of bovine neutrophils. Am. J. Vet. Res. 2014, 75, 1064-1075.
(17) Boileau, A. C.; Erdman, J. W., Jr. Impact of food processing on content and bioavailability of carotenoids. In Carotenoids in Health and Disease, Krinsky, N. I., Mayne, S. T., Sies, H., Eds.; Marcel Dekker: New York, 2004; pp. 209-228.
(18) Britton, G.; Khachik, F. Carotenoids in food. In Carotenoids. Nutrition and Health, Britton, G., Liaaen-Jensen, S., Pfander, H., Eds.; Birkhäuser Verlag: Basel, 2009; Vol. 5, pp. 45-66.
(19) The NIST Mass Spectral Search Program for the NIST/EPA/NIH Mass Spectral Library Edition NIST 05, Version 2.0.
(20) Brooks, J.; Shaw, G. Chemical structure of the exine of pollen walls and a new function for carotenoids in nature. Nature 1968, 219, 532-533.
(21) Woodward, R. B. Structure and absorption spectra. IV. Further observations on α,β-unsaturated ketones. J. Am. Chem. Soc. 1942, 64, 76-77.
(22) Woodward, R. B. Structure and the absorption spectra of α,β-unsaturated ketones. J. Am. Chem. Soc. 1941, 63, 1123-1126.
(23) Lillya, C. P.; Kluge, A. F. Molecular spectra and conformations of conjugated dienones. J. Org. Chem. 1971, 36, 1977-1988.
(24) Baranska, M.; Schütze, W.; Schulz, H. Determination of lycopene and β-carotene content in tomato fruits and related products: Comparison of FT-Raman, ATR-IR, and NIR spectroscopy. Anal. Chem. 2006, 78, 8456-8461.
(25) Joseph, T. C.; Dev, S. Studies in sesquiterpenes-XXIX. Structure of himachalenes. Tetrahedron 1968, 24, 3809-3827.
(26) Bhan, P.; Pande, B. S.; Soman, R.; Damodaran, N. P.; Dev, S. Products active on arthropod-5. Insect juvenile hormone mimics: sesquiterpene acids having JH activity from the wood of cedrus deodara loud. Tetrahedron 1984, 40, 2961-2965.
(27) Hurnik, D.; Daroszewski, J.; Burton, G. W. In Determination of the effect of a fully oxidized β-carotene dietary supplement on the immune system and growth performance of weaned pigs, American Association of Swine Veterinarians 42nd Annual Meeting, Phoenix, Ariz., 2011; Phoenix, Ariz., 2011; pp. 277-279.
(28) Dietary Reference Intakes for Vitamin A, Vitamin K, Arsenic, Boron, Chromium, Copper, Iodine, Iron, Manganese, Molybdenum, Nickel, Silicon, Vanadium, and Zinc. Institute of Medicine. Food and Nutrition Board. National Academy Press: Washington, D.C., 2001.
(29) Takeoka, G. R.; Dao, L.; Flessa, S.; Gillespie, D. M.; Jewell, W. T.; Huebner, B.; Bertow, D.; Ebeler, S. E. Processing effects on lycopene content and antioxidant activity of tomatoes. J. Agric. Food Chem. 2001, 49, 3713-3717.
(30) Boileau, T. W.; Liao, Z.; Kim, S.; Lemeshow, S.; Erdman, J. W., Jr.; Clinton, S. K. Prostate carcinogenesis in N-methyl-N-nitrosourea (NMU)-testosterone-treated rats fed tomato powder, lycopene, or energy-restricted diets. J. Natl. Cancer Inst. 2003, 95, 1578-1586.
(31) Shaw, G. The chemistry of sporopollenin. In Sporopollenin, Brooks, J., Grant, P. R., Muir, M., Gijzel, P. V., Shaw, G., Eds.; Academic Press: London & New York, 1971; pp. 305-348.
(32) USDA National Nutrient Database for Standard Reference, Release 26. http://ndb.nal.usda.gov/. a) Entry 11655, Carrot juice, canned. b) Entry 11124, Carrots, raw. c) Entry 11683, Carrot, dehydrated. d) Entry 11529, Tomatoes, red, ripe, raw, year round average. e) Entry 09078, Cranberries, raw. f) Entry 35203, Rose hips, wild (Northern Plains Indians). g) Entry 11821, Peppers, sweet, red, raw. h) Entry 02028, Spices, paprika. i) Entry 11666, Seaweed, spirulina, raw. j) Entry 11667, Seaweed, spirulina, dried. k) Entry 11507, Sweet potato, raw, unprepared. l) Entry 01211, Milk, whole, 3.25% milkfat, without added vitamin A and vitamin D. m) Entry 01212, Milk, dry, whole, without added vitamin D. n) Entry 01123, Egg, whole, raw, fresh. o) Entry 01133, Egg, whole, dried.
(33) Canadian Nutrient File (Health Canada). http://webprod3.hc-sc.ac.ca/cnf-fce/index-ent.jsp. a) Food Code 2205—Tomato powder. b) Food Code: 2205—Seaweed, dulse (laver, nori), raw. c) Food code: 2499—Seaweed, dulse (laver, nori), dried.
(34) From manufacturer's product data sheet.
(35) Danish Food Composition Databank. http://www.foodcomp.dk/v7/fcdb_default.asp. FCDB no. 0095, Rosehip, dried, powder.
(36) Dillon, J. C.; Phan Phuc, A.; Dubacq., A. P. Nutritional Value of the Alga Spirulina. In World Review of Nutrition and Dietetics, Simopoulos, A. P., Ed.; Karger: Basel, Switzerland, 1995; Vol. 77, pp. 32-46.
(37) Al-Farsi, M. A.; Lee, C. Y. Nutritional and Functional Properties of Dates: A Review. Crit. Rev. Food Sci. 2008, 48, 877-887.
(38) Adapa, P.; Schoenau, G.; Tabil, L.; Sokhansanj, S.; Singh, A. Compression of fractionated sun-cured and dehydrated alfalfa chops into cubes: Pressure and density models. Can. Biosyst. Eng., 2005, 47, 3.33-3.39.
(39) Kandlakunta, B.; Rajendran, A.; Thingnganing, L. Carotene content of some common (cereals, pulses, vegetables, spices, and condiments) and unconventional sources of plant origin. Food Chem., 2008, 106, 85-89.
(40) Bauemfeind, J. C.; Adams, C. R.; Marusich, W. L. Carotenes and Other Vitamin A Precursors in Animal Feed. In Carotenoids as Colorants and Vitamin A Precursors, Technological and Nutritional Applications; Bauernfeind, J. C., Ed.; Academic Press: New York, 1981; pp. 563-743 (see Table 13, p. 623).
(41) Pardeshi, I. L.; Burbade, R. G.; Khod, R. N. Cost effective drying for high quality tender wheatgrass powder. J. Food Res. Technol. 2013, 1, 1-10. http://jakraya.com/journal/jfrt?pid=101.
(42) Ooi, C. K.; Choo, Y. M.; Yap, S. C.; Basiron, Y.; Ong, A. S. H. Recovery of carotenoids from palm oil. J. Am. Oil Chem. Soc. 1994, 71, 423-426. Also available at: https://hungermath.wordpress.com/2013/01/02/red-palm-oil-as-a-source-of-beta-carotene/.
(43) Taken from the manufacturer's nutritional information sheet for a similar product (Cranberry whole fruit powder, Milne Fruit Products, Prosser Wash. http://milnefruit.com/images/specsheets/nutritional/Milne_CranberryWFruitPowderNutr.pdf.
(44) Ciferri, Orio. Spirulina, the Edible Microorganism. Microbiological Reviews. December 1983, 47(4):551-578.
(45) Rajput, N. et al. Effect of Dietary Supplementation of Marigold Pigment on Immunity, Skin and Meat Color, and Growth Performance of Broiler Chickens. Brazilian Journal of Poultry Science. October-December 2012, 14(4):291-295.
(46) Koutsos, E. A. et al. Carotenoids from In Ovo or Dietary Sources Blunt Systemic Indices of the Inflammatory Response in Growing Chicks (Gallus gallus domesticus). The Journal of Nutrition. January 2006. pp. 1027-1031.
(47) Shanmugasundaram, R. et al. Lutein supplementation alters inflammatory cytokine production and antioxidant status in F-line turkeys. Poultry Science. 2011, 90:971-976.
(48) Pigmenter Specification Sheet for Oro Glo®.
(49) Saini, R. K. Carotenoid content in vegetative and reproductive parts of commercially grown Moringa oleifera Lam. Cultivars from India by LC-APCI-MS. European Food Research and Technology. June 2014. 238(6):971-978.
(50) Paguia, H. M. et al. Utilization and Evaluation of Moringa Oleifera L. As Poultry Feeds. Science Direct. 2014, pp. 343-347.
(51) Donkor, A. et al. Estimating the Nutritional Value of the Leaves of Moringaoleifera on Poultry. Food and Nutrition Sciences. 2013, 4:1077-1083.
(52) Abbas, T. E. The use of Moringa oleifera in poultry diets. Turkish Journal of Veterinary and Animal Sciences. 2013, 37:492-496.
(53) Burton, G. W.; Daroszewski, J; Mogg, T. J.; Nikoforov, G. B.: Nickerson, J. G. Discovery and Characterization of Carotenoid-Oxygen Copolymers in Fruits and Vegetables with Potential Health Benefits. J. Agric. Food Chem 2016 64, 3767-3777.
(54) G. Guiliano Plant carotenoids: genomics meets multi-gene engineering. Current Opinion in Plant Biology 2014, 19:111-117.

1. A method of identifying a source of carotenoid-oxygen copolymer comprising:
(a) selecting a food plant source or microorganism source containing carotenoids;
(b) processing the source under oxidative polymerization conditions; and
(c) quantifying the amount of carotenoid-oxygen copolymer by directly isolating or identifying same from said processed source and/or by isolating or identifying an indicator of same from said processed source, to determine whether it is a source of carotenoid-oxygen copolymer.

2. The method of claim 1, wherein the sources have a starting amount of carotenoid, which may provide upon oxidation the same amount of carotenoid-oxygen copolymer of 1-1000 μg/g wet weight or 10-10,000 μg/g dry weight.

3. The method of claim 1 or 2, wherein the oxidative polymerization conditions are selected from exposure to air or oxygen and one or more of drying, powdering, increasing exposure to heat, light, increasing the partial pressure of oxygen (ppO₂) or other factors that promote oxidation.

4. The method of any one of claims 1-3 wherein the isolation of carotenoid-oxygen copolymer comprises at least one polar organic solvent extraction/non-polar solvent precipitation cycles.

5. The method of claim 4 wherein the solvents are selected from solvents that are generally recognized as safe (GRAS).

6. The method of claim 4 or 5, wherein the polar organic solvent is ethyl acetate and the non-polar solvent is hexane.

7. The method of any one of claims 1 to 6, where the method of identifying is selected from one or more of: elemental analysis, GC-MS, GPC and FTIR.

8. The method of anyone of claims 1 to 7, wherein the food plant source is a plant or part thereof, a seed, a fruit or a vegetable.

9. The method of claim 8, wherein the food plant source is selected from the group consisting of: carrots, tomato, alfalfa, spirulina, rosehip, sweet pepper, chili pepper, paprika, sweet potato, kale, spinach, seaweed, wheatgrass, marigold, moringa oleifera and red palm oil.

10. The method of any one of claims 1 to 9, wherein the carotenoid has a β-ionone ring structure and the indicator is geronic acid.

11. The method of any one of claims 1 to 9, wherein the carotenoid is lutein, capsanthin or zeaxanthin and the indicator is 4-hydroxygeronic acid or its lactone.

12. The method of claim 11 wherein the carotenoid is lutein or zeaxanthin

13. The method of any one of claims 1 to 9 wherein the carotenoid is lycopene or γ-carotene and the indicator is geranic acid.

14. The method of any one of claims 1 to 9 wherein the carotenoid is canthaxanthin and the indicator is 2,2-dimethylglutaric acid or its anhydride thereof.

15. A method of enhancing the amount of carotenoid-oxygen copolymers available form a natural source selected from the group consisting of plants, algae, fungi, seeds, or microorganisms comprising:
(a) genetically modifying said natural source to enhance carotenoid production; and/or
(b) processing said natural source under oxidative polymerization conditions.

16. A method of preparing a product comprising carotenoid-oxygen copolymers comprising:
(a) obtaining a food plant source or microorganism source comprising carotenoids; and
(b) processing said source under oxidative polymerization conditions.

17. The method of claim 15 or 16, wherein the oxidative polymerization conditions are selected from exposure to air or oxygen and one or more of drying, powdering, increasing exposure to heat, light, increasing the partial pressure of oxygen (ppO₂), temperature and other factors that promote oxidation.

18. A method of isolating a carotenoid-oxygen copolymer product by subjecting the product obtained using the method of any one of claims 15 to 17 to one or more solvent extraction/precipitation cycles and recovering the carotenoid-oxygen copolymer containing fraction from same.

19. The method of claim 18, wherein in at least one polar organic solvent extraction/non-polar solvent precipitation cycle the solvents are selected from solvents that are generally recognized as safe (GRAS).

20. The method of claim 19, wherein the polar organic solvent is ethyl acetate and the non-polar solvent is a low molecular weight hydrocarbon.

21. The method of claim 20 wherein the low molecular weight hydrocarbon is hexane.

22. The method of any one of claim 16 to 21, wherein the food plant source is a plant or part thereof, a seed, a fruit or a vegetable.

23. The method of claim 22, wherein the food plant source is selected from the group consisting of: carrots, tomato, alfalfa, spirulina, rosehip, sweet pepper, chili pepper, paprika, sweet potato, kale, spinach, seaweed, wheatgrass, marigold, moringa oleifera and red palm oil.

24. A product prepared using the method of any one of claims 18 to 23.

25. The product of claim 24, wherein said product recovered after extraction/precipitation cycles does not comprise carotenoid breakdown products.

26. A composition comprising the product comprising carotenoid-oxygen polymers prepared in accordance with any one of claims 15 to 23 and suitable excipients.

27. A composition comprising the carotenoid-oxygen copolymer product isolated in accordance with any one claims 15 to 23 and suitable excipients.

28. An animal feed or supplement for an animal feed comprising the carotenoid-oxygen copolymer-comprising product prepared by the method of any one of claims 15 to 23.

29. A nutraceutical or supplement comprising carotenoid-oxygen copolymer-comprising product prepared by the method of any one of claims 15 to 23.

30. A method for enhancing carotenoid-oxygen copolymers in a carotenoid comprising food or supplement comprising the steps of adding to said food or supplement the carotenoid-oxygen copolymer product of claim 24 or 25.

31. Use of the carotenoid-oxygen copolymer product of claim 24 or 25 to enhance immunity in an animal.

32. Use of an effective amount of an isolated carotenoid-oxygen copolymer using the product of claim 24 or 25 to enhance animal health.

33. Use of claim 32, wherein the enhancement of animal health is selected from one or more of: enhancing innate immunity, enhancing anti-inflammation, enhancing the functioning of the immune system, enhancing the ability of an animal to resist disease, recover or overcome disease or maintain a healthy state.

34. The use of any one of claims 31 to 33, wherein the animal is a human.

35. A product that has a consistent, desired amount of carotenoid oxygen copolymer for the use of any one of claims 31 to 33, prepared using the method of any one of claims 15 to 23.

36. A naturally sourced OxPVA composition free from norisoprenoid by-products.

37. A naturally sourced OxCar composition free from norisoprenoid by-products.

38. A composition of claim 36 or 37 derived from processing a carotenoid comprising natural source under oxidative polymerization conditions and subjecting same to one or more solvent extraction/precipitation cycles and recovering the carotenoid-oxygen copolymer containing fraction from same.

39. The composition of claim 38 wherein the natural source is genetically modified to enhance carotenoid production.
